SEQUENCE LISTING (5 sequences)

SEQ ID NO: 1 - protein, 474 aa, synthetic construct
  source 1..474: note = MARCO ECD
MGWSCIILFLVATATGVHSHHHHHHQVLNLQEQLQMLEMCCGNGSLAIEDKPFFSLQWAPKTHLVPRAQGLQALQAQLSWVHTSQEQLRQQFNNLTQNPELFQIKGERGSPGPKGAPGAPGIPGLPGPAAEKGEKGAAGRDGTPGVQGPQGPPGSKGEAGLQGLTGAPGKQGATGAPGPRGEKGSKGDIGLTGPKGEHGTKGDKGDLGLPGNKGDMGMKGDTGPMGSPGAQGGKGDAGKPGLPGLAGSPGVKGDQGKPGVQGVPGPQGAPGLSGAKGEPGRTGLPGPAGPPGIAGNPGIAGVKGSKGDTGIQGQKGTKGESGVPGLVGRKGDTGSPGLAGPKGEPGRVGQKGDPGMKGSSGQQGQKGEKGQKGESFQRVRIMGGTNRGRAEVYYNNEWGTICDDDWDNNDATVFCRMLGYSRGRALSSYGGGSGNIWLDNVNCRGTENSLWDCSKNSWGNHNCVHNEDAGVECS

SEQ ID NO: 2 - protein, 123 aa, synthetic construct
  source 1..123: note = MARCO SRCR
MGWSCIILFLVATATGVHSHHHHHHQRVRIMGGTNRGRAEVYYNNEWGTICDDDWDNNDATVFCRMLGYSRGRALSSYGGGSGNIWLDNVNCRGTENSLWDCSKNSWGNHNCVHNEDAGVECS

SEQ ID NO: 3 - protein, 376 aa, synthetic construct
  source 1..376: note = MARCO ECD without the SRCR
MGWSCIILFLVATATGVHSHHHHHHQVLNLQEQLQMLEMCCGNGSLAIEDKPFFSLQWAPKTHLVPRAQGLQALQAQLSWVHTSQEQLRQQFNNLTQNPELFQIKGERGSPGPKGAPGAPGIPGLPGPAAEKGEKGAAGRDGTPGVQGPQGPPGSKGEAGLQGLTGAPGKQGATGAPGPRGEKGSKGDIGLTGPKGEHGTKGDKGDLGLPGNKGDMGMKGDTGPMGSPGAQGGKGDAGKPGLPGLAGSPGVKGDQGKPGVQGVPGPQGAPGLSGAKGEPGRTGLPGPAGPPGIAGNPGIAGVKGSKGDTGIQGQKGTKGESGVPGLVGRKGDTGSPGLAGPKGEPGRVGQKGDPGMKGSSGQQGQKGEKGQKGESF

SEQ ID NO: 4 - protein, 123 aa, synthetic construct
  source 1..123: note = SRCR domain with three arginine mutations
MGWSCIILFLVATATGVHSHHHHHHQRVAIMGGTNAGAAEVYYNNEWGTICDDDWDNNDATVFCRMLGYSRGRALSSYGGGSGNIWLDNVNCRGTENSLWDCSKNSWGNHNCVHNEDAGVECS

SEQ ID NO: 5 - protein, 105 aa, synthetic construct
  source 1..105: note = SRCR domain with the arginine-rich region deleted
MGWSCIILFLVATATGVHSHHHHHHNNEWGTICDDDWDNNDATVFCRMLGYSRGRALSSYGGGSGNIWLDNVNCRGTENSLWDCSKNSWGNHNCVHNEDAGVECS

(19) United States
(12) Patent Application Publication
(10) Pub. No.: US 2026/0102468 A1
(43) Pub. Date: Apr. 16, 2026

(54) COMPOSITIONS AND METHODS FOR MARCO INHIBITION AND IMPROVED DRUG DELIVERY

(71) Applicant: BOARD OF REGENTS, THE UNIVERSITY OF TEXAS SYSTEM, Austin, TX (US)

(72) Inventors: Wen JIANG, Houston, TX (US); Yifan WANG, Houston, TX (US); Yon Son Betty KIM, Houston, TX (US)

(21) Appl. No.: 19/116,200

(22) PCT Filed: Sep. 28, 2023
(86) PCT No.: PCT/US2023/075419

§ 371 Date: Mar. 27, 2025

Related U.S. Application Data
(60) Provisional application No. 63/377,422, filed on Sep. 28, 2022.
(60) Provisional application No. 63/580,783, filed on Sep. 6, 2023.

(51) Int. Cl. A61K 38/17; A61K 9/00; A61K 9/127; A61K 9/51; A61K 31/337; A61K 31/4745; A61K 31/704; A61K 31/7105; A61K 31/711; A61P 35/04; C07K 16/00; G01N 33/68

(52) U.S. Cl. CPC A61K 38/177; A61K 9/0019; A61K 9/127; A61K 9/51; A61K 31/337; A61K 31/4745; A61K 31/704; A61K 31/7105; A61K 31/711; A61P 35/04; C07K 16/00; G01N 33/6893

(57) ABSTRACT

According to various aspects of this disclosure, the present disclosure relates to combination therapies, methods, and kits comprising MARCO blocking agents and anti-cancer agents for improvement of nanoformulated drug delivery. Further, wherein a method of treating a tumor or cancer in a subject in need thereof comprising administering to the subject a therapeutically effective amount of a combination therapy comprising a first agent and a second agent, wherein the first agent is capable of blocking the inter-action between a Macrophage Receptor with Collagenous Structure (MARCO) and a nanoparticle, and the second agent comprises an anti-cancer agent or an anti-tumor agent, and wherein at least the second agent is in the form of a nanoformulation, is disclosed.

### CROSS-REFERENCE TO RELATED APPLICATIONS

This International Application claims the priority benefit of U.S. Provisional Application No. 63/377,422, filed on Sep. 28, 2022, and U.S. Provisional Application No. 63/580,783, filed on Sep. 6, 2023, each of which is incorporated herein by reference in its entirety.

### REFERENCE TO A SEQUENCE LISTING SUBMITTED ELECTRONICALLY VIA EFS-WEB

This application includes a Sequence Listing submitted electronically via EFS-Web (name: “4443_023PC02_Seqlisting_ST26.xml”; size: 7,569 bytes; and created on: Sep. 7, 2023), which is hereby incorporated by reference in its entirety.

### FIELD OF THE DISCLOSURE

The present disclosure relates to the field of drug delivery and oncology. Certain aspects of the disclosure are directed to inhibition of MARCO activity for the improvement of nanoformulated drug delivery.

### BACKGROUND

Cancer remains a leading cause of death, and current therapies for many cancers are inadequate. Chemotherapy is a conventional and widely used cancer treatment method. While chemotherapy works through a number of different mechanisms, its major function includes indiscriminately killing quickly growing cells, including tumor and normal cells, which causes side effects. Other cancer therapies such as antibodies and nucleic acid therapies have proven to be effective or are in development for certain cancers.

Nanotechnology has been increasingly used in medicine, including applications for diagnosis, treatment, and tumor targeting in a safer and more effective manner (Yao et al., 2020, Mol. Biosci.). Nanoparticle (NP)-based drug delivery systems have shown many advantages in cancer treatment, such as good pharmacokinetics, precise targeting of tumor cells, reduction of side effects, and drug resistance (Dadwal et al., 2018, Artif. Cells Nanomed. Biotechnol. 46, 295-305); Palazzolo et al., 2018, Curr. Med. Chem. 25, 4224-4268). Nanoparticles used in drug delivery systems are typically designed or chosen based on their size and characteristics according to the pathophysiology of the tumors.

Mechanically, nano-carriers in cancer therapy target to tumor cells through the carrier effect of the nanoparticles and the positioning effect of the targeting substance after being absorbed. Next, they release the drugs to tumor cells in order to induce killing. Drugs located on the inside of the nano-carriers include traditional chemotherapy agents and nucleic acids, indicating that they can play a role in both cytotoxic and gene therapy (Chen et al., 2015, Adv. Drug Deliv. Rev. 81, 128-141). In addition, for some poorly soluble drugs, NPs offer a platform that can help encapsulate them and deliver the drugs into circulation (Kipp, 2004, Int. J. Pharm. 284, 109-122; Zhang et al., 2008, ACS Nano 2, 1696-1702). Due to the size and surface characteristics of nanoparticles and their function of enhancing permeability and retention, nano-carriers can increase the half-life of drugs and induce their accumulation into tumor tissues (Bertrand et al., 2014, Adv. Drug Deliv. Rev. 66, 2-25; Kalyane et al., 2019, Mater. Sci. Eng. C Mater. Biol. Appl. 98, 1252-1276).

Macrophage receptor with collagenous structure (MARCO) is a class A scavenger receptor molecule expressed and localized at the cell membrane in macrophages (Kraal, G. et al. 2000. Microbes and Infection. 2 (3): 313-316). MARCO efficiently binds and facilitates cellular import and degradation of foreign material such as bacteria. MARCO blockade has also been shown to significantly decreased nanoparticle internalization (Park et al., 2019, PNAS 116 (30): 14947-14954). MARCO expression can be stimulated by infection or the presence of foreign objects (Grolleau, A., et al. 2003. J Immunol 171, 2879-2888). It has been reported that MARCO is the major receptor on alveolar macrophages for unopsonized particles (Arredouani, M. S. et al. 2005. J Immunol 175, 6058-6064) and its expression is lower on alveolar macrophages collected from aged mice than young (Li, Z. et al. 2017. J Immunol 199, 3176-3186), but whether the MARCO expression on liver macrophages changes with aging and its impact on age-dependent nanoparticle clearance is unknown.

### DESCRIPTION OF FIGURES

FIGS. 1A-1C: Show the correlation of MARCO expression with macrophages' ability to uptake nanoparticles. FIG. 1A: shows immunofluorescence staining of bone marrow-derived macrophages (BMDMs) incubated with nanoparticles. Scale bar=5 μm. FIG. 1B: shows nanoparticle uptake by the MARCO-positive and MARCO-negative BMDMs; Left panel shows representative flow cytometry results. n=3. Unpaired t-test. FIG. 1C: Shows flow cytometry results showing MARCO expression on macrophages; Right panel: phagocytosis of nanoparticles.

FIGS. 2A-2B: Show that knockdown of MARCO decreases nanoparticle uptake by macrophages. FIG. 2A: shows flow cytometry results of MARCO knockdown by siRNA. FIG. 2B: shows nanoparticle phagocytosis by macrophages after knockdown.

FIGS. 3A-3C: Show blockade of MARCO-nanoparticle interaction decreases nanoparticle uptake by macrophages. FIG. 3A: shows images of macrophages and nanoparticles incubated with or without the anti-MARCO antibody (5 μg/ml) or isotype IgG. Scale bar=50 μm. White arrows indicate cells that have taken up nanoparticles. FIG. 3B: shows quantification of nanoparticle⁺ cells. Unpaired t-test. All error bars are mean±standard deviation (S.D.) FIG. 3C: shows nanoparticle uptake by macrophages with or without incubation with recombinant MARCO protein.

FIGS. 4A-4B: Show blockade of MARCO-nanoparticle interaction decreases nanoparticle uptake by liver. FIG. 4A: shows in vivo imaging (IVIS images collected with a PERKINELMER® IVIS® imaging system, which can detect and quantify the fluorescence emitted by the nanoparticle) of liver at 4 hours after nanoparticle administration. FIG. 4B: shows normalized total radiance (emission) of liver. Unpaired t-test. Error bars are mean±S.D.

FIGS. 5A-5C: Show blockade of MARCO increased the therapeutic effect of ABRAXANE® in a breast cancer mouse model study. FIG. 5A: shows growth curves and FIG. 5B: shows final tumor volume of E0771 tumor cells implanted in female C57BL6 mice treated by paclitaxel-protein particles (ABRAXANE®) or ABRAXANE® plus MARCO blockade. n=5-6 mice in each treatment arm. Un-paired t-test was used to compare tumor volumes at day 18 after ABRAXANE® vs. ABRAXANE®+MARCO blockade. Error bars are mean±S.E.M. AB, anti-MARCO antibody; ECD (extracellular domain), MARCO protein extracellular domain; SRCR, MARCO protein scavenger receptor cysteine-rich (SRCR) domain. FIG. 5C: shows that treatment efficacy of ABRAXANE® is not enhanced by MARCO blockade in older (50 weeks old) C57BL6 mice, which have lower MARCO liver and blood expression.

FIGS. 6A-6B: Show blockade of MARCO increased the therapeutic effect of ABRAXANE® in a lung cancer mouse model study. FIG. 6A: shows growth curves of LKR13-LKB1-KO lung tumor cells implanted in male 129S1 mice treated by paclitaxel-protein particles (ABRAXANE®) or ABRAXANE® plus MARCO blockade. n=5-6 mice in each treatment arm. Un-paired t-test was used to compare tumor volumes at day 8. Error bars are mean±S.E.M. FIG. 6B: shows survival curves of mice in each treatment groups. Log-rank test was used to compare the survival probability between ABRAXANE® vs. ABRAXANE®+MARCO blockade.

FIG. 7: Shows blockade of MARCO increased the tumor delivery of ABRAXANE®. Comparisons of body weight, tumor size, and relative tumor concentration of paclitaxel (determined by mass spectrometry) among mice injected with ABRAXANE® or ABRAXANE®+MARCO-SRCR. Un-paired t-test.

FIGS. 8A-8G: Show blockade of MARCO decreased the toxicity of ABRAXANE®. FIGS. 8A-8B: show body weight change of 129S1 mice treated by ABRAXANE® or ABRAXANE®+MARCO recombinant protein. Unpaired t-test. FIGS. 8C-8G: show blood liver and kidney toxicity marker levels in different groups of treated mice compared to control. The toxicity markers included aspartate aminotransferases (AST) (FIG. 8C), alanine aminotransferases (ALT) (FIG. 8D), alkaline phosphatase (ALP) (FIG. 8E), creatinine (CREA) (FIG. 8F), and blood urea nitrogen (BUN) (FIG. 8G). One-way ANOVA with post hoc corrections for multiple comparisons.

FIGS. 9A-9D: Show blockade of MARCO increased the therapeutic effect of DOXIL® in a breast cancer mouse model study. FIG. 9A: shows growth curves of E0771 breast tumor cells implanted in C57BL6 mice treated by DOXIL® or DOXIL® plus MARCO blockade. n=5-6 mice in each treatment arm. Un-paired t-test was used to compare tumor volumes at day 18. Error bars are mean±S.E.M. FIG. 9B: shows body weight change. FIG. 9C: shows body weight at week 3. FIG. 9D: shows that treatment efficacy of DOXIL® is not enhanced by MARCO blockade in older (50 weeks old) C57BL6 mice, which have lower MARCO liver and blood expression.

FIGS. 10A-10H: Show differences in drug and nanoparticle responses between young and old mice. Growth curves of E0771 breast tumor cells implanted in young or old C57BL6 mice treated with saline (control) (FIG. 10A), nanoparticle albumin-bound paclitaxel (nab-paclitaxel) (FIG. 10B), or liposomal doxorubicin (FIG. 10C). Tumor volumes at day 18 were compared. FIG. 10D shows quantification of relative tumor/liver doxorubicin concentrations. The ratio of relative concentration was calculated by total emission normalized by organ weight. n=4 mice in each group. FIG. 10E shows quantification of doxorubicin⁺ percentages in the CD11b+ and CD11b-liver cell populations. n=8 mice. FIG. 10F shows the percentage of liver CD11b⁺ cells from young or old mice with positive doxorubicin signal detected by flow cytometry. n=4 mice in each age group. FIG. 10G shows a summarized result of the percentage of CD11b and CD11b-liver cells with liposome uptake. n=6 mice. FIG. 10H shows the results of a comparison of phagocytosis of nanoparticles by old and young liver CD11b cells. n=3 mice in each group. Two-sided unpaired/tests were used. Error bars are means±S.E.M. in FIGS. 10A-10C. Error bars are means±S.D. in FIGS. 10D-10H.

FIGS. 11A-11B: show an analysis of tumor blood vessels in young versus old mice. FIG. 11A shows representative staining of young and old tumor slides. Scale bars=50 μm. FIG. 11B shows the quantification of fluorescence intensity of endothelial marker (CD31) and pericyte marker (NG2) of E0771 tumor collected from young or old C57BL6 mice. Tumors were collected from n=3 mice each group. 4 random fields of each tumor were analyzed. Two-sided unpaired/test. Error bars are showing means±SD.

FIGS. 12A-12B: show flow cytometry results of tumor associated macrophages (TAM) in young versus old mice. FIG. 12A shows the percentage of CD11b⁺;F4/80⁺TAM among all cells in the E0771 tumor implanted into young or old female C57BL6 mice. n=5 mice in each group. Two-sided unpaired/test. Error bars are showing means±SD. FIG. 12B shows the gating strategy of flow cytometry analysis of TAM.

FIGS. 13A-13B: show flow cytometry analysis of phagocytosis in young versus old TAM. FIG. 13A shows the percentage of CD11b⁺;F4/80⁺ TAM with nanoparticle uptake after 2 h incubation. n=5 mice each group. Two-sided unpaired/test. Error bars are showing means±SD. FIG. 13B shows the gating strategy of flow cytometry analysis of TAM phagocytosis.

FIGS. 14A-14D: show nanoparticle characterization results. FIGS. 14A-14B show particle sizes of liposomal doxorubicin and empty liposomes as determined by Nanosight. FIGS. 14C-14D show MTS assay results of the cytotoxicity of liposomal doxorubicin and empty liposome in the mouse macrophage cell lines J774A. 1 and RAW264.7. The concentration refers to the drug concentration of doxorubicin. The empty liposome (eLNP) was diluted to the same nanoparticle concentration as liposomal doxorubicin. n=3 biological independent replicates. Error bars are means±S.D. Two-sided unpaired/tests.

FIGS. 15A-15B: show results of drug uptake comparisons in CD11b⁺ cells from mouse livers. FIG. 15A shows fluorescence emission of FITC-labeled nab-paclitaxel. n=3 independent biological repeats. Error bars are means±SD. FIG. 15B shows phagocytosis of FITC-labeled nab-paclitaxel by liver CD11b⁺ cells. All experiments were independently repeated biologically with n=4 pairs of young and old animals. Error bars are means±S.D. Differences between groups were determined by two-sided unpaired/tests.

FIGS. 16A-16B: show measured particle sizes of 50-nm nanoparticles. Nanoparticle sizes were measured by Nanosight before (FIG. 16A) and after (FIG. 16B) PEGylation. Five technical replications were performed.

FIGS. 17A-17B: show measured particle sizes of 100-nm nanoparticles. Nanoparticle sizes were measured by Nanosight before (FIG. 17A) and after (FIG. 17B) PEGylation. Five technical replications were performed.

FIGS. 18A-18C: show single-cell RNAseq (scRNAseq) workflow overview and metrics. FIG. 18A shows a schematic of the scRNA-seq workflow, generated with elements downloaded from Servier Medical Art, provided by Servier, under a Creative Commons Attribution 3.0 unported license. PBS or 10¹¹ nanoparticles/20 g of body weight were injected intravenously in young or old C57BL6 mice. FIG. 18B shows information for samples used in the scRNAseq study. FIG. 18C shows violin plots indicating the number of detected unique molecular identifiers (UMIs) (left) and genes (right) in an individual cell. The lower and upper hinges correspond to the first and third quartiles, and the center refers to the median value. The upper/lower whiskers extend from the hinge to the largest/smallest value. The n numbers of single cells in each cluster are noted underneath each cluster in the parentheses. QC, quality control. Numbers of independent mice: YC, young-control, n=3; YN, young-nanoparticle, n=4; OC, old-control, n=2; ON, old-nanoparticle, n=3.

FIGS. 19A-19F: show scRNAseq results in liver macrophages from young and old mice. FIG. 19A shows a Uniform Manifold Approximation and Projection (UMAP) visualization of the six macrophages clusters in livers from mice in the control groups. KC: Kupffer cells; MDM: Monocytes-derived macrophages; LAM: Lipid associated macrophages; NCM: Non-classical monocytes; rM: Resolution-phase macrophages; mM: Mitotic macrophages. FIG. 19B shows the sample origin of the liver macrophages. FIG. 19C shows violin plots of signature genes expressed in each cluster of liver macrophages. The lower and upper hinges correspond to the first and third quartiles, and the center refers to the median value. The upper/lower whiskers extend from the hinge to the largest/smallest value. FIG. 19D shows UMAP embedding visualization of macrophages from mouse liver tissues across control (C) and nanoparticle (N)-treated groups. Color indicates clustering. FIG. 19E shows expression of phagocytosis-related genes in all treatment groups. The average expression level is shown by color scale; the percentage of positive cells is shown by dot size. FIG. 19F shows the visualized expression patterns of Marco on liver macrophages in the control- and nanoparticle-treated groups. The color scale indicates expression intensity. YC, young-control, n=3 mice; YN, young-nanoparticle, n=4 mice; OC, old-control, n=2 mice; ON, old-nanoparticle, n=3 mice.

FIG. 20: shows a dot plot depicting the relative average expression levels of representative genes identified in the cell populations noted on the y-axis. Color intensity represents the average level of expression: red, high expression; grey, low expression. Size of dot represents the percent of gene expression.

FIG. 21: shows a dot plot depicting the relative average expression levels of representative genes identified in indicated macrophage populations. Color intensity represents the average level of expression: red, high expression; grey, low expression. Size of dot represents the percent of gene expression.

FIGS. 22A-22F: show expression patterns of genes related to antigen presenting cells' function in liver macrophages. Genes were grouped as associated with lysosome (FIG. 22A), antigen presentation (FIG. 22B), cell adhesion (FIG. 22C), lipid and cholesterol metabolism (FIG. 22D), complement (FIG. 22E), and chemokine/cytokine (FIG. 22F). Color intensity represents the relative average level of expression: red, high expression; grey, low expression. Size of dot represents the percent of gene expression. YC, young-control; YN, young-nanoparticle; OC, old-control; ON, old-nanoparticle.

FIGS. 23A-23D: show Marco expression in each cluster of liver macrophages. FIG. 23A shows Marco expression in the YC, YN, OC, and ON groups. FIG. 23B shows Marco expression in each treatment group. FIG. 23C shows the percentages of Marco⁺ macrophages (among all liver macrophages) in the four treatment groups. FIG. 23D shows Marco expression level in each individual cell in cluster 1 and 6. Two-sided unpaired/test was used to compare the differences between groups. YC, young-control; YN, young-nanoparticle; OC, old-control; ON, old-nanoparticle. The lower and upper ends of each column represent the minimum and maximum values of the data. The lower and upper ends of the middle box plot represent the first and third quartiles, and the horizontal line inside the box represents the median. The n numbers of single cells in each cluster or group are noted underneath each cluster or group in the parentheses. Numbers of independent mice: YC, n=3; YN, n=4; OC, n=2; ON, n=3.

FIG. 24: shows a UMAP indicating the expression of Mki67 in each of the indicated groups. YC, young-control; YN, young-nanoparticle; OC, old-control; ON, old-nanoparticle. Color intensity represents the average level of expression: red, high expression; grey, low expression.

FIGS. 25A-25J: shows expression of MARCO on liver macrophages relative to age. FIG. 25A shows the expression of MARCO on mouse CD11b⁺ cells from liver and peripheral blood mononuclear cells (PBMCs) from young (8-week-old) and old (50-week-old) mice. FIG. 25B shows the quantification of flow cytometry results on MARCO expression in liver and PBMCs. n=3 mice in each group. FIG. 25C shows representative immunofluorescence staining of livers from young and old mice. n=5 biologically independent mice in each group, representative staining result is shown. Red, MARCO; Blue, 4′,6-diamidino-2-phenylindole (DAPI) nuclear stain. Scale bar=20 μm. FIG. 25D shows quantification of MARCO immunofluorescence staining intensity in young and old mouse livers. n=5 mice in each age group. FIG. 25E shows the percentage of liver macrophages expressing MARCO with or without nanoparticle injection in young and old C57BL6 mice. Numbers of biologically independent mice: Young-PBS, n=5; Young-NP, n=7; Old-PBS, n=6; Old-NP, n=7. FIG. 25F shows immunofluorescence staining results of livers from young and old monkeys (Macaca fascicularis). n=3 biologically independent monkeys from each age group, representative staining result is shown. Scale bar=20 μm. Green, CD11b; Red, MARCO; Blue, DAPI. FIG. 25G shows quantification of immunofluorescence staining results. n=3 monkeys from each age group, 3 random fields were analyzed for each sample. FIG. 25H shows MARCO transcription levels of whole liver samples from young (<50-year-old, n=45 biologically independent samples) and older (>50-year-old, n=130 biologically independent samples) patients. pTPM, protein transcripts per million. FIG. 25I shows expression of MARCO on peripheral blood mononuclear cells (PBMCs) from young (<40 years) or older (>40 years) human donors. n=4 donors in each group. FIG. 25J shows MARCO expression on human livers of different ages. Data is from 7 biologically independent individuals, with 3 randomly selected fields for each. Simple linear regression was used to test the association between age and expression. The P values reflect whether the slope is significantly non-zero. Two-sided unpaired/tests were used in FIGS. 25B, 25D-25E, and 25G-25I. All error bars are means±SD.

FIGS. 26A-26G: show MARCO expression is associated with nanoparticle uptake by macrophages. FIG. 26A shows a heatmap of RNAseq results showing the genes expressed at the most different levels between bone marrow-derived macrophages (BMDMs) from young and old mice. n=6 mice from each age group. FIG. 26B shows RT-PCR validation of Marco gene transcription. n=6 mice from each age group. FIG. 26C shows the relative Marco mRNA levels in young and old BMDMs after a 4-hour incubation with nanoparticles. n=3 biological replications. FIG. 26D shows a representative image of young and old BMDMs stained by MARCO antibodies. Dashed boxes indicate the amplified areas shown at right. Scale bar=50 μm (left panel), 10 μm (right panel). Experiment was repeated independently for 3 times with similar results. Representative result is shown. FIG. 26E shows quantification of MARCO foci in MARCO⁺ macrophages. Twelve randomly chosen areas from each group were analyzed. Young, n=37 cells; Old, n=27 cells examined over 3 independent experiments. FIG. 26F shows a Western blot of albumin from the pull-down by MARCO. Experiment was repeated independently for 3 times with similar results. Representative result is shown. FIG. 26G shows a Western blot of albumin indicating the amount of unbound nab-paclitaxel in the supernatant after incubation with saturated dose of recombinant MARCO. Experiment was repeated independently for 3 times with similar results. Representative result is shown. Two-sided unpaired/tests were used in FIGS. 26B-26C, and 26E. All error bars are means±S.D.

FIGS. 27A-27C: show uptake of nanoparticles by bone marrow-derived macrophages (BMDMs) from young and old mice. FIG. 27A shows the uptake of 50-nm and 100-nm PEGylated nanoparticles by BMDMs from young and old mice. Experiments were independently repeated with 3 pairs of young and old animals. Error bars are means±S.D. Differences between groups were determined by two-sided unpaired t tests. FIG. 27B shows the uptake of liposomal doxorubicin (Doxil) by BMDMs from young and old mice. n=3 mice each group. Error bars are means±SD. Two-sided unpaired t test. FIG. 27C shows the quantification of flow cytometry assay of empty liposome uptake by BMDM cells treated with anti-MARCO antibody or IgG. n=3 biological replicates. Two-sided unpaired t test. Error bars are means±SD.

FIGS. 28A-28B: show MARCO protein binding to nanoparticles results, wherein the experimental strategy includes using a 30 kDa cutoff filter to separate unbound MARCO (SRCR) protein from nanoparticle-bound protein via centrifugation. FIGS. 28A-28B show Western blot analysis of the unbound MARCO at the bottom of the centrifugal filter tube after the recombinant MARCO (0.1 mg/ml SRCR) was incubated with different concentrations of nab-paclitaxel (FIG. 28A) or 100 nm nanoparticles (FIG. 28B) followed by centrifugation. Experiment was repeated independently for 3 times with similar results. Representative result is shown.

FIG. 29: shows tumor growth curves of E0771 tumor cells implanted in old mice treated with either saline+IgG, Anti-MARCO antibody, nab-paclitaxel (ABP)+IgG, or ABP+Anti-MARCO antibody. n=5 mice per group. One-way analysis of variance with post hoc testing was used to compare tumor volumes at day 18 after treatment.

FIG. 30: shows MARCO blockade increases nanoparticle blood circulation time. Anti-MARCO, monoclonal MARCO antibody, clone ED31, 2.5 mg/kg; rMARCO, recombinant MARCO protein SRCR domain, 0.3 mg/kg; NP, 100 nm nanoparticles, 10¹¹ particles injected through tail vain. n=3 mice for each treatment group at each time point. One way ANOVA with correction for multiple comparison was used to compare the difference between NP and other treatment groups. Error bars are means±SD.

FIGS. 31A-31D: shows role of SRCR domain of MARCO in improving nanomedicine efficacy. FIG. 31A shows baseline tumor volumes of all groups before treatment. Error bars are means±SD. One-way ANOVA was used to analyze the differences of tumor volume at baseline. n=6 biologically independent mice in each group. FIG. 31B shows summarized tumor groups curves with indicated treatment. n=6 biologically independent mice in each group. Error bars are means±SEM. One-way ANOVA was used to analyze the differences of tumor volume at day 18. FIG. 31C shows baseline tumor volume of all groups before treatment. Error bars are means±SD. Two-sided unpaired/test was used to analyze the differences of tumor volume at baseline. n=5 biologically independent mice in each group. FIG. 31D shows summarized tumor groups curves with indicated treatment. n=5 biologically independent mice in each group. Error bars are means±SEM. Two-sided unpaired/test was used to analyze the differences of tumor volume at day 18. ABP, nab-paclitaxel, 20 mg/kg; SRCR, recombinant SRCR domain of MARCO, 0.3 mg/kg or 3 mg/kg); mSP, mouse serum protein, 1.2 mg/kg; ECD^{Δ}, truncated recombinant MARCO ECD domain without the SRCR domain, 1.2 mg/kg.

FIGS. 32A-32B: show ABRAXANE®+MARCO recombinant protein inhibited metastasis of LKR13-LKB1-KO cells. FIG. 32A shows the numbers of lung metastases, counted on lung tissue slides. P values are from one-way analysis with post hoc corrections. The n number of biologically independent mice in each group is noted in the figure. Error bars are means±SD. FIG. 32B shows H&E staining images of representative lung slides in each group. The displayed area is representative of the whole lung. Black arrows indicate metastatic tumors. Scale bar=2 mm. ABP, nab-paclitaxel; MARCO, recombinant MARCO SRCR domain; ABP-M, ABP+recombinant MARCO SRCR domain.

FIGS. 33A-33F: show effect of MARCO blockade on Doxil response. FIG. 33A shows tumor growth curves of E0771 tumors implanted in young mice treated as indicate with either IgG+saline, anti-MARCO antibody, Dox+IgG, or Dox+anti-MARCO antibody. n=6 mice per group. FIG. 33B shows tumor growth curves of E0771 tumors implanted in young mice treated as indicate with either Control, MARCO, Dox, or Dox+MARCO. n=6 mice per group. One-way analysis of variance with post hoc testing was used to compare tumor volumes at day 18 after treatment. MARCO: recombinant MARCO SRCR domain; Dox: liposomal doxorubicin; Dox-M, liposomal doxorubicin plus MARCO-SRCR blockade. FIGS. 33C-33D show survival curves of young and old mice bearing E0771 tumors in the indicated treatment groups. P values for the Dox vs. Dox-M group were determined by log-rank tests. Day zero is the day of tumor implantation. Dox-M, liposomal doxorubicin plus MARCO-SRCR blockade. Numbers of biologically independent mice in each group: Young: Control and Dox-M, n=6 each; MARCO and Dox, n=5 each; Old: Control and MARCO, n=6 each; Dox and Dox-M, n=7 each. FIGS. 33E-33F show quantification of the relative Doxil fluorescence intensity per mg of tissue, and IVIS images of liver and tumor in mice treated with liposomal doxorubicin (Dox) or Dox plus MARCO-SRCR blockade (Dox-M). n=3 mice each group. P value is from two-sided unpaired/test. Error bars are means±SD.

FIGS. 34A-34B: show recombinant MARCO administration with Doxil in old mice. FIG. 34A shows weekly body weight changes in old C57BL6 mice treated with liposomal doxorubicine (Dox) or Dox plus MARCO-SRCR blockade (Dox-M). FIG. 34B shows body weight changes at week 3. P values are from one-way analysis of variance with Tukey correction for multiple tests. Each dot represents one mouse. Error bars are means±SD. Numbers of biologically independent mice in each group are noted in the figure.

FIGS. 35A-35E: show blockage of MARCO increases the maximum tolerated dose of Doxil in young mice. FIGS. 35A-35B show severe toxicity-free survival of young mice bearing E0771 tumors treated by liposomal doxorubicin with (FIG. 35B) or without (FIG. 35A) anti-MARCO antibody (2.5 mg/kg). Indicated dose (4 mg/kg, 6 mg/kg, or 8 mg/kg) of liposomal doxorubicin or saline was injected every three days for total of 5 injections. n=5 mice each group. P values are from log rank test. FIG. 35C shows severe toxicity-free survival of mice treated with indicated dose (4 mg/kg or 6 mg/kg) of free doxorubicin, with or without anti-MARCO antibody. n=5 mice each group. P values are from log rank test. FIG. 35D shows severe toxicity-free survival of old mice bearing E0771 tumors treated by liposomal doxorubicin with or without anti-MARCO antibody (2.5 mg/kg). n=5 mice each group. P values are from log rank test. All survival curves were nudged to make overlapped lines visible. FIG. 35E shows growth curves of E0771 tumors implanted in young mice. n=5 mice each group. Error bars are means±SEM. P values are from one-way analysis of variance with correction for multiple tests.

### BRIEF SUMMARY OF THE INVENTION

Certain aspects of the disclosure are directed to compositions and methods comprising co-administration of MARCO blocking molecules and nanoformulated therapeutic agents for improved uptake of the nanoformulated therapeutic agents in order to improve efficacy and safety, e.g., by allowing for increased cancer cell killing (e.g., with higher anti-cancer doses) and reduce toxicity, particularly in subjects with high MARCO expression, e.g., young (e.g., pediatric, adolescent, or young adult) patient populations.

Certain aspects of the disclosure are directed to a combination therapy comprising a first agent and a second agent, wherein the first agent is capable of blocking the interaction between a Macrophage Receptor with Collagenous Structure (MARCO) and a nanoparticle, and the second agent comprises a therapeutic agent (e.g., an anti-cancer agent or an anti-tumor agent), and wherein at least the second agent is in the form of a nanoformulation (also referred to herein as a nanomedicine).

Certain aspects of the disclosure are directed to methods of treating a tumor or cancer in a subject in need thereof comprising administering to the subject a therapeutically effective amount of a combination therapy comprising a first agent and a second agent, wherein the first agent is capable of blocking the interaction between a Macrophage Receptor with Collagenous Structure (MARCO) and a nanoparticle, and the second agent comprises an anti-cancer agent or an anti-tumor agent, and wherein at least the second agent is in the form of a nanoformulation (e.g., a nanoparticle containing the second agent).

Certain aspects of the disclosure are directed to a method for reducing toxicity to a nanoformulated cancer therapy in a subject in need thereof comprising administering to the subject a therapeutically effective amount of a combination therapy comprising a first agent and a second agent, wherein the first agent is capable of blocking the interaction between a Macrophage Receptor with Collagenous Structure (MARCO) and a nanoparticle and the second agent comprises an anti-cancer agent or an anti-tumor agent, wherein at least the second agent is in the form of a nanoformulation.

In some aspects, the method allows for an increase in the dosage or frequency in dosage of the second agent without an increase in toxicity to the subject relative to administering the nanoformulated second agent without the first agent.

In some aspects, the first agent decreases MARCO activity according to one or more of: (i) decreasing the amount of transcription of a MARCO encoding gene, (ii) decreasing the amount of an mRNA or a pre-mRNA encoding a MARCO protein, (iii) decreasing the amount of a MARCO protein, (iv) decreasing the amount of MARCO protein localized to a cell surface, and (v) decreasing the fraction of MARCO which is not bound to a ligand or other binding partner.

In some aspects, the nanoformulation comprises a nanoparticle (e.g. a lipid nanoparticle) or an exosome.

In some aspects, the nanoparticle is selected from the group consisting of organic nanoparticles, inorganic nanoparticles, hybrid nanoparticles, and combinations thereof. In some aspects, the organic nanoparticles are selected from the group consisting of liposome-based nanoparticles, polymer-based nanoparticles, dendrimers, and combinations thereof.

In some aspects, the polymer-based nanoparticles are selected from polymeric nanoparticles, polymeric micelles, and combinations thereof.

In some aspects, prior to administration the subject expresses MARCO at or above a reference MARCO expression level.

In some aspects, the subject is between 1-50 years old. In some aspects, the subject is less than 50 years, less than 45 years, less than 40 years, less than 35 years, or less than 30 years in age.

In some aspects, the first agent comprises a MARCO inhibitor or MARCO blocking agent.

In some aspects, the first agent is a MARCO blocking agent comprising a recombinant MARCO protein or a portion thereof.

In some aspects, the recombinant MARCO protein or the portion thereof comprises: an extracellular domain (ECD) of MARCO, or a scavenger receptor cysteine-rich (SRCR) domain.

In some aspects, the first agent comprises an antibody. In some aspects, the antibody is a monoclonal antibody or a polyclonal antibody.

In some aspects, the first agent comprises an mRNA silencing polynucleotide.

In some aspects, the mRNA silencing polynucleotide is a siRNA, a shRNA, or a miRNA.

In some aspects, the mRNA silencing polynucleotide is a siRNA.

In some aspects, the MARCO activity in the subject after administration of the first agent is at least 1%, 5%, 10%, 15%, 20%, 25%, 30%, 40%, 50%, 60%, 70%, 80%, 90%, 95%, 99%, 99.5%, or 99.9% less than the MARCO activity prior to administration of the first agent.

In some aspects, the cancer is selected from the group consisting of brain cancer, breast cancer, cervical cancer, colorectal cancer, esophageal cancer, lung cancer, non-small cell lung cancer, lymphoma, ovarian cancer, pancreatic cancer, prostate cancer, stomach cancer, thyroid cancer, bladder cancer, and uterine cancer.

In some aspects, the cancer is breast cancer. In some aspects, the cancer is lung cancer. In some aspects, the cancer is a metastatic cancer.

In some aspects, the first agent and the nanoformulated second agent are administered together in a single composition.

In some aspects, the first agent and the nanoformulated second agent are administered in separate compositions.

In some aspects, the second agent comprises a therapeutically effective dosage which is lower than a therapeutically effective dosage of the nanoformulated second agent administered as a monotherapy without the first agent.

In some aspects, the nanoformulated second agent is less toxic to the subject when administered as the combination therapy than as a monotherapy without the first agent. In some aspects, the nanoformulated second agent when administered as the combination therapy is less toxic to the subject's liver and/or kidney than the same dosage of the second agent administered as a monotherapy without the first agent.

In some aspects, the method further comprises detecting the level MARCO in a sample from the subject prior to administration of the combination therapy.

In some aspects, the combination therapy is administered when the MARCO level is high in the subject.

In some aspects, the sample is a blood sample or a urine sample.

In some aspects, the combination therapy is administered systemically to the subject.

In some aspects, the combination therapy is administered to the subject by intravenous, subcutaneous, or intramuscular injection.

In some aspects, the subject has a low tolerance to the nanoformulated second agent when administered as a monotherapy without the first agent.

In some aspects, the second agent is selected from the group consisting of 5-fluorouracil, afatinib, aplidin, azaribine, anastrozole, anthracyclines, axitinib, AVL-101, AVL-291, bendamustine, bleomycin, bortezomib, bosutinib, bryostatin-1, busulfan, calicheamycin, camptothecin, carboplatin, 10-hydroxycamptothecin, carmustine, celecoxib, chlorambucil, cisplatinum, COX-2 inhibitors, irinotecan (CPT-11), SN-38, carboplatin, cladribine, camptothecans, crizotinib, cyclophosphamide, cytarabine, dacarbazine, dasatinib, dinaciclib, docetaxel, dactinomycin, daunorubicin, DM1, DM3, DM4, doxorubicin, 2-pyrrolinodoxorubicine (2-PDox), a pro-drug form of 2-PDox (pro-2-PDox), cyano-morpholino doxorubicin, doxorubicin glucuronide, endostatin, epirubicin glucuronide, erlotinib, estramustine, epidophyllotoxin, erlotinib, entinostat, estrogen receptor binding agents, etoposide (VP16), etoposide glucuronide, etoposide phosphate, exemestane, fingolimod, floxuridine (FUdR), 3′,5′-O-dioleoyl-FudR (FUdR-dO), fludarabine, flutamide, farnesyl-protein transferase inhibitors, flavopiridol, fostamatinib, ganetespib, GDC-0834, GS-1101, gefitinib, gemcitabine, hydroxyurea, ibrutinib, idarubicin, idelalisib, ifosfamide, imatinib, lapatinib, lenolidamide, leucovorin, LFM-A13, lomustine, mechlorethamine, melphalan, mercaptopurine, 6-mercaptopurine, methotrexate, mitoxantrone, mithramycin, mitomycin, mitotane, monomethylauristatin F (MMAF), monomethylauristatin D (MMAD), monomethylauristatin E (MMAE), navelbine, neratinib, nilotinib, nitrosurea, olaparib, plicomycin, procarbazine, paclitaxel, PCI-32765, pentostatin, PSI-341, raloxifene, semustine, SN-38, sorafenib, streptozocin, SU11248, sunitinib, tamoxifen, temazolomide, transplatinum, thalidomide, thioguanine, thiotepa, teniposide, topotecan, uracil mustard, vatalanib, vinorelbine, vinblastine, vincristine, vinca alkaloids, ZD1839, and any combination thereof.

In some aspects, the second agent comprises paclitaxel.

In some aspects, the second agent comprises doxorubicin.

In some aspects, the second agent comprises irinotecan.

In some aspects, the second agent comprises albumin-bound paclitaxel (also referred to herein as nab-paclitaxel).

In some aspects, the second agent comprises PEGylated liposomal doxorubicin.

In some aspects, the second agent comprises liposomal irinotecan.

Certain aspects of the disclosure are directed to a kit comprising a first agent and a nanoformulated second agent, wherein the first agent and the nanoformulated second agent can be combined to produce a combination therapy, wherein the first agent is capable of blocking the interaction between a Macrophage Receptor with Collagenous Structure (MARCO) and a nanoparticle, and the nanoformulated second agent comprises an anti-cancer agent or an anti-tumor agent.

In some aspects, the nanoformulated second agent is capable of being metabolized in the liver of a/the subject.

In some aspects, the second agent is a chemotherapeutic agent or a polynucleotide. In some aspects, the second agent is a polynucleotide, and wherein the polynucleotide is an RNA molecule or a DNA molecule.

In some aspects, the second agent comprises paclitaxel, doxorubicin, irinotecan, albumin-bound paclitaxel, PEGylated liposomal doxorubicin, liposomal irinotecan, or any combination thereof.

In some aspects, the first agent is a/the MARCO inhibitor or MARCO blocking agent.

In some aspects, the first agent is selected from the group consisting of a/the recombinant MARCO protein or a portion thereof comprising an extracellular domain (ECD) of MARCO and/or a scavenger receptor cysteine-rich (SRCR) domain; an/the antibody; and a/the mRNA silencing polynucleotide.

### DETAILED DESCRIPTION OF THE DISCLOSURE

### I. Definitions

In order to further define this disclosure, the following terms and definitions are provided.

The singular forms “a,” “an” and “the” include plural referents unless the context clearly dictates otherwise. The terms “a” (or “an”), as well as the terms “one or more,” and “at least one” can be used interchangeably herein. In certain aspects, the term “a” or “an” means “single.” In other aspects, the term “a” or “an” includes “two or more” or “multiple.”

The term “about” is used herein to mean approximately, roughly, around, or in the regions of. When the term “about” is used in conjunction with a numerical range, it modifies that range by extending the boundaries above and below the numerical values set forth. In general, the term “about” is used herein to modify a numerical value above and below the stated value by a variance of 10 percent, up or down (higher or lower).

Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 or “between 1-6” should be considered to have specifically disclosed sub-ranges such as from 1 to 3, from 1 to 4, from 1 to 5, from 2 to 4, from 2 to 6, from 3 to 6, etc., as well as individual numbers within that range, for example, 1, 2, 3, 4, 5, and 6. This applies regardless of the breadth of the range. Numeric ranges recited are inclusive of the numbers defining the range and include each integer within the defined range.

Units, prefixes, and symbols are denoted in their Système International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the disclosure. Thus, ranges recited herein are understood to be shorthand for all of the values within the range, inclusive of the recited endpoints. For example, a range of 1 to 10 is understood to include any number, combination of numbers, or sub-range from the group consisting of 1, 2, 3, 4, 5, 6, 7, 8, 9, and 10.

Where a value is explicitly recited, it is to be understood that values which are about the same quantity or amount as the recited value are also within the scope of the disclosure. Where a combination is disclosed, each subcombination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The term “pharmaceutically acceptable” as used herein refers to those compounds, materials, compositions, formulations, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “excipient” refers to any substance, not itself a therapeutic agent, which may be used in a composition for delivery of an active therapeutic agent to a subject or combined with an active therapeutic agent (e.g., to create a pharmaceutical composition) to improve its handling or storage properties or to permit or facilitate formation of a dose unit of the composition (e.g., formation of a hydrogel which may then be optionally incorporated into a patch). Excipients include, but are not limited to, solvents, penetration enhancers, wetting agents, antioxidants, lubricants, emollients, substances added to improve appearance or texture of the composition and substances used to form hydrogels. Any such excipients can be used in any dosage forms according to the present disclosure. The foregoing classes of excipients are not meant to be exhaustive but merely illustrative as a person of ordinary skill in the art would recognize that additional types and combinations of excipients could be used to achieve the desired goals for delivery of a drug. The excipient can be an inert substance, an inactive substance, and/or a not medicinally active substance. The excipient can serve various purposes. A person skilled in the art can select one or more excipients with respect to the particular desired properties by routine experimentation and without any undue burden. The amount of each excipient used can vary within ranges conventional in the art. Techniques and excipients which can be used to formulate dosage forms are described in Handbook of Pharmaceutical Excipients, 6th edition, Rowe et al., Eds., American Pharmaceuticals Association and the Pharmaceutical Press, publications department of the Royal Pharmaceutical Society of Great Britain (2009); and Remington: the Science and Practice of Pharmacy, 21th edition, Gennaro, Ed., Lippincott Williams & Wilkins (2005).

In some aspects, as used herein, a “first agent” refers to an agent which decreases MARCO-mediated macrophage uptake of a second agent.

In some aspects, as used herein, a “second agent” refers to a therapeutic agent which is nanoformulated (also referred to herein as a nanomedicine).

The term “effective amount” or “pharmaceutically effective amount” or “therapeutically effective amount” as used herein refers to the amount or quantity of a drug or pharmaceutically active substance which is sufficient to elicit the required or desired therapeutic response, or in other words, the amount which is sufficient to elicit an appreciable biological response when administered to a patient. In some aspects, as used in the context of a combination therapy, an effective amount or pharmaceutically effective amount or therapeutically effective amount can refer to an amount of a second agent sufficient to achieve a therapeutic effect, e.g., following administration of a first agent.

The term “treating” or “treatment” as used herein refers to the administration of a composition to a subject for therapeutic purposes.

As used herein, the term “pediatric patient” means a human subject 18 years old or younger. As used herein, the term “adolescent patient” means a human subject 10-19 years old. As used herein, the term “young adult patient” means a human subject 18-25 years old. As used herein, the term “middle aged adult” means a human subject 40-60 years old. For example, when referring to a subject who is younger than a middle aged adult, the subject is younger than 40 years in age.

As used herein, the term “nanoparticle” means a particle having a diameter 1 nm or greater and less than 1 μm, and wherein the particle comprises an active pharmaceutical ingredient. A nanoparticle of the present disclosure can comprise an organic nanoparticle (optionally, a liposome-based nanoparticle, a polymer-based nanoparticle, a dendrimer, or any combination thereof), an inorganic nanoparticle, a hybrid nanoparticle, and any combinations thereof. In some aspects, the nanoparticle is a lipid nanoparticle. In some aspects, the nanoparticle is a polymer-based nanoparticle (optionally, a polymeric nanoparticle, a polymeric micelle, or any combination thereof). As used herein, the term “nanoformulation,” and the term “nanoformulated” when referring to a drug (e.g., “nanoformulated drug”), therapeutic agent (e.g., “nanoformulated therapeutic agents,” “nanoformulated second agent”), or therapy (e.g., “nanoformulated cancer therapy”) means a composition comprising a nanoparticle of the present disclosure, wherein the composition is suitable for therapeutic administration to a subject in need thereof.

As used herein, the term “combination therapy” means a therapy that includes at least a first agent and a second agent, which can be administered together or separately. In some aspects, a first agent is capable of blocking the interaction between a Macrophage Receptor with Collagenous Structure (MARCO) and a nanoparticle, and a second agent is in the form of a nanoformulation (e.g., encapsulated in a nanoparticle). In some aspects, the first agent of the combination therapy comprises a MARCO inhibitor or MARCO blocking agent. In some aspects, the first agent and the nanoformulated second agent of the combination therapy are formulated together in a single composition or as separate compositions.

As used herein, the term “Macrophage Receptor with Collagenous Structure” or its abbreviation “MARCO” means a scavenger receptor class-A protein expressed on the cell surface of macrophages that mediates opsonin-independent phagocytosis. An exemplary sequence for MARCO from Homo sapiens can be found at UniProt Accession Number Q9UEW3 (uniprot.org/uniprot/Q9UEW3). It is understood that there are at least two isoforms known for MARCO, resulting from splice variants. The isoform identified as canonical is Q9UEW3-1. The Q9UEW3-2 isoform is identified as missing amino acids 1-78 of the canonical (Q9UEW3-1) sequence. The present disclosure encompasses a first agent which is suitable for inhibiting or decreasing production or activity of the Q9UEW3-1 or the Q9UEW3-2 isoform of MARCO. In some aspects, the present disclosure is directed to inhibiting or decreasing the production or activity of the Q9UEW3-1 isoform.

As used herein, the term “low,” when referring to MARCO expression or levels (e.g., “the MARCO level is low in the subject,” or “low MARCO expression”), means <25% of a subject's peripheral blood mononuclear cell (PBMC) CD11b⁺ cells are estimated or determined to be positive for MARCO, using standard laboratory techniques.

As used herein, the term “high,” when referring to MARCO expression or levels (e.g., “the MARCO level is high in the subject,” or “high MARCO expression”), means≥25% of a subject's peripheral blood mononuclear cell (PBMC) CD11b⁺ cells are estimated or determined to be positive for MARCO, using standard laboratory techniques.

It is understood that wherever aspects are described herein with the language “comprising,” otherwise analogous aspects described in terms of “consisting of” and/or “consisting essentially of” are also provided.

It is to be understood that headers are provided solely for ease of reading, and are not intended to be limiting. Aspects disclosed under one or more headers can be applicable to or combinable with aspects disclosed under one or more other headers.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

### II. Marco Blocking Agents

Certain aspects of the disclosure are directed to compositions and methods comprising an agent (e.g. a MARCO blocking agent, sometimes referred to herein as a first agent) capable of blocking the interaction between a Macrophage Receptor with Collagenous Structure (MARCO) and a nanoparticle. In some aspects, the first agent can improve uptake of a nanoformulated therapeutic agent (e.g., an anti-cancer and/or anti-tumor agent, sometimes referred to herein as a second agent).

In some aspects, the blocking the MARCO-nanoparticles interaction, e.g., using recombinant MARCO protein (the ligand-binding SRCR domain) or anti-MARCO monoclonal antibody, decreases the nanoparticle uptake by macrophages.

In some aspects, the first agent decreases MARCO activity according to one or more of: (i) decreasing the amount of transcription of a MARCO encoding gene, (ii) decreasing the amount of an mRNA or a pre-mRNA encoding a MARCO protein, (iii) decreasing the amount of a MARCO protein, (iv) decreasing the amount of MARCO protein localized to a cell surface, and (v) decreasing the fraction of MARCO which is not bound to a ligand or other binding partner.

In some aspects, the MARCO blocking agent (e.g., the first agent) is a MARCO inhibitor.

In some aspects, the MARCO blocking agent (e.g., the first agent) is selected from the group consisting of anti-MARCO antibodies (e.g., monoclonal ED31, PLK-1, ABN 1389), anti-MARCO ScFv, Fab, Fab′, and Fab2, anti-MARCO ScFv mRNA, anti-MARCO miRNA, MARCO antisense RNA, MARCO siRNA, anti-MARCO DNA, anti-MARCO oligonucleotides, anti-MARCO peptide inhibitors, or any combinations thereof.

In some aspects, the MARCO blocking agent (e.g., the first agent) is an anti-MARCO antibody. In some aspects, the antibody is a monoclonal antibody or a polyclonal antibody.

In some aspects, the MARCO blocking agent (e.g., the first agent) is a recombinant MARCO protein (the ligand-binding SRCR domain).

In some aspects, the MARCO blocking agent (e.g., the first agent) comprises an mRNA silencing polynucleotide.

In some aspects, the MARCO blocking agent (e.g., the first agent) comprises a mRNA silencing polynucleotide selected from a siRNA, a shRNA, or a miRNA. In some aspects the mRNA silencing polynucleotide is a siRNA.

In some aspects, a nanoformulation disclosed herein comprises a therapeutic agent (e.g., an anti-cancer and/or anti-tumor agent, sometimes referred to herein as a second agent) disclosed herein contained in a nano-carrier (e.g., nanoparticle) disclosed herein.

In some aspects, the nanoformulation comprising the therapeutic agent (e.g., an anti-cancer and/or anti-tumor agent, sometimes referred to herein as a second agent) contained in a nano-carrier (e.g., nanoparticle) disclosed herein is co-administered (together or in separate compositions) as part of a combination therapy with a MARCO blocking agent disclosed herein.

### III. Nanoformulation

Certain aspects of the disclosure are directed to compositions and methods improved uptake of a therapeutic agent (e.g., an anti-cancer and/or anti-tumor agent, sometimes referred to herein as a second agent) contained in a nano-carrier (e.g., an exosome or a nanoparticle), which is administered in combination with an agent that is capable of blocking binding of MARCO to the nanoparticle disclosed herein.

In some aspects, the nanoformulation comprises an exosome.

In some aspects, the nanoformulation comprises a nanoparticle (e.g., a lipid nanoparticle or a protein nanoparticle).

In some aspects, the nanoparticles disclosed herein can have specific sizes, shapes, and surface characteristics that influence on the efficiency of the nanoformulated therapeutic agent delivery and efficacy.

In some aspects, the nanoparticle comprises a diameter range of 1 nm to 200 nm, 5 nm to 200 nm, 5 nm to 150 nm, 10 nm to 150 nm, 10 nm to 100 nm, or 10 nm to 75 nm. In some aspects, the nanoparticle has a diameter 10 nm to 100 nm. In some aspects, the nanoparticle has a diameter 10 to 200 nm. In some aspects, the nanoparticle has a diameter 50 to 200 nm. In some aspects, the nanoparticle has a diameter 100 to 200 nm.

In some aspects, the nanoparticle can be coated with hydrophilic materials such as polyethylene glycol (PEG), e.g., to lessen the opsonization and therefore help avoid clearance by the immune system. In some aspects, the nanoparticles can be modified to become hydrophilic, e.g. to increase the time period of drugs in circulation and enhances their penetration and accumulation in tumors. Is some aspects, the time period of nanoparticles in circulation is increased following MARCO blockade.

In some aspects, the nanoparticle is selected from the group consisting of organic nanoparticles, inorganic nanoparticles, hybrid nanoparticles, and combinations thereof.

In some aspects, the organic nanoparticles are selected from the group consisting of liposome-based nanoparticles, polymer-based nanoparticles, dendrimers, and combinations thereof.

In some aspects, the polymer-based nanoparticles are selected from polymeric nanoparticles, polymeric micelles, and combinations thereof.

In some aspects, the inorganic nanoparticles are selected from the group consisting of gold NPs (Au NPs), carbon nanotubes, silica NPs, magnetic NPs, quantum dots, and combinations thereof.

In some aspects, the hybrid nanoparticles combine the advantages of different NPs, including lipid-polymer hybrid NPs, organic-inorganic hybrid NPs, and/or cell membrane-coated NPs.

In some aspects, the nanoformulation comprises a nano-carrier (e.g., nanoparticle) disclosed herein contains a therapeutic agent (e.g., an anti-cancer and/or anti-tumor agent, sometimes referred to herein as a second agent).

In some aspects, the nanoformulation comprising a nano-carrier (e.g., nanoparticle) disclosed herein containing a therapeutic agent (e.g., an anti-cancer and/or anti-tumor agent, sometimes referred to herein as a second agent) is co-administered (together or in separate compositions) as part of a combination therapy with a MARCO blocking agent disclosed herein.

### IV. Anti-Cancer/Anti-Tumor Agents

Certain aspects of the disclosure are directed to compositions and methods for improved uptake of a nanoformulated therapeutic agent (e.g., an anti-cancer and/or anti-tumor agent, sometimes referred to herein as a second agent).

In some aspects, the nanoformulated therapeutic agent (e.g., the second agent) comprises a therapeutic agent which has been or is approved for use in a human population by the United States Food and Drug Administration or another regulatory authority in the United States or another jurisdiction. Non-limiting examples of suitable therapeutic agents (e.g., the second agent) are shown in Tables 1-3 of Ventola CL. 2012. The nanomedicine revolution: part 2: current and future clinical applications. P & T: a Peer-reviewed Journal for Formulary Management. 37 (10): 582-591; and Table 2 of Soares, S., et al. 2018. Nanomedicine: Principles, Properties, and Regulatory Issues. Front. Chem. 6:360.

In some aspects, the nanoformulated therapeutic agent (e.g., the second agent) comprises a chemotherapeutic agent.

In some aspects, the anti-cancer and/or anti-tumor agent (e.g., second agent) comprises one or more of 5-fluorouracil, afatinib, aplidin, azaribine, anastrozole, anthracyclines, axitinib, AVL-101, AVL-291, bendamustine, bleomycin, bortezomib, bosutinib, bryostatin-1, busulfan, calicheamycin, camptothecin, carboplatin, 10-hydroxycamptothecin, carmustine, celecoxib, chlorambucil, cisplatinum, COX-2 inhibitors, irinotecan (CPT-11), SN-38, carboplatin, cladribine, camptothecans, crizotinib, cyclophosphamide, cytarabine, dacarbazine, dasatinib, dinaciclib, docetaxel, dactinomycin, daunorubicin, DM1, DM3, DM4, doxorubicin, 2-pyrrolinodoxorubicine (2-PDox), a pro-drug form of 2-PDox (pro-2-PDox), cyano-morpholino doxorubicin, doxorubicin glucuronide, endostatin, epirubicin glucuronide, erlotinib, estramustine, epidophyllotoxin, erlotinib, entinostat, estrogen receptor binding agents, etoposide (VP16), etoposide glucuronide, etoposide phosphate, exemestane, fingolimod, floxuridine (FUdR), 3′,5′-O-dioleoyl-FudR (FUdR-dO), fludarabine, flutamide, farnesyl-protein transferase inhibitors, flavopiridol, fostamatinib, ganetespib, GDC-0834, GS-1101, gefitinib, gemcitabine, hydroxyurea, ibrutinib, idarubicin, idelalisib, ifosfamide, imatinib, lapatinib, lenolidamide, leucovorin, LFM-A13, lomustine, mechlorethamine, melphalan, mercaptopurine, 6-mercaptopurine, methotrexate, mitoxantrone, mithramycin, mitomycin, mitotane, monomethylauristatin F (MMAF), monomethylauristatin D (MMAD), monomethylauristatin E (MMAE), navelbine, neratinib, nilotinib, nitrosurea, olaparib, plicomycin, procarbazine, paclitaxel, PCI-32765, pentostatin, PSI-341, raloxifene, semustine, SN-38, sorafenib, streptozocin, SU11248, sunitinib, tamoxifen, temazolomide, transplatinum, thalidomide, thioguanine, thiotepa, teniposide, topotecan, uracil mustard, vatalanib, vinorelbine, vinblastine, vincristine, vinca alkaloids and ZD1839.

In some aspects, the anti-cancer and/or anti-tumor agent (e.g., second agent) comprises paclitaxel. In some aspects, the anti-cancer and/or anti-tumor agent (e.g., second agent) comprises albumin-bound paclitaxel. In some aspects, the second agent comprises ABRAXANE®.

In some aspects, the anti-cancer and/or anti-tumor agent (e.g., second agent) comprises doxorubicin. In some aspects, the anti-cancer and/or anti-tumor agent (e.g., second agent) comprises PEGylated liposomal doxorubicin. In some aspects, the second agent comprises DOXIL®.

In some aspects, the anti-cancer and/or anti-tumor agent (e.g., second agent) comprises irinotecan. In some aspects, the anti-cancer and/or anti-tumor agent (e.g., second agent) comprises liposomal irinotecan. In some aspects, the second agent comprises ONIVYDE®.

In some aspects, the anti-cancer and/or anti-tumor agent (e.g., second agent) is a polynucleotide. In some aspects, the polynucleotide is an RNA molecule or a DNA molecule. In some aspects, the polynucleotide comprises a stabilizing chemical modification. In some aspects, the polynucleotide comprises one or more phosphorothioate internucleoside linkages.

In some aspects, the anti-cancer and/or anti-tumor agent (e.g., second agent) is an antibody.

In some aspects, a nano-carrier (e.g., nanoparticle) disclosed herein contains a therapeutic agent (e.g., an anti-cancer and/or anti-tumor agent, sometimes referred to herein as a second agent) disclosed herein.

In some aspects, a nano-carrier (e.g., nanoparticle) disclosed herein contains a therapeutic agent (e.g., an anti-cancer and/or anti-tumor agent, sometimes referred to herein as a second agent) disclosed herein, which is co-administered (together or in separate compositions) as part of a combination therapy with a MARCO blocking agent disclosed herein.

### V. Combination Therapy

Certain aspects of the disclosure are directed to a combination therapy comprising a first agent and a second agent, wherein the first agent is capable of blocking the interaction between a Macrophage Receptor with Collagenous Structure (MARCO) and a nanoparticle, and the second agent comprises a therapeutic agent (e.g., an anti-cancer agent or an anti-tumor agent), and wherein at least the second agent is in the form of a nanoformulation.

In some aspects, the nanoformulation of the combination therapy comprises an exosome.

In some aspects, the nanoformulation of the combination therapy comprises a nanoparticle.

In some aspects, the nanoformulation of the combination therapy comprises a lipid nanoparticle.

In some aspects, the nanoformulation of the combination therapy comprises a protein nanoparticle.

In some aspects, the first agent of the combination therapy is capable of decreasing MARCO activity according to one or more of: (i) decreasing the amount of transcription of a MARCO encoding gene, (ii) decreasing the amount of an mRNA or a pre-mRNA encoding a MARCO protein, (iii) decreasing the amount of a MARCO protein, (iv) decreasing the amount of MARCO protein localized to a cell surface, and (v) decreasing the fraction of MARCO which is not bound to a ligand or other binding partner.

In some aspects, the first agent of the combination therapy comprises a MARCO inhibitor or MARCO blocking agent.

In some aspects, the first agent of the combination therapy is a MARCO blocking agent comprising a recombinant MARCO protein or a portion thereof.

In some aspects, the first agent of the combination therapy is a recombinant MARCO protein or the portion thereof comprising: an extracellular domain (ECD) of MARCO, or a scavenger receptor cysteine-rich (SRCR) domain.

In some aspects, the first agent of the combination therapy comprises an antibody. In some aspects, the antibody is a monoclonal antibody or a polyclonal antibody.

In some aspects, the first agent of the combination therapy comprises an mRNA silencing polynucleotide. In some aspects, the mRNA silencing polynucleotide is a siRNA, a shRNA, or a miRNA. In some aspects, the mRNA silencing polynucleotide is a siRNA.

In some aspects, the first agent and the nanoformulated second agent of the combination therapy are formulated together in a single composition.

In some aspects, the first agent and the nanoformulated second agent of the combination therapy are formulated as separate compositions.

In some aspects, the second agent can be administered at a therapeutically effective dosage which is lower than a therapeutically effective dosage of the nanoformulated second agent administered as a monotherapy without the first agent.

In some aspects, the nanoformulated second agent is less toxic to the subject when administered as the combination therapy than as a monotherapy without the first agent.

In some aspects, the combination therapy is formulated for intravenous, subcutaneous, or intramuscular injection.

In some aspects, the second agent of the combination therapy is selected from the group consisting of 5-fluorouracil, afatinib, aplidin, azaribine, anastrozole, anthracyclines, axitinib, AVL-101, AVL-291, bendamustine, bleomycin, bortezomib, bosutinib, bryostatin-1, busulfan, calicheamycin, camptothecin, carboplatin, 10-hydroxycamptothecin, carmustine, celecoxib, chlorambucil, cisplatinum, COX-2 inhibitors, irinotecan (CPT-11), SN-38, carboplatin, cladribine, camptothecans, crizotinib, cyclophosphamide, cytarabine, dacarbazine, dasatinib, dinaciclib, docetaxel, dactinomycin, daunorubicin, DM1, DM3, DM4, doxorubicin, 2-pyrrolinodoxorubicine (2-PDox), a pro-drug form of 2-PDox (pro-2-PDox), cyano-morpholino doxorubicin, doxorubicin glucuronide, endostatin, epirubicin glucuronide, erlotinib, estramustine, epidophyllotoxin, erlotinib, entinostat, estrogen receptor binding agents, etoposide (VP16), etoposide glucuronide, etoposide phosphate, exemestane, fingolimod, floxuridine (FUdR), 3′,5′-O-dioleoyl-FudR (FUdR-dO), fludarabine, flutamide, farnesyl-protein transferase inhibitors, flavopiridol, fostamatinib, ganetespib, GDC-0834, GS-1101, gefitinib, gemcitabine, hydroxyurea, ibrutinib, idarubicin, idelalisib, ifosfamide, imatinib, lapatinib, lenolidamide, leucovorin, LFM-A13, lomustine, mechlorethamine, melphalan, mercaptopurine, 6-mercaptopurine, methotrexate, mitoxantrone, mithramycin, mitomycin, mitotane, monomethylauristatin F (MMAF), monomethylauristatin D (MMAD), monomethylauristatin E (MMAE), navelbine, neratinib, nilotinib, nitrosurea, olaparib, plicomycin, procarbazine, paclitaxel, PCI-32765, pentostatin, PSI-341, raloxifene, semustine, SN-38, sorafenib, streptozocin, SU11248, sunitinib, tamoxifen, temazolomide, transplatinum, thalidomide, thioguanine, thiotepa, teniposide, topotecan, uracil mustard, vatalanib, vinorelbine, vinblastine, vincristine, vinca alkaloids, ZD1839, and any combination thereof.

In some aspects, the second agent of the combination therapy comprises paclitaxel.

In some aspects, the second agent of the combination therapy comprises doxorubicin.

In some aspects, the second agent of the combination therapy comprises irinotecan.

In some aspects, the second agent of the combination therapy comprises albumin-bound paclitaxel (also referred to herein as nab-paclitaxel).

In some aspects, the second agent of the combination therapy comprises PEGylated liposomal doxorubicin.

In some aspects, the present disclosure provides a combination therapy comprising a first agent and a second agent, wherein the first agent is an agent which reduces a Macrophage Receptor With Collagenous Structure (MARCO) activity relative to a reference level of the MARCO activity, wherein the second agent is an anti-cancer agent or an anti-tumor agent, wherein at least the second agent is in the form of a nanoformulation, and wherein the first agent and the second agent are formulated at concentrations which are therapeutically effective for the treatment of a tumor or a cancer upon administration of the combination therapy to the subject.

In some aspects, the present disclosure provides a combination therapy comprising a first agent and a second agent, wherein the first agent is a Macrophage Receptor With Collagenous Structure (MARCO) inhibitor, which reduces MARCO activity in the subject, wherein the second agent is a nanoformulated cancer therapy, wherein the nanoformulated cancer therapy is an anti-cancer agent or an anti-tumor agent, and wherein the first agent and the second agent are formulated at concentrations which are therapeutically effective for modifying (e.g., increasing) the tolerance to the nanoformulated cancer therapy in a subject upon administration of the combination therapy to the subject.

In some aspects, the increased tolerance comprises an increase in the dosage or frequency in dosage of the second agent without an increase in toxicity to the subject.

In some aspects, the MARCO activity is one or more of: an amount of transcription of a MARCO encoding gene, an amount of an mRNA or a pre-mRNA encoding a MARCO protein, an amount of a MARCO protein, an amount of MARCO protein localized to a cell surface, and a fraction of MARCO which is not bound to a ligand or other binding partner.

In some aspects, the nanoformulation or the nanoformulated cancer therapy comprises an exosome.

In some aspects, the nanoformulation or the nanoformulated cancer therapy comprises a nanoparticle. In some aspects, the nanoformulation or the nanoformulated cancer therapy comprises a lipid nanoparticle. In some aspects, the nanoformulation or the nanoformulated cancer therapy comprises a protein nanoparticle.

In some aspects, the subject expresses MARCO at or above a reference MARCO expression level. In some aspects, the reference MARCO expression level is a level of MARCO in the subject prior to administration of the combination therapy. In some aspects, the reference MARCO expression level is a level of MARCO in a reference subject. In some aspects, the reference subject is the same age as the subject. In some aspects, the reference subject is within 1, 2, 3, 4, 5, 6, 7, 8, 9, 10, 11, 12, 13, 14, or 15 years of the age of the subject.

In some aspects, the subject is less than 50 years, less than 45 years, less than 40 years, less than 35 years, or less than 30 years in age. In some aspects, the subject is less than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 years in age.

In some aspects, the subject is between 1-50 years old. In some aspects, the subject is between 1-10 years old, 1-20 years old, 1-30 years old, 1-40 years old, 1-50 years old, 1-60 years old, 1-70 years old, 1-80 years old, or 1-90 years old. In some aspects, the subject is between 5-10 years old, 5-20 years old, 5-30 years old, 5-40 years old, 5-50 years old, 10-20 years old, 10-30 years old, 10-40 years old, or 10-50 years old.

In some aspects, the subject is a pediatric patient, an adolescent patient, or a young adult patient. In some aspects, the subject is younger than a middle aged adult.

In some aspects, the MARCO activity in the subject after administration of the first agent is at least 1%, 5%, 10%, 15%, 20%, 25%, 30%, 40%, 50%, 60%, 70%, 80%, 90%, 95%, 99%, 99.5%, or 99.9% less than the MARCO activity prior to administration of the first agent. In some aspects, the MARCO activity in the subject after administration of the first agent is at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 99.99% less than the MARCO activity prior to administration of the first agent.

In some aspects, the cancer is selected from the group consisting of brain cancer, breast cancer, cervical cancer, colorectal cancer, esophageal cancer, lung cancer, non-small cell lung cancer, lymphoma, ovarian cancer, pancreatic cancer, prostate cancer, stomach cancer, thyroid cancer, bladder cancer, and uterine cancer. In some aspects, the cancer is breast cancer. In some aspects, the cancer is lung cancer. In some aspects, the cancer is a metastatic cancer.

In some aspects, the first agent and the second agent are administered together in a composition.

In some aspects, the first agent and the second agent are administered in separate compositions.

In some aspects, the second agent is therapeutically effective at a dosage which is lower than a therapeutically effective dosage of the second agent administered as a monotherapy.

In some aspects, the second agent is less toxic to the subject when administered as the combination therapy than as a monotherapy without the first agent.

In some aspects, the second agent is administered at a dosage which is less toxic to the subject's liver or kidney than a therapeutically effective dosage of the second agent administered as a monotherapy.

In some aspects, the combination therapy is formulated for systemic administration. In some aspects, the combination therapy is formulated for administration to the subject by parenteral injection. In some aspects, the combination therapy is formulated for administration to the subject by intravenous, subcutaneous, or intramuscular injection.

Where aspects describe the treatment of or combination therapies for use in treating a cancer or tumor, it is to be understood that those aspects can be combined with other aspects, and a reference to a cancer or tumor can be replaced with a reference to another disease or condition. Where aspects describe the treatment of or combination therapies for use in treating a cancer or tumor with an anticancer or antitumor agent, it is to be understood that those aspects can be combined with other aspects, and a reference to an anticancer or antitumor agent can be replaced with a reference to an agent suitable for treating another disease or condition.

### VI. Methods of Treatment/Use

Certain aspects of the disclosure are directed to methods of treating a disease (e.g., a tumor or cancer) in a subject in need thereof comprising administering to the subject a therapeutically effective amount of a combination therapy comprising a first agent and a second agent, wherein the first agent is capable of blocking the interaction between a Macrophage Receptor with Collagenous Structure (MARCO) and a nanoparticle, and the second agent comprises an anti-cancer agent or an anti-tumor agent, and wherein at least the second agent is in the form of a nanoformulation (e.g., a nanoparticle containing the second agent).

Certain aspects of the disclosure are directed to a method for reducing toxicity to a nanoformulated therapy (e.g., cancer therapy) in a subject in need thereof comprising administering to the subject a therapeutically effective amount of a combination therapy comprising a first agent and a second agent, wherein the first agent is capable of blocking the interaction between a Macrophage Receptor with Collagenous Structure (MARCO) and a nanoparticle and the second agent comprises a therapeutic agent (e.g., an anti-cancer agent or an anti-tumor agent), wherein at least the second agent is in the form of a nanoformulation.

In some aspects, the present disclosure provides a method of treating a tumor or cancer in a subject in need thereof comprising administering to the subject a therapeutically effective amount of a combination therapy comprising a first agent and a second agent, wherein the first agent is a Macrophage Receptor With Collagenous Structure (MARCO) inhibitor, which reduces MARCO activity in the subject, wherein the second agent is an anti-cancer agent or an anti-tumor agent, and wherein at least the second agent is in the form of a nanoformulation.

In some aspects, the present disclosure provides a method for modifying (e.g., increasing) of the subject tolerance to a nanoformulated cancer therapy in a subject in need thereof comprising administering to the subject a therapeutically effective amount of a first agent in combination with administering a second agent comprising the nanoformulated cancer therapy, wherein the first agent is a Macrophage Receptor With Collagenous Structure (MARCO) inhibitor, which reduces MARCO activity in the subject, wherein the nanoformulated cancer therapy is an anti-cancer agent or an anti-tumor agent.

In some aspects, the method allows for modifying in the dosage or frequency in dosage of the second agent without an increase in toxicity to the subject relative to administering the nanoformulated second agent without the first agent. In some aspects, the increased tolerance comprises an increase in the dosage or frequency in dosage of the second agent without an increase in toxicity to the subject.

In some aspects, the MARCO activity is one or more of: an amount of transcription of a MARCO encoding gene, an amount of an mRNA or a pre-mRNA encoding a MARCO protein, an amount of a MARCO protein, an amount of MARCO protein localized to a cell surface, and a fraction of MARCO which is not bound to a ligand or other binding partner. In some aspects, the first agent decreases MARCO activity according to one or more of: (i) decreasing the amount of transcription of a MARCO encoding gene, (ii) decreasing the amount of an mRNA or a pre-mRNA encoding a MARCO protein, (iii) decreasing the amount of a MARCO protein, (iv) decreasing the amount of MARCO protein localized to a cell surface, and (v) decreasing the fraction of MARCO which is not bound to a ligand or other binding partner.

In some aspects, prior to administration the subject expresses MARCO at or above a reference MARCO expression level.

In some aspects, the nanoformulation comprises an exosome.

In some aspects, the nanoformulation comprises a nanoparticle. In some aspects, the nanoformulation comprises a lipid nanoparticle.

In some aspects, the subject expresses MARCO at or above a reference MARCO expression level. In some aspects, the subject expressed MARCO above a low level of MARCO expression. In some aspects, the reference MARCO expression level is a level of MARCO in the subject prior to administration of the combination therapy. In some aspects, the reference MARCO expression level is a level of MARCO in a reference subject. In some aspects, the reference subject is the same age as the subject. In some aspects, the reference subject is within 1, 2, 3, 4, 5, 6, 7, 8, 9, 10, 11, 12, 13, 14, or 15 years of the age of the subject.

In some aspects, the subject is less than 50 years, less than 45 years, less than 40 years, less than 35 years, or less than 30 years in age. In some aspects, the subject is less than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 years in age.

In some aspects, the subject is between 1-50 years old. In some aspects, the subject is between 1-10 years old, 1-20 years old, 1-30 years old, 1-40 years old, 1-50 years old, 1-60 years old, 1-70 years old, 1-80 years old, or 1-90 years old. In some aspects, the subject is between 5-10 years old, 5-20 years old, 5-30 years old, 5-40 years old, 5-50 years old, 10-20 years old, 10-30 years old, 10-40 years old, or 10-50 years old.

In some aspects, the subject is between 1-50 years old. In some aspects, the subject is less than 50 years, less than 45 years, less than 40 years, less than 35 years, or less than 30 years in age.

In some aspects, the subject is a pediatric patient, an adolescent patient, or a young adult patient. In some aspects, the subject is younger than a middle aged adult.

In some aspects, the first agent comprises a MARCO inhibitor or MARCO blocking agent. In some aspects, the first agent is a MARCO blocking agent comprising a recombinant MARCO protein or a portion thereof. In some aspects, the recombinant MARCO protein or the portion thereof comprises: an extracellular domain (ECD) of MARCO, or a scavenger receptor cysteine-rich (SRCR) domain.

In some aspects, the first agent comprises an antibody. In some aspects, antibody is a monoclonal antibody or a polyclonal antibody.

In some aspects, the first agent comprises an mRNA silencing polynucleotide. In some aspects, the mRNA silencing polynucleotide is an siRNA, an shRNA, or an miRNA. In some aspects, the mRNA silencing polynucleotide is an siRNA.

In some aspects, the MARCO activity in the subject after administration of the first agent is at least 1%, 5%, 10%, 15%, 20%, 25%, 30%, 40%, 50%, 60%, 70%, 80%, 90%, 95%, 99%, 99.5%, or 99.9% less than the MARCO activity prior to administration of the first agent. In some aspects, the MARCO activity in the subject after administration of the first agent is at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 99.99% less than the MARCO activity prior to administration of the first agent.

In some aspects, the cancer is selected from the group consisting of brain cancer, breast cancer, cervical cancer, colorectal cancer, esophageal cancer, lung cancer, non-small cell lung cancer, lymphoma, ovarian cancer, pancreatic cancer, prostate cancer, stomach cancer, thyroid cancer, bladder cancer, and uterine cancer. In some aspects, the cancer is breast cancer. In some aspects, the cancer is lung cancer. In some aspects, the cancer is a metastatic cancer.

In some aspects, the first agent and the second agent are administered together in a composition.

In some aspects, the first agent and the second agent are administered in separate compositions.

In some aspects, the second agent is therapeutically effective at a dosage which is lower than a therapeutically effective dosage of the second agent administered as a monotherapy.

In some aspects, the second agent is less toxic to the subject when administered as the combination therapy than as a monotherapy without the first agent.

In some aspects, the second agent is administered at a dosage which is less toxic to the subject's liver or kidney than a therapeutically effective dosage of the second agent administered as a monotherapy.

In some aspects, the second agent comprises a therapeutically effective dosage which is lower than a therapeutically effective dosage of the nanoformulated second agent administered as a monotherapy without the first agent.

In some aspects, the nanoformulated second agent is less toxic to the subject when administered as the combination therapy than as a monotherapy without the first agent.

In some aspects, the nanoformulated second agent when administered as the combination therapy is less toxic to the subject's liver and/or kidney than the same dosage of the second agent administered as a monotherapy without the first agent.

In some aspects, the method further comprises detecting the level MARCO in a sample from the subject prior to administration of the combination therapy.

In some aspects, the combination therapy is administered when the MARCO level is high in the subject.

In some aspects, the method comprises detecting the level of a liver or kidney toxicity biomarker in a sample from the subject. In some aspects, the sample is a blood sample or a urine sample.

In some aspects, the combination therapy is administered systemically. In some aspects, the combination therapy is administered to the subject by parenteral injection. In some aspects, the combination therapy is administered to the subject by intravenous, subcutaneous, or intramuscular injection.

### VII. Kits

Certain aspects of the disclosure are directed to a kit comprising a first agent and a nanoformulated second agent, wherein the first agent and the nanoformulated second agent can be combined to produce a combination therapy, wherein the first agent is capable of blocking the interaction between a Macrophage Receptor with Collagenous Structure (MARCO) and a nanoparticle, and the nanoformulated second agent comprises an anti-cancer agent or an anti-tumor agent.

In some aspects, the present disclosure provides a kit comprising a first agent, a second agent, and an excipient, wherein one or more of the first agent, the second agent, and the excipient are isolated and can be combined to produce a combination therapy, wherein the combination therapy is formulated for delivery of the first and second agent to a subject in need thereof, wherein the first agent is an agent which reduces a Macrophage Receptor With Collagenous Structure (MARCO) activity relative to a reference level of the MARCO activity, wherein the second agent is an anti-cancer agent or an anti-tumor agent, wherein at least the second agent is in the form of nanoformulation, wherein the first agent and the second agent are present in the combination therapy at concentrations which are therapeutically effective for the treatment of a tumor or a cancer upon administration of the combination therapy to the subject; and a set of instructions for producing the combination therapy and administering the combination therapy to a subject.

In some aspects, the present disclosure provides a kit comprising: a combination therapy comprising a first agent, a second agent, and an excipient, wherein the combination therapy is formulated for delivery of the first and second agent to a subject in need thereof, wherein the first agent is an agent which reduces a Macrophage Receptor With Collagenous Structure (MARCO) activity relative to a reference level of the MARCO activity, wherein the second agent is an anti-cancer agent or an anti-tumor agent, wherein at least the second agent is in the form of nanoformulation, wherein the first agent and the second agent are present in the combination therapy at concentrations which are therapeutically effective for the treatment of a tumor or a cancer upon administration of the combination therapy to the subject; and a set of instructions for administration of the combination therapy.

In some aspects, the present disclosure provides a kit comprising a first agent, a second agent, and an excipient, wherein one or more of the first agent, the second agent, and the excipient are isolated and can be combined to produce a combination therapy, wherein the combination therapy is formulated for delivery of the first and second agent to a subject in need thereof, wherein the first agent is an agent which reduces a Macrophage Receptor With Collagenous Structure (MARCO) activity relative to a reference level of the MARCO activity, wherein the second agent is an agent suitable for treatment of a disease or condition, wherein at least the second agent is in the form of nanoformulation, wherein the first agent and the second agent are present in the combination therapy at concentrations which are therapeutically effective for the treatment of the disease or condition upon administration of the combination therapy to the subject; and a set of instructions for producing the combination therapy and administering the combination therapy to a subject.

In some aspects, the present disclosure provides a kit comprising: a combination therapy comprising a first agent, a second agent, and an excipient, wherein the combination therapy is formulated for delivery of the first and second agent to a subject in need thereof, wherein the first agent is an agent which reduces a Macrophage Receptor With Collagenous Structure (MARCO) activity relative to a reference level of the MARCO activity, wherein the second agent is an agent suitable for treatment of a disease or condition, wherein at least the second agent is in the form of nanoformulation, wherein the first agent and the second agent are present in the combination therapy at concentrations which are therapeutically effective for the treatment of the disease or condition upon administration of the combination therapy to the subject; and a set of instructions for administration of the combination therapy.

Unless defined otherwise, all technical and scientific terms used herein have the same meaning as commonly understood by one of ordinary skill in the art to which this disclosure belongs. In case of conflict, the present application including the definitions will control. Unless otherwise required by context, singular terms shall include pluralities and plural terms shall include the singular. All publications, patents and other references mentioned herein are incorporated by reference in their entireties for all purposes as if each individual publication or patent application were specifically and individually indicated to be incorporated by reference.

Although methods and materials similar or equivalent to those described herein can be used in practice or testing of the present disclosure, suitable methods and materials are described below. The materials, methods and examples are illustrative only and are not intended to be limiting. Other features and advantages of the disclosure will be apparent from the detailed description and from the claims.

The following examples are illustrative and do not limit the scope of the claimed aspects.

### EXAMPLES

### Example 1. MARCO expression in Macrophages

The correlation of MARCO expression with macrophages' ability to uptake nanoparticles was tested in bone marrow-derived macrophages (BMDMs) that were MARCO negative (MARCO-Neg) or MARCO positive (MARCO-Pos).

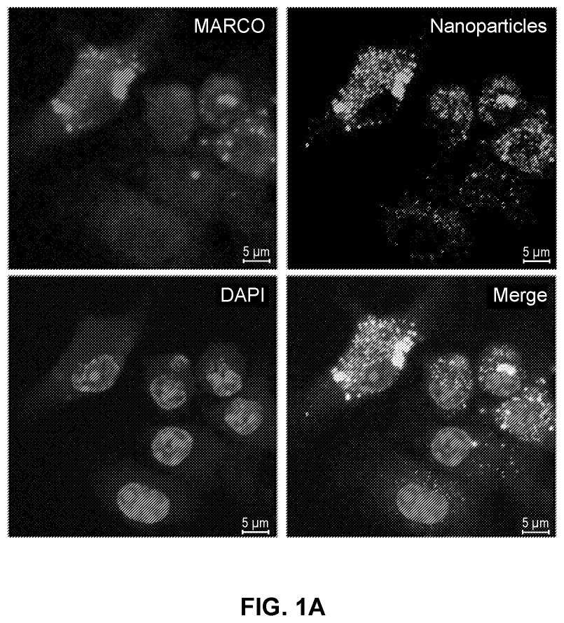

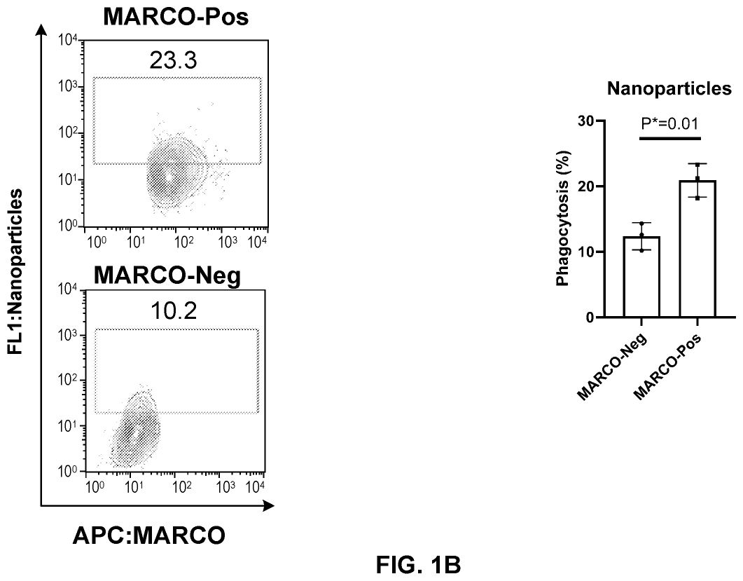

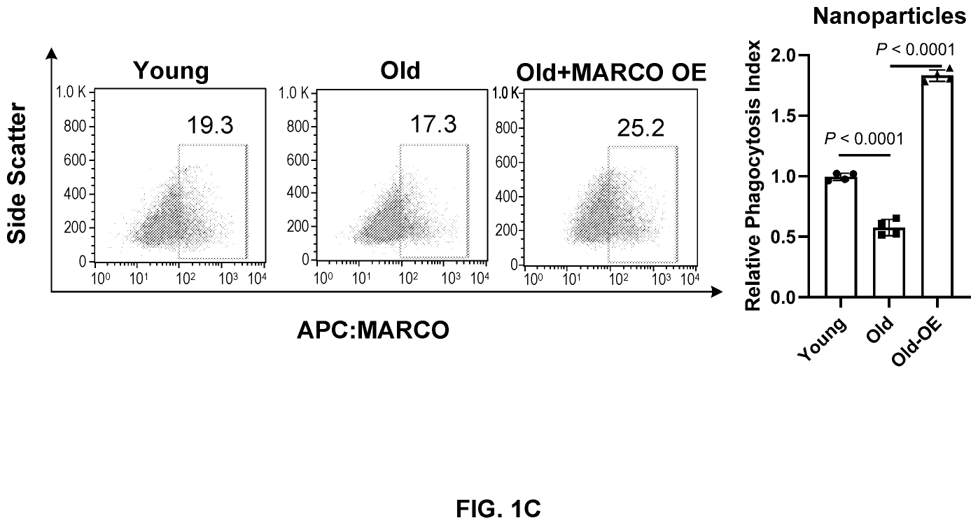

Immunofluorescence staining of bone marrow-derived macrophages (BMDMs) incubated with nanoparticles is shown in FIG. 1A. Scale bar=5 μm. The slides were blocked with blocking buffer (3% bovine serum albumin in PBS) for 1 h, followed by incubation in the dark for 1 h with primary antibodies (MARCO, human, Invitrogen #MA140315, 1:200; MARCO, mouse, R&D Systems #MAB29561/FAB29561R, 1:200; CD11b, Biolegend #101217, 1:200), at room temperature and then for 1 h with secondary antibodies (1:200, Thermo Fisher #A10042) and DAPI (4′,6-diamidine-2-phenylindole dihydrochloride, Roche, #10236276001, 1:1000). Each antibody incubation step was followed by three washes in PBS. Slides were mounted by using Vectashield mounting medium. Images were acquired with laser scanning confocal microscopes LSM 780 or LSM 880 (Carl Zeiss). Stains were quantified by ImageJ.

Nanoparticle uptake by the MARCO-positive and MARCO-negative BMDMs is shown in FIG. 1B; Left panel shows representative flow cytometry results. n=3. Unpaired t-test. Flow cytometry results showed MARCO expression on macrophages in FIG. 1C; Right panel: phagocytosis of nanoparticles. Old macrophages expressed lower levels of MARCO. Prior to flow cytometry, isolated or harvested cells were washed by PBS containing 2% FBS and fixed by 4% paraformaldehyde. All macrophages were blocked with FcR antibody (Bioxcell, Clone 2.4G2, 1 μg/mL, for mouse; BD Biosciences, Clone Fc1.3216, 1 μg/mL, for human) and with True-Stain Monocyte Blocker (BioLegend, 426101, when tandem dyes were used). Antibodies used for flow cytometry were: MARCO antibody (Human: ThermoFisher, clone PLK-1, Cat. 17-5447-42, 1:100; Mouse: R&D Systems, clone 579511, Cat. FAB2956A, 1:50). The cells were processed on a BD Accuri C6, BD FACS Calibur, Beckman Coulter Gallios, or Invitrogen Attune flow cytometer. Results were analyzed by FlowJo 10.7.2 or BD Accuri C6 v227 software.

### Example 2. Nanoparticle Uptake by Macrophages Treated with MARCO Inhibitors

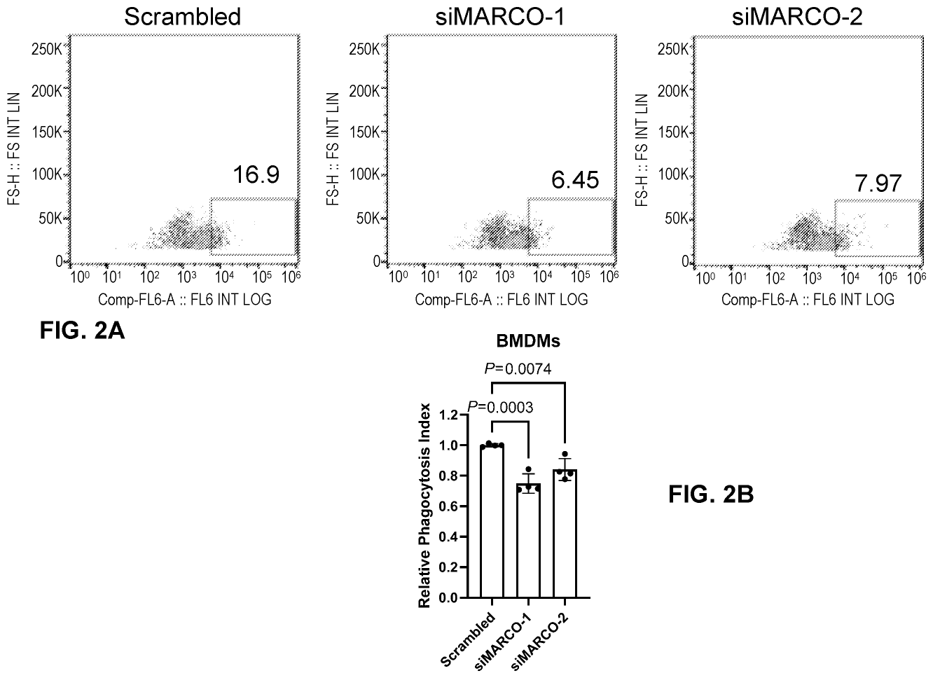

The effect of MARCO expression on nanoparticle uptake by macrophages in vitro was tested. Knockdown of MARCO decreased nanoparticle uptake by macrophages. On day 5 following macrophage collection and culture, the siRNAs were mixed with the DharmaFECT 4 transfection reagent (Horizon, T-2004-02) at a final concentration of 100 nM, after which the cells were incubated with the siRNA for 48 hours. The siRNAs used in this study were as follows: Control siRNA (Cell Signaling Technology, 6568S) and Marco siRNA (Millipore Sigma, SASI_Mm01_00200135, SASI_Mm02_00319301). Flow cytometry results of MARCO knockdown by siRNA are shown in FIG. 2A. FIG. 2B shows nanoparticle phagocytosis by macrophages after knockdown.

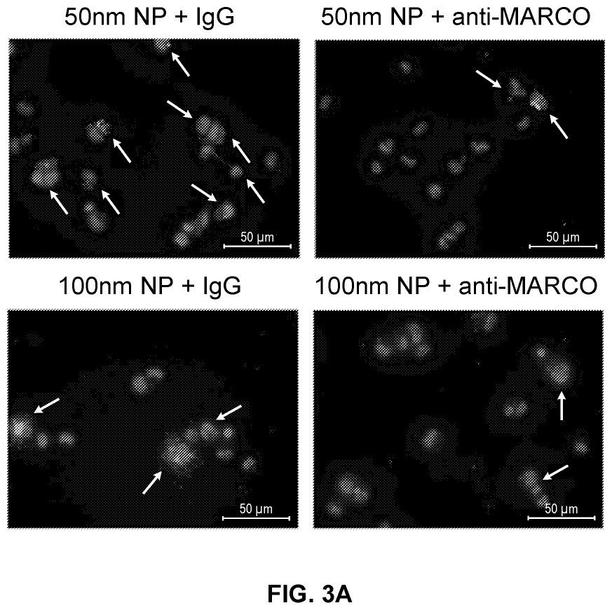

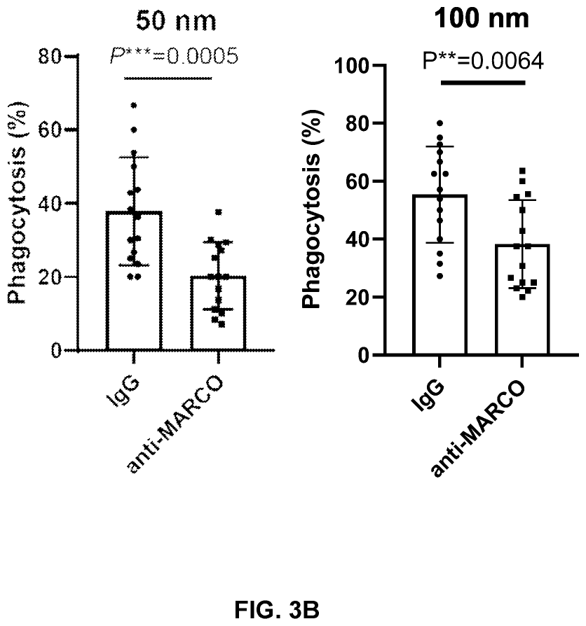

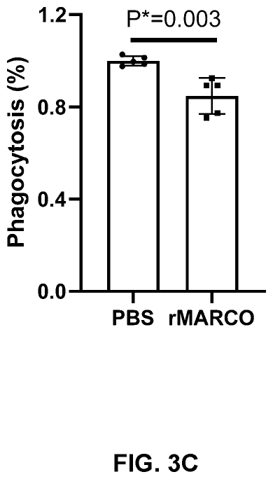

Blockade of MARCO-nanoparticle interaction decreased nanoparticle uptake by macrophages. FIG. 3A shows images of macrophages and nanoparticles incubated with or without the anti-MARCO antibody (5 μg/ml) or isotype IgG. Scale bar=50 μm. White arrows indicate cells that have taken up nanoparticles. FIG. 3B shows quantification of nanoparticle⁺ cells. Unpaired t-test. All error bars are mean±standard deviation (S.D.) FIG. 3C shows nanoparticle uptake by macrophages with or without incubation with recombinant MARCO protein.

These results showed that MARCO expression on macrophages correlates with the macrophages' ability to uptake nanoparticles. Knockdown of MARCO decreased nanoparticle uptake by macrophages. In contrast, overexpression of MARCO increased the uptake.

### Example 3. Blockade of MARCO-Nanoparticle Interaction Decreases Nanoparticle Uptake by Liver

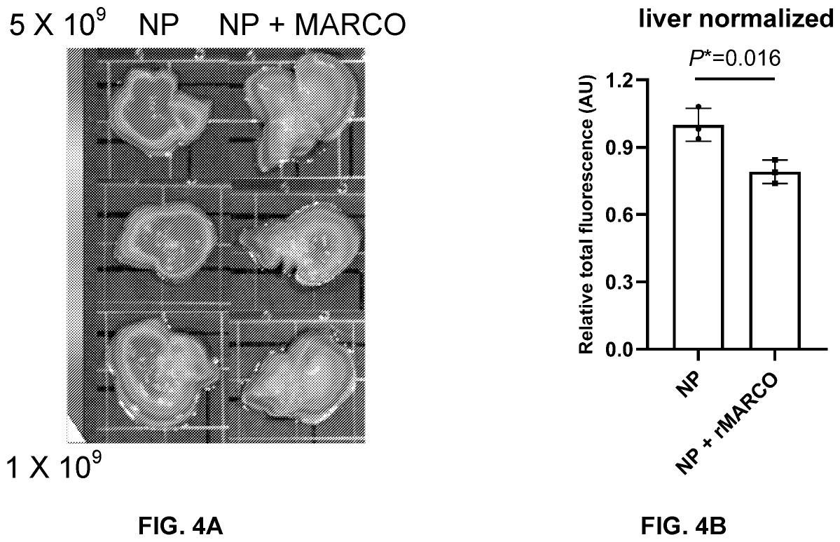

Blockade of MARCO-nanoparticle interaction decreased nanoparticle uptake by liver in mice. Two strategies for blocking the MARCO-nanoparticle interaction were tested: with antibody to directly block the MARCO receptors on the cell or with recombinant MARCO protein to saturate the MARCO binding capacity of the particles. Young female C57BL6 background mice (aged 6 weeks) were intravenously injected with fluorescence-labelled 100 nm PEGylated polystyrene nanoparticles (NP, 10\hat{ }11 particles per 20 g of body weight) with or without recombinant MARCO protein (SRCR domain, 0.3 mg/kg). Liver IVIS imaging was done 4 h after the injection. FIG. 4A shows in vivo imaging (IVIS images) of liver at 4 hours after nanoparticle administration. FIG. 4B: shows normalized total radiance (emission) of liver. Unpaired t-test. Error bars are mean±S.D. Both strategies significantly reduced the number of nanoparticles in the liver and increased the circulation time of the nanoparticles in blood (FIG. 30).

### Example 4. Blockade of MARCO Increased the Therapeutic Effect Paclitaxel-Protein Particles in Breast Cancer and Lung Cancer Models

Blockade of MARCO increased the therapeutic effect of ABRAXANE® in a breast cancer mouse model study. For this study, one million E0771 tumor cells were implanted in young female C57BL6 mice (aged 6 weeks old). Tumor growth was measured every 3 days with calipers. Tumor volume was calculated as: Volume=0.5×Length×Width². When tumor volume reached around 60-80 mm³ the treatment was started. The mice were treated with paclitaxel-protein particles (ABRAXANE®, 20 mg/kg) or ABRAXANE® plus MARCO blockade agents (anti-MARCO antibody, 2.5 mg/kg; ECD (extracellular domain), MARCO protein extracellular domain, 1.5 mg/kg; SRCR, or MARCO protein scavenger receptor cysteine-rich (SRCR) domain, 0.3 mg/kg). IgG plus 0.9% sodium chloride was used as a control. All agents were intravenously injected 3 times each week for a total of 6 treatments.

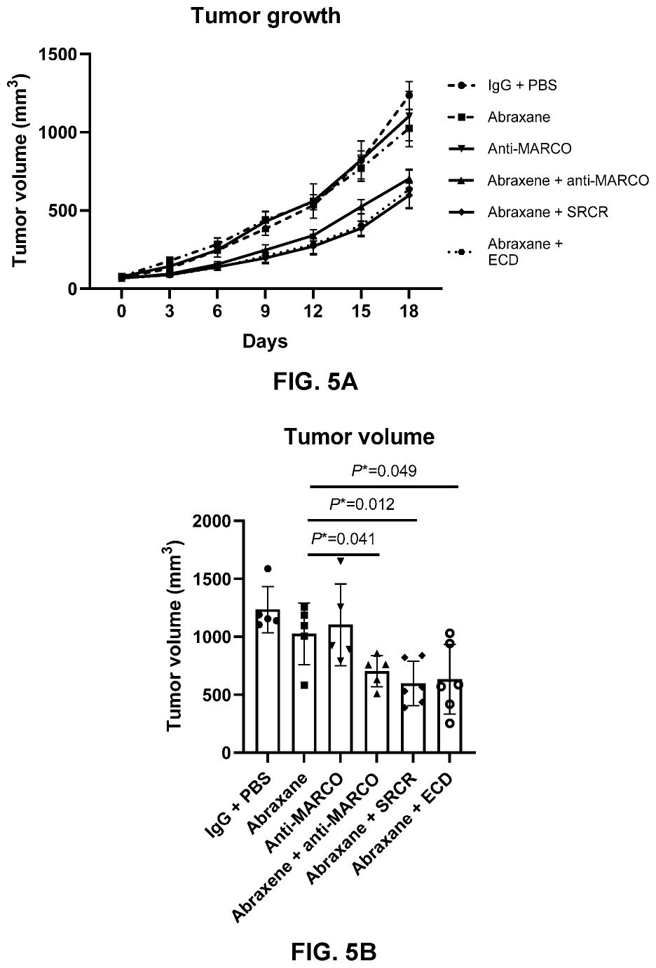

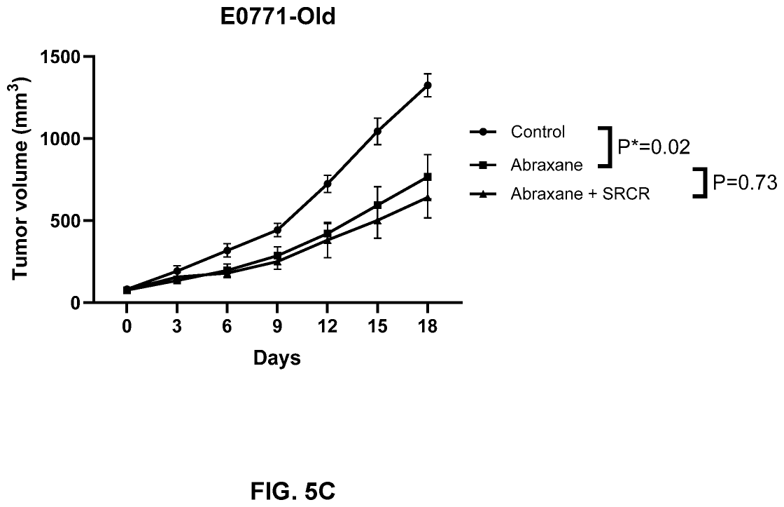

FIG. 5A shows the growth curves and FIG. 5B shows the final tumor volume in the mice treated by paclitaxel-protein particles (ABRAXANE®) or ABRAXANE® plus MARCO blockade. n=5-6 mice in each treatment arm. Un-paired t-test was used to compare tumor volumes at day 18 after ABRAXANE® vs. ABRAXANE®+MARCO blockade. Error bars are mean±S.E.M. In the E0771 model, both the ECD and the SRCR domains of MARCO enhanced the inhibition of tumor growth in young mice treated with nab-paclitaxel (ABRAXANE®) (FIGS. 5A-5B).

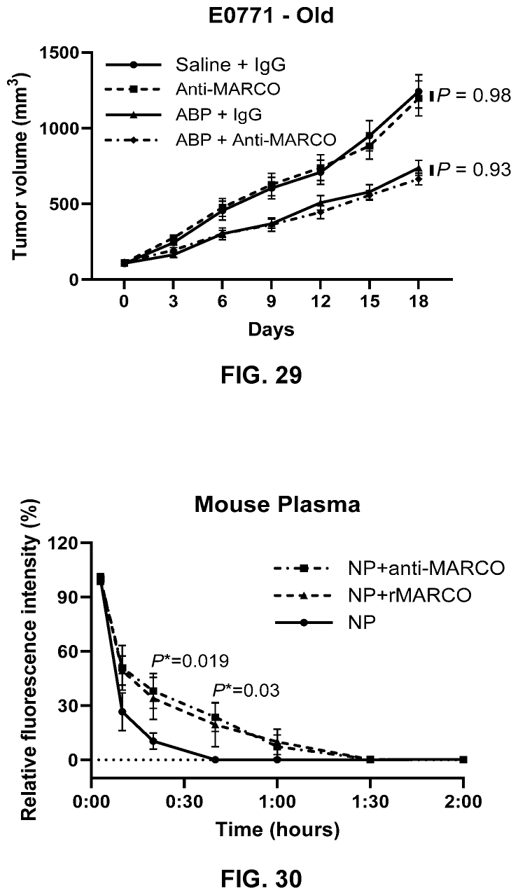

It was observed that when the same treatment experiment was carried out in the same C57BL6 mice (aged 50 weeks old) which have lower MARCO liver and blood expression, and that adding MARCO recombinant protein or anti-MARCO antibody does not enhance the treatment outcome of abraxane in older mice (FIGS. 5C and 29). One-way analysis of variance with post hoc testing was used to compare tumor volumes at day 18 after treatment. Error bars are means±S.E.M. We used the same experimental setup with the breast E0771 tumor model and treatment doses as the young animals (ABRAXANE®, 20 mg/kg, and/or MARCO protein scavenger receptor cysteine-rich (SRCR) domain, 0.3 mg/kg). This suggests that subjects having a low level of MARCO expression (e.g., older subjects) may not benefit as much from MARCO blockade.

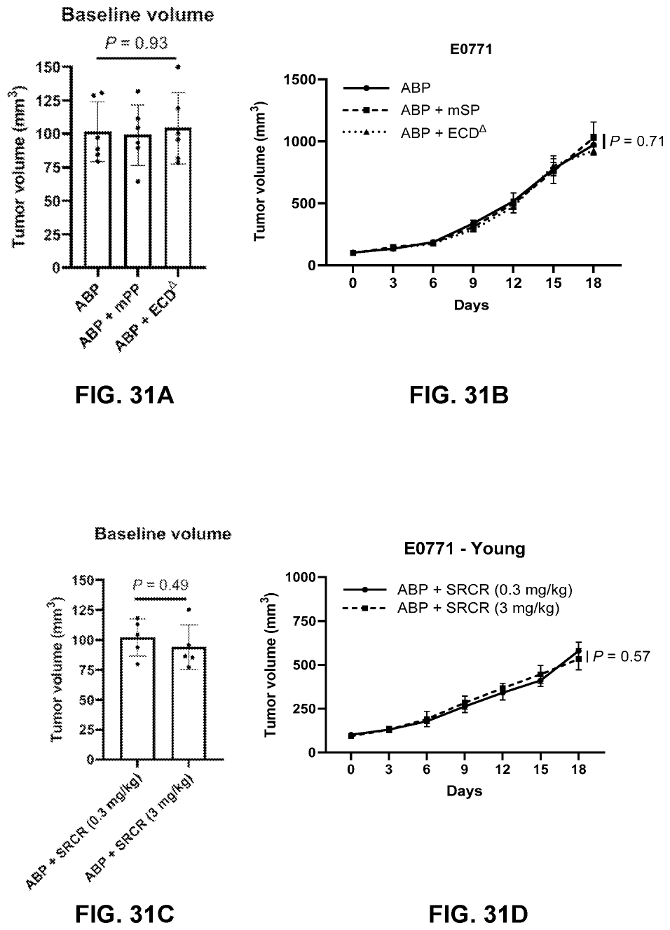

To determine the specificity of the effects of recombinant MARCO protein blockade, additional control experiments in which mouse serum protein was used as an irrelevant protein control and the ECD^{Δ} as the minimal-binding MARCO control were performed. These treatments did not improve the efficacy of nab-paclitaxel, nor did further increasing the concentration of SRCR enhance the efficacy of combination treatment (FIGS. 31A-31D).

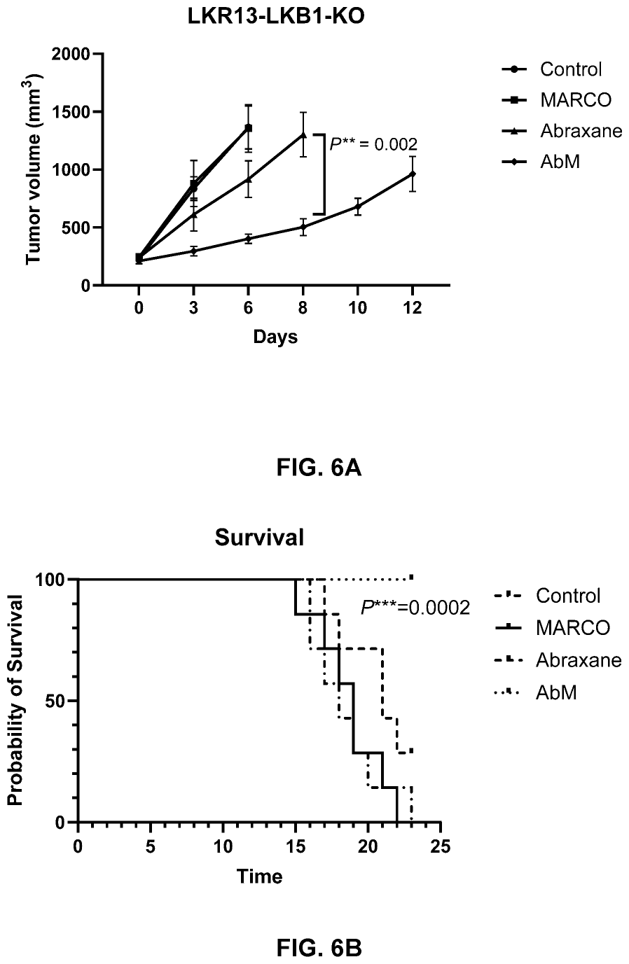

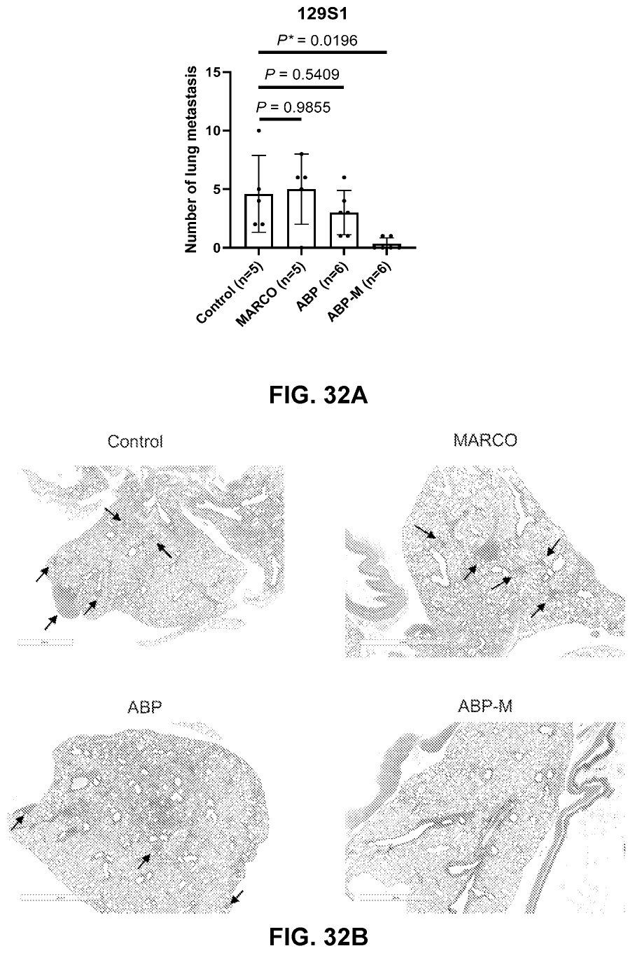

Blockade of MARCO increased the therapeutic effect of ABRAXANE® in a lung cancer mouse model study. For this study, 0.5×10⁶ LKR13-LKB1-KO lung tumor cells implanted in male 129S1 mice (aged 6 weeks old). Tumor growth was measured every 3 days with calipers. Tumor volume was calculated as: Volume=0.5×Length× Width². When tumor volume reached around 200 mm³ the treatment was started. The mice were treated with paclitaxel-protein particles (ABRAXANE®, 20 mg/kg) or ABRAXANE® plus MARCO blockade agent (MARCO (recombinant protein, SRCR domain, 0.3 mg/kg)). 0.9% sodium chloride was used as a control. All agents were intravenously injected 3 times each week for a total of 6 treatments. FIG. 6A shows growth curves in male 129S1 mice treated by paclitaxel-protein particles (ABRAXANE®) or ABRAXANE® plus MARCO blockade. n=5-6 mice in each treatment arm. Un-paired t-test was used to compare tumor volumes at day 8. Error bars are means±S.E.M. FIG. 6B shows survival curves of mice in each treatment groups. Log-rank test was used to compare the survival probability between ABRAXANE® vs. ABRAXANE®+MARCO blockade. The number of lung metastases, counted on lung tissue slides, is shown in FIG. 32A, and H&E staining images of representative lung slides in each group are shown in FIG. 32B.

These results show decreased tumor growth, decreased tumor volume and increased survival with ABRAXANE®+MARCO blockade compared to ABRAXANE® alone or MARCO blockade alone.

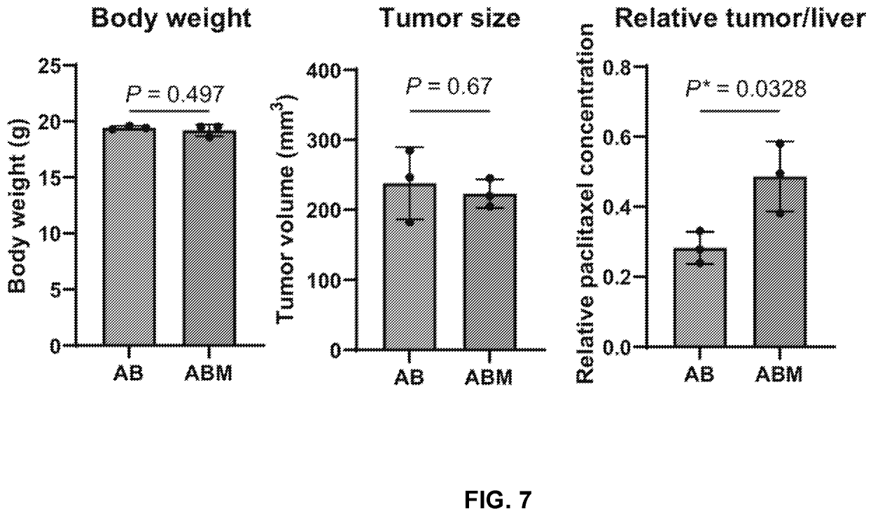

To confirm that the MARCO blockade enhanced the tumor delivery of drug, the relative paclitaxel concentrations in the liver and tumor tissues were quantified. Blockade of MARCO increased the tumor delivery of ABRAXANE®. Comparisons of body weight, tumor size, and relative tumor concentration of paclitaxel (determined by mass spectrometry) among mice injected with ABRAXANE® or ABRAXANE®+MARCO-SRCR. (FIG. 7; Un-paired t-test).

### Example 5. Blockade of MARCO Decreased Toxicity of Paclitaxel-Protein Particles In Vivo

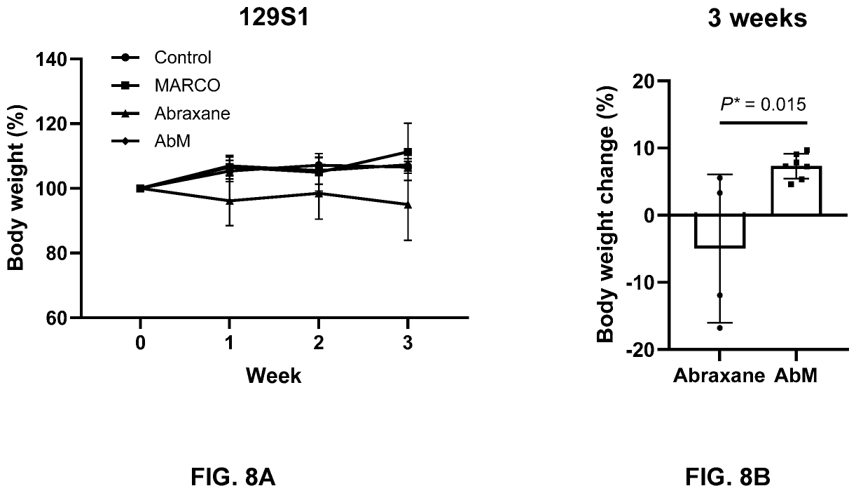

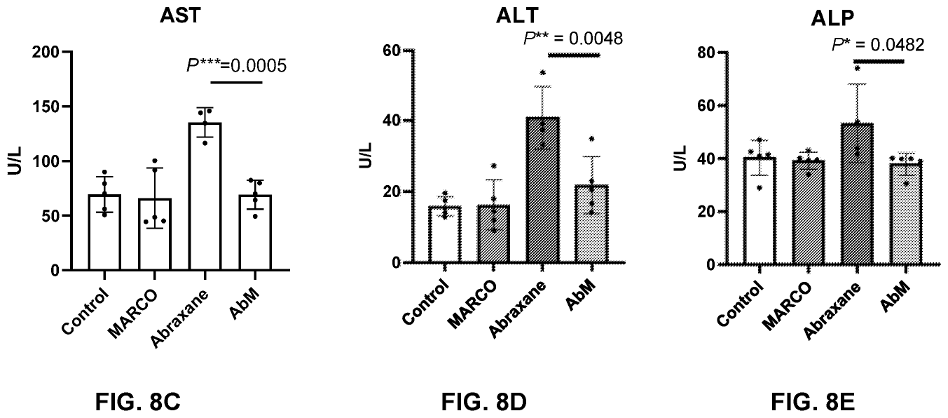

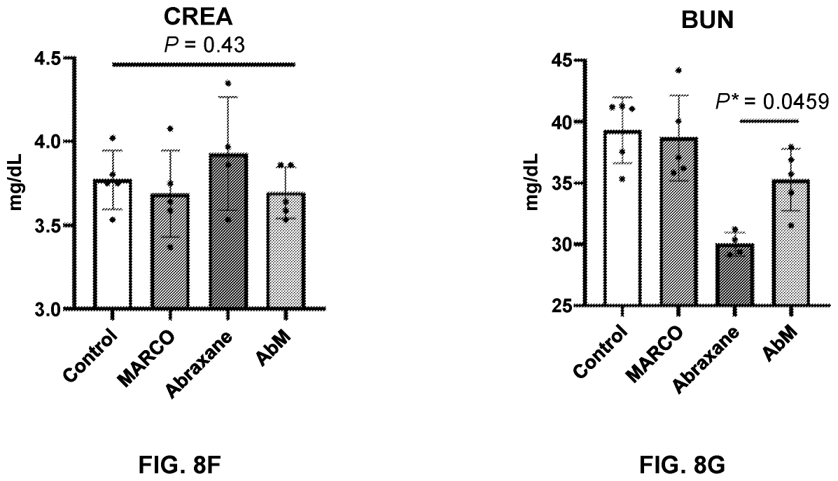

Blockade of MARCO also decreased the toxicity of ABRAXANE®. FIGS. 8A-8B: show body weight change of 129S1 mice after treatment with ABRAXANE® (20 mg/kg) or ABRAXANE®+MARCO recombinant protein (recombinant protein, SRCR domain, 0.3 mg/kg). 0.9% sodium chloride was used as a control. All agents were intravenously injected 3 times each week for a total of 6 treatments. Un-paired t-test. FIGS. 8C-8G show blood liver and kidney toxicity marker levels in different groups of treated mice compared to control. The toxicity markers included aspartate aminotransferases (AST) (FIG. 8C), alanine aminotransferases (ALT) (FIG. 8D), alkaline phosphatase (ALP) (FIG. 8E), creatinine (CREA) (FIG. 8F), and blood urea nitrogen (BUN) (FIG. 8G). One-way ANOVA with post hoc corrections for multiple comparisons was used. Analysis of blood markers of liver and kidney functions showed decreased levels of toxicity markers in the nab-paclitaxel+recombinant MARCO group, suggesting that reduced nonspecific uptake of chemotherapeutic drugs can lead to decreased systemic toxicity.

### Example 6. Blockade of MARCO Increased the Therapeutic Effect of Doxil in Breast Cancer Model

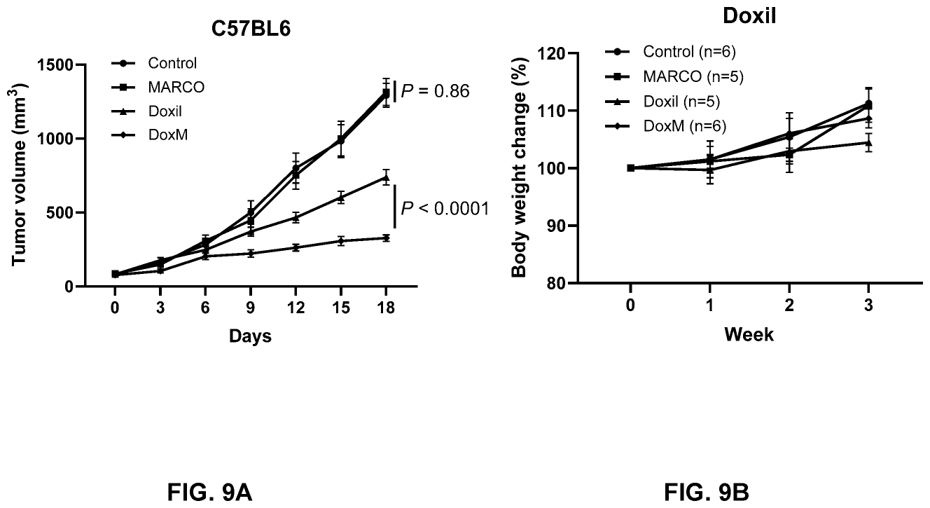

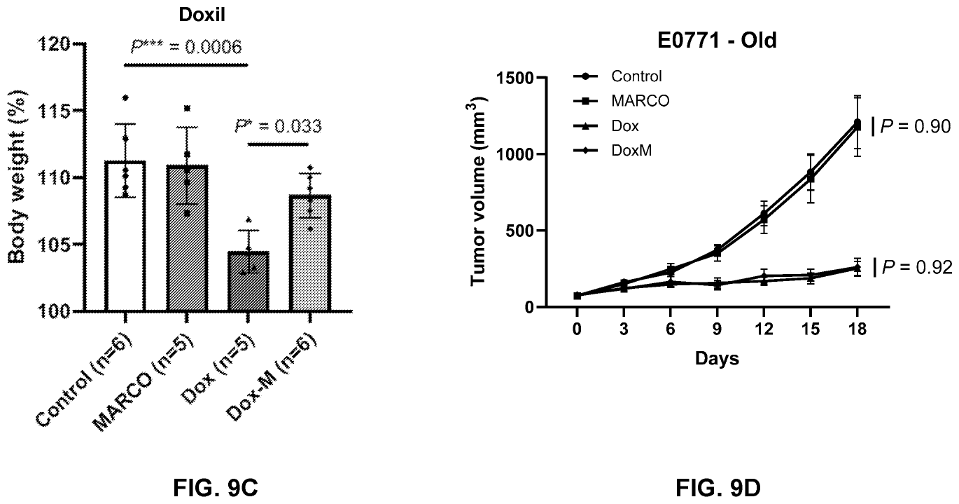

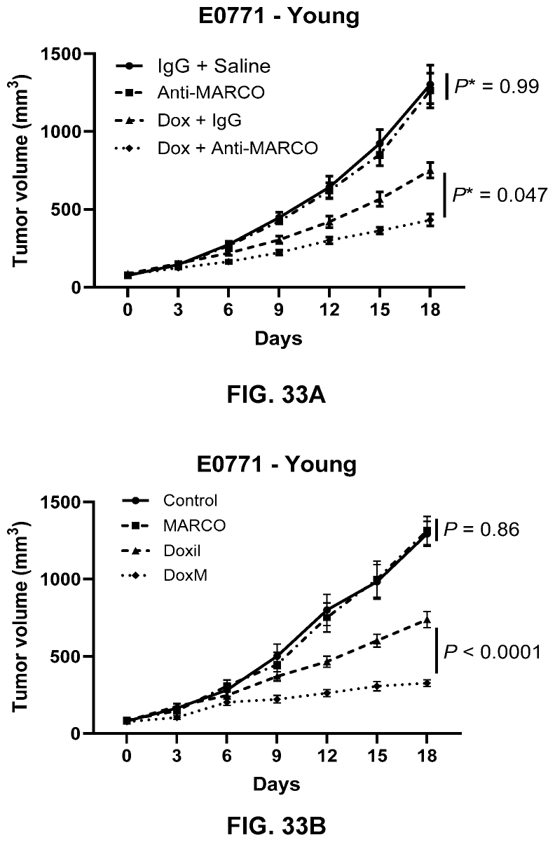

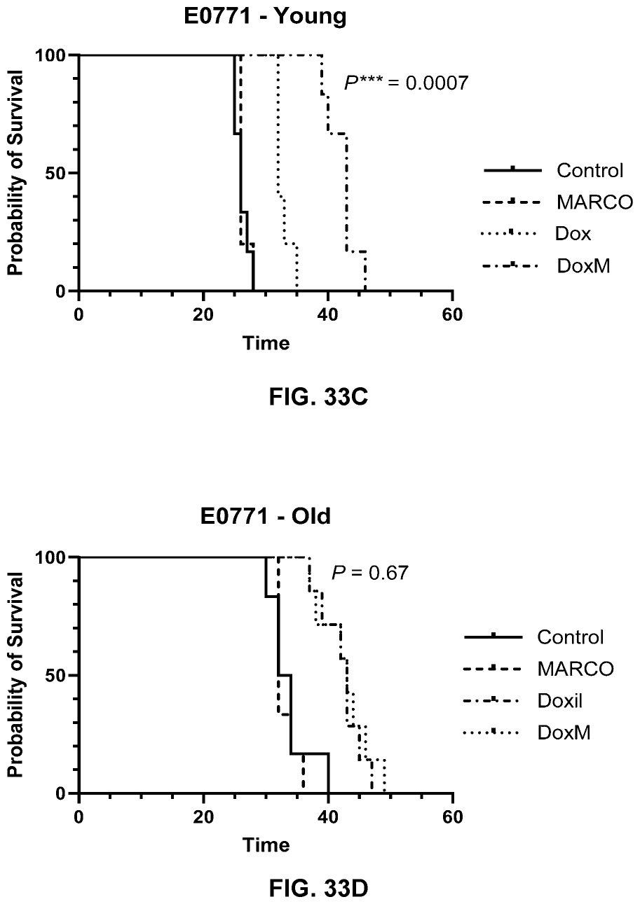

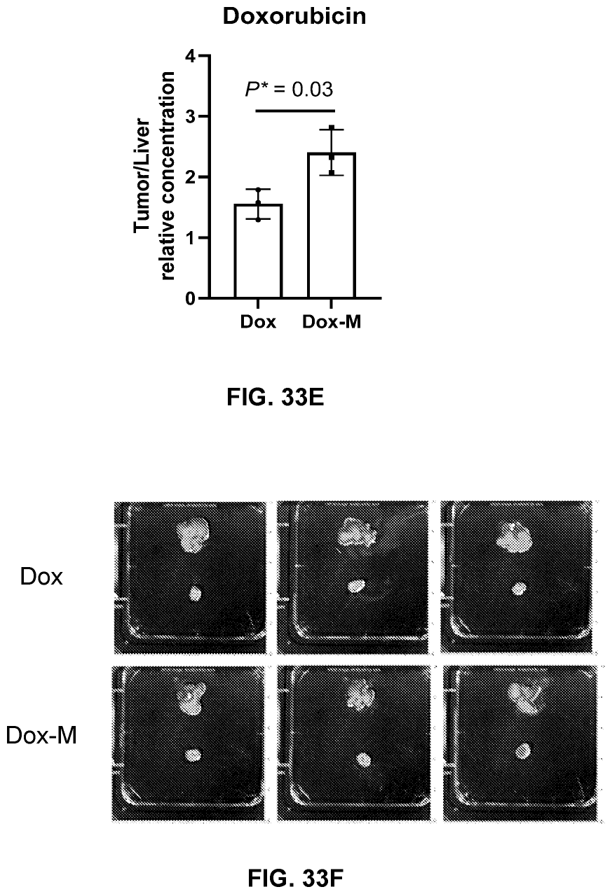

Blockade of MARCO increased the therapeutic effect of DOXIL® in a breast cancer mouse model study. For this study, one million E0771 tumor cells were implanted in young female C57BL6 mice (aged 6 weeks old). Tumor growth was measured every 3 days with calipers. Tumor volume was calculated as: Volume=0.5×Length× Width². When tumor volume reached around 60-80 mm³ the treatment was started. The mice were treated with Doxil (DOXIL®, 2 mg/kg) or DOXIL® plus MARCO blockade agent (MARCO recombinant protein, SRCR domain, 0.3 mg/kg). 0.9% sodium chloride was used as a control. All agents were injected intravenously 2 times each week for a total of 5 treatments. FIGS. 9A and 33A-33B show growth curves for the E0771 mice treated by DOXIL® or DOXIL® plus MARCO blockade. n=5-6 mice in each treatment arm. Un-paired t-test was used to compare tumor volumes at day 18. Error bars are mean±S.E.M. FIG. 9B: shows body weight change. FIG. 9C shows body weight at week 3. FIGS. 33C-33D show survival curves of young and old mice bearing E0771 tumors in the groups treated with Doxil (DOXIL®, 2 mg/kg) or DOXIL® plus MARCO blockade agent (MARCO recombinant protein, SRCR domain, 0.3 mg/kg). FIGS. 33E-33F show quantification of the relative Doxil fluorescence intensity per mg of tissue, and IVIS images of liver and tumor in mice treated with Doxil (DOXIL®, 2 mg/kg) or DOXIL® plus MARCO blockade agent (MARCO recombinant protein, SRCR domain, 0.3 mg/kg), further indicating that addition of recombinant MARCO protein increased the relative doxorubicin concentrations in tumors.

It was also observed that when the same treatment experiment was carried out in the same C57BL6 mice (aged 50 weeks old) which have lower MARCO liver and blood expression, and that adding MARCO recombinant protein does not enhance the treatment outcome of Doxil in older mice (FIG. 9D). Control vs. MARCO or Doxil vs. DoxM were compared with unpaired/tests. Error bars are means±S.E.M. The present inventors used the same experimental setup with the breast E0771 tumor model and treatment doses as the young animals (DOXIL®, 2 mg/kg, and/or MARCO protein scavenger receptor cysteine-rich (SRCR) domain, 0.3 mg/kg). This suggests that subjects having a low level of MARCO expression (e.g., older subjects) may not benefit as much from MARCO blockade.

### Example 7. Biological Aging Affects Nanomedicine Clearance and Effectiveness

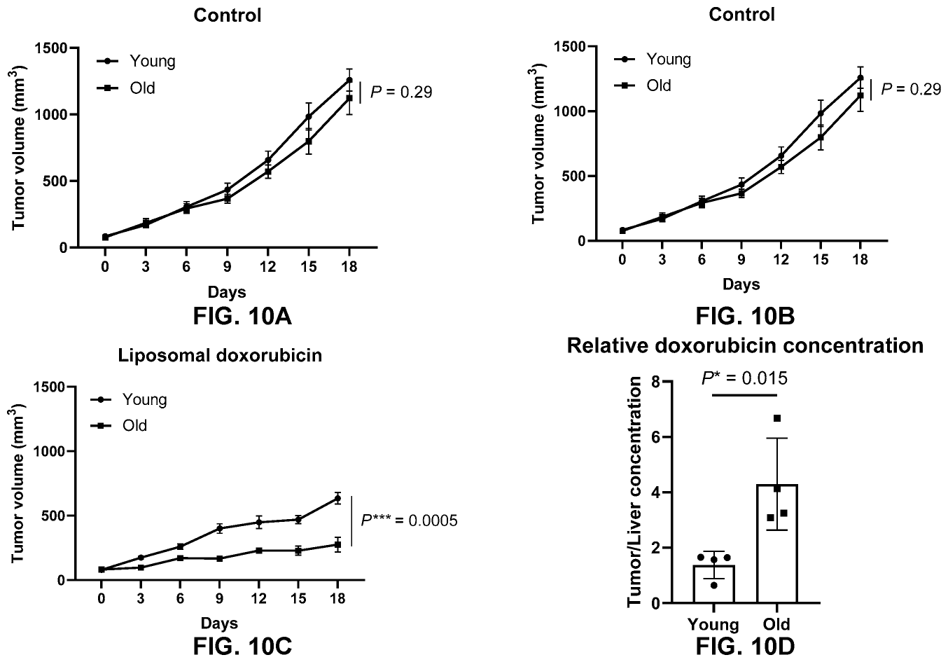

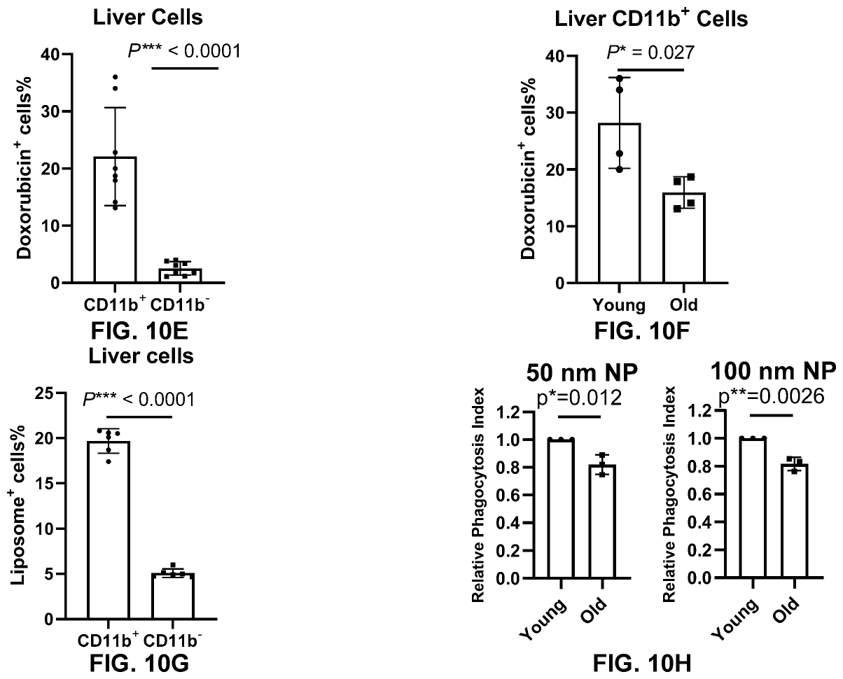

To test if the clearance and effectiveness of cancer nanomedicines is affected by aging, treatment efficacy in animals of different ages was tested. The E0771 breast tumor was implanted into young and old mice. The tumor growth in the saline control group was comparable between young and old mice (FIG. 10A). However, treatment with the same doses of nanoparticle albumin-bound paclitaxel or liposomal doxorubicin led to better tumor control in the old animals (FIGS. 10B-10C). To help understand the difference in treatment efficacy between young and old animals, it was examined if any disparities were present in the tumor vasculature, because blood vessels have a vital role in tumor drug delivery (Chen, Y. et al. 2019. Adv Sci (Weinh) 6, 1802070). To stain tumor blood vessels, tumors were fixed with 4% paraformaldehyde for 2 h followed by sucrose incubation (15% sucrose for 24 h and then 30% sucrose for 48 h) at 4° C. The tissues were next embedded in OCT at −80° C., sectioned into 20-μm thick frozen sections, and stained with the endothelial marker CD31 (BD Bioscience, Cat. #550274, 1:100) and the pericyte marker NG2 (Chemicon, Cat. #MAB5320, 1:1000) followed by secondary antibodies (1:200, Thermo Fisher #A10037, A21206). The samples were also stained with DAPI. The intensity of CD31 and NG2 was quantified by ImageJ.

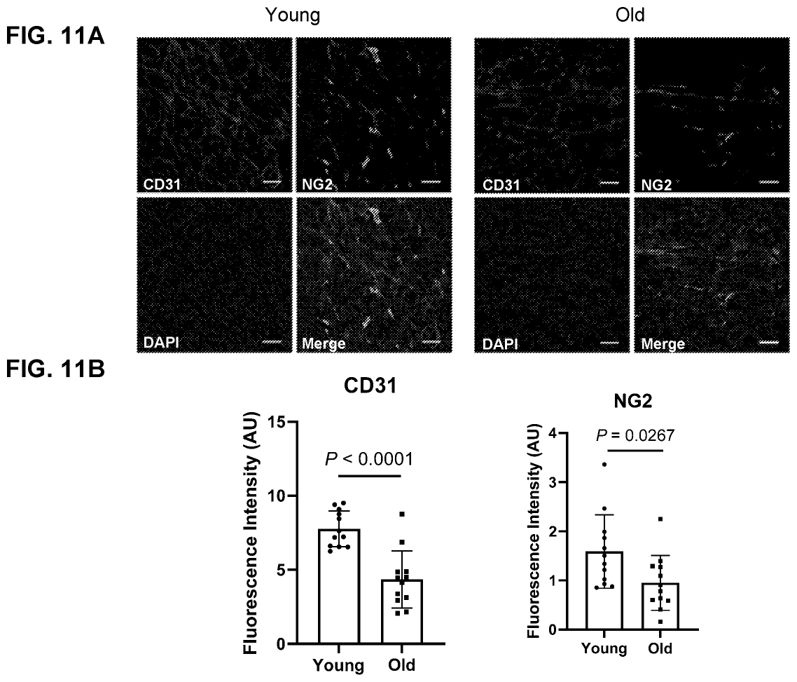

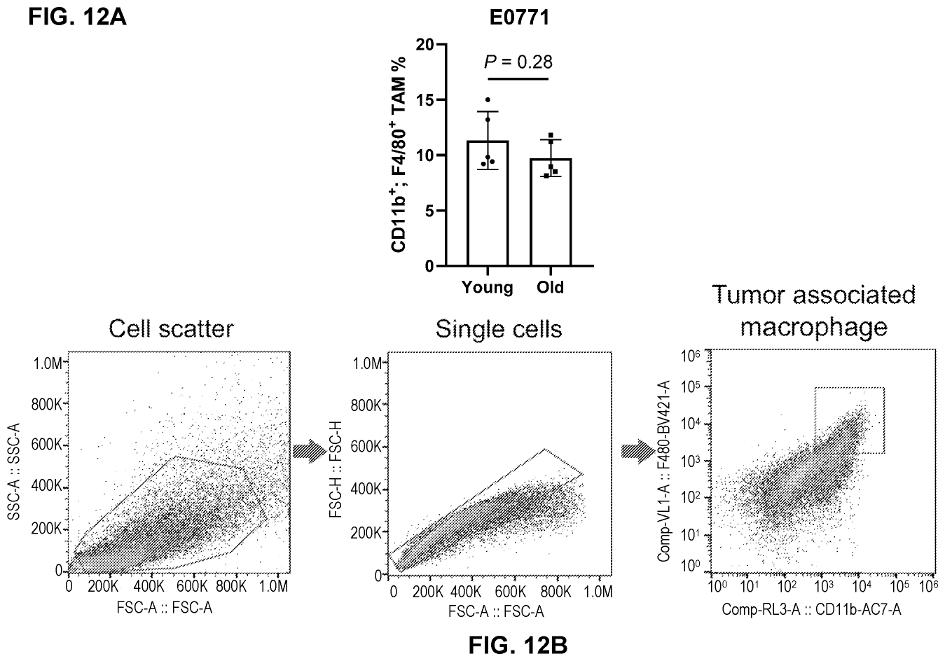

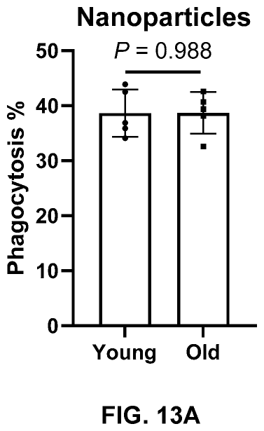

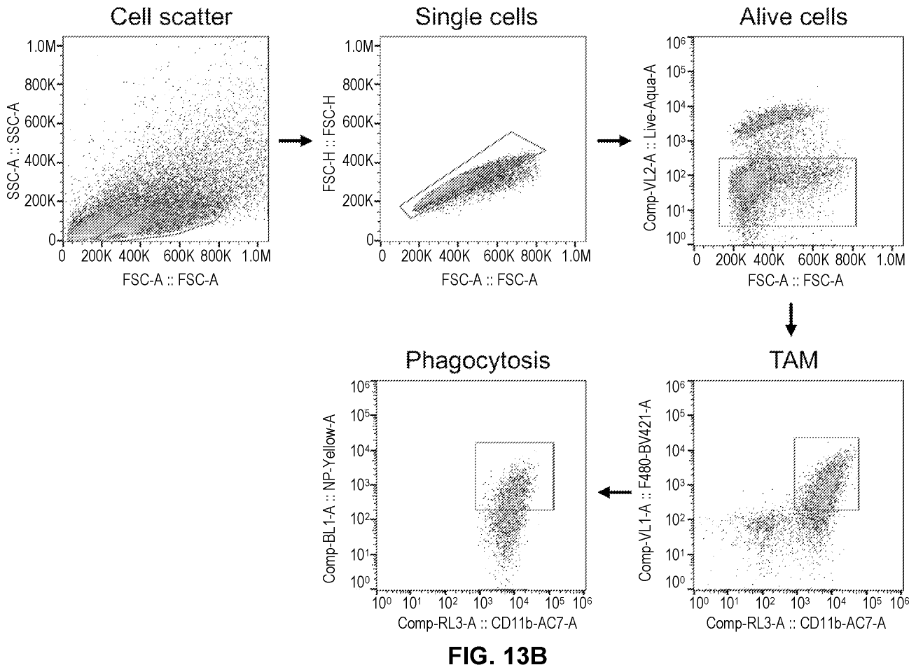

Consistent with previous reports (Pili, R. et al. 1994. J Natl Cancer Inst 86, 1303-1314), it was found that fewer blood vessels formed in tumors from the old mice (FIGS. 11A-11B). The phagocytosis ability of tumor-associated macrophages (TAMs) also had no significant differences (FIGS. 12A-12B and 13A-13B). Next, ex vivo fluorescence imaging of doxorubicin in mouse organs was performed and it was found that most of the doxorubicin was taken up by the liver (data not shown). Comparisons of drug concentrations revealed that more drugs had been delivered to the tumor in the young mice relative to the liver of the old mice (FIG. 10D).

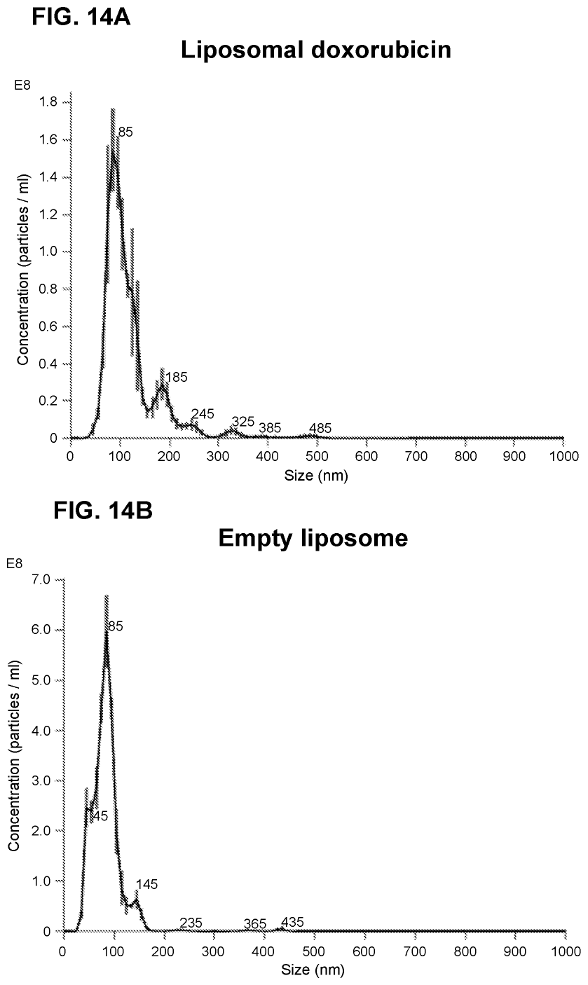

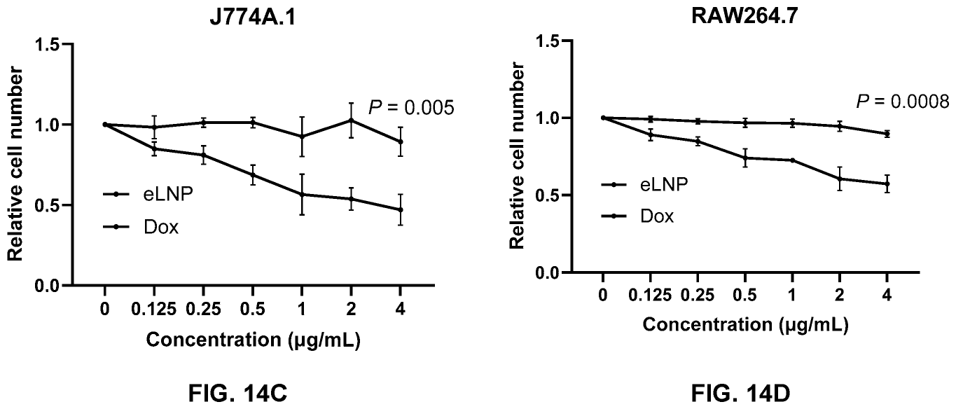

Clearance of nanoparticles by the liver is done predominantly by macrophages (Ouyang, B. et al. 2022. Mol Pharm 19, 6, 1917-1925). Indeed, large amounts of doxorubicin were found in digested liver cells expressing CD11b, a marker of monocytes and macrophages (FIG. 10E). Moreover, the CD11b⁺ cells in livers from young mice took up more doxorubicin than did the older mice (FIG. 10F). To assess the possibility that doxorubicin itself is cytotoxic, an empty fluorescent liposome of the same formulation and size as the liposomal doxorubicin was synthesized (FIGS. 14A-14B). NBD-labeled liposomes were prepared by solvent injection as follows. Briefly, HSPC/cholesterol/PEG-DSPE (mass ratio=3/1/1; Avanti Polar, #840058P and #880120P) was dissolved in ethanol at a concentration of 10 mg/mL. NBD-PE (Avanti Polar, #810156P) was then dissolved in phospholipid solution (1 molar % of total lipids). The lipid phase was injected into PBS (v/v=1/10) at 37° C. under vortex mixing. The obtained liposome solution was then dialyzed under slow agitation for 5 h to remove the ethanol, the liposome solution was concentrated by using a centrifugal filter (10-kDa cutoff), and the particle concentration in the liposome solution was measured by Nanosight. Similar results with the empty liposomes were observed (FIG. 10G). To determine if this finding could extend to other nanomaterials, including FITC-labeled nab-paclitaxel and PEGylated nanoparticles, phagocytosis assays of CD11b⁺ cells isolated from livers were performed.

Nab-paclitaxel was labeled with fluorescein isothiocyanate (FITC) with a FluoReporter FITC Protein Labeling Kit (Thermo Fisher, F6434) according to the manufacturer's instructions. The concentration of final purified labeled Abraxane was determined with a Pierce BCA Protein Assay Kit (Thermo Fisher, 23225). FITC emission was verified with a Magellan spectrometer.

For 50-nm and 100-nm nanoparticle PEGylation, carboxylate-modified polystyrene 50 nm nanoparticles (Magsphere Inc., yellow-green fluorescent) and 100 nm nanoparticles (ThermoFisher, F8803: yellow-green fluorescent; F8799: infrared) were mixed with 2% amine-PEG (ThermoFisher, 046924MD) and a 1:10 molar ratio of 1-ethyl-3-(3-dimethylaminopropyl) carbodiimide hydrochloride (EDC) (ThermoFisher, 77149) and sulfo-N-hydroxysulfosuccinimide (NHS) (ThermoFisher, 24510) with stirring at room temperature for 60 min. The nanoparticles were then washed with PBS by centrifuging through Amicon Ultra-15 Centrifugal Filter Units (Millipore Sigma, UFC905008) to remove unreacted chemicals. The final concentrated nanoparticles were resuspended in sterile PBS, and the concentration and diameter were determined by Nanosight (Malvern Panalytical, NS300) and analyzed by Nanoparticle Tracking Analysis (NTA) software 3.40.

For the nanoparticle phagocytosis assay, cells were plated in Costar 24-well Clear Flat Bottom Ultra-Low Attachment Multiple Well Plates (Corning, 3473) at a density of 10⁵ cells/well. Nanoparticles or drug were added to the wells at the following concentrations: nanoparticles, 10²-10⁴/cell; Abraxane, 1 μg/mL; doxorubicin (Doxil), 20 μg/mL. Plates were then transferred to a cell culture incubator, and 2 hours later the cells were collected and washed followed by fixation with 4% paraformaldehyde. The cells were stained with fluorescence-labeled antibodies and analyzed by flow cytometry. The mean fluorescence intensity or the percentage of fluorescence-positive cells was used to compare the uptake of nanoparticles or drug.

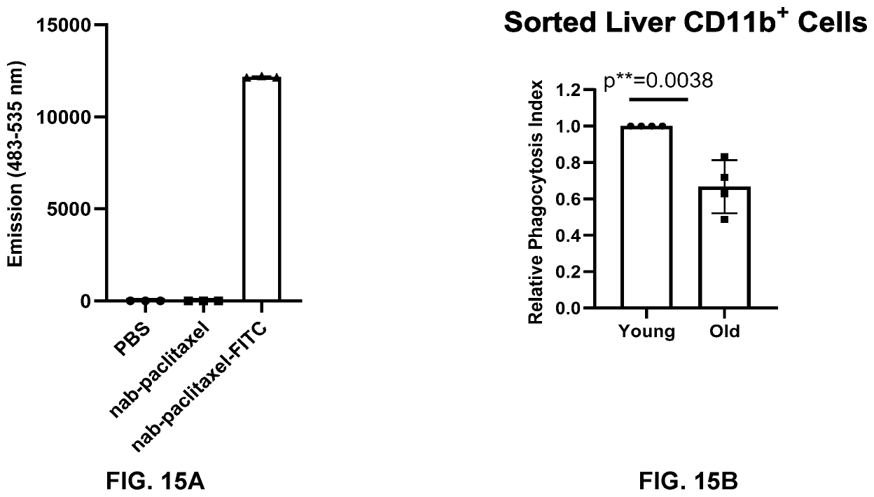

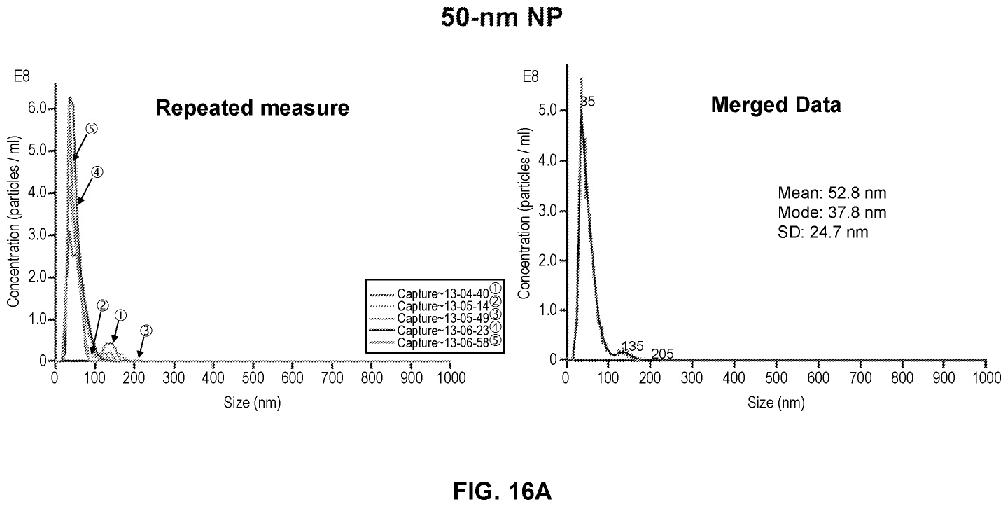

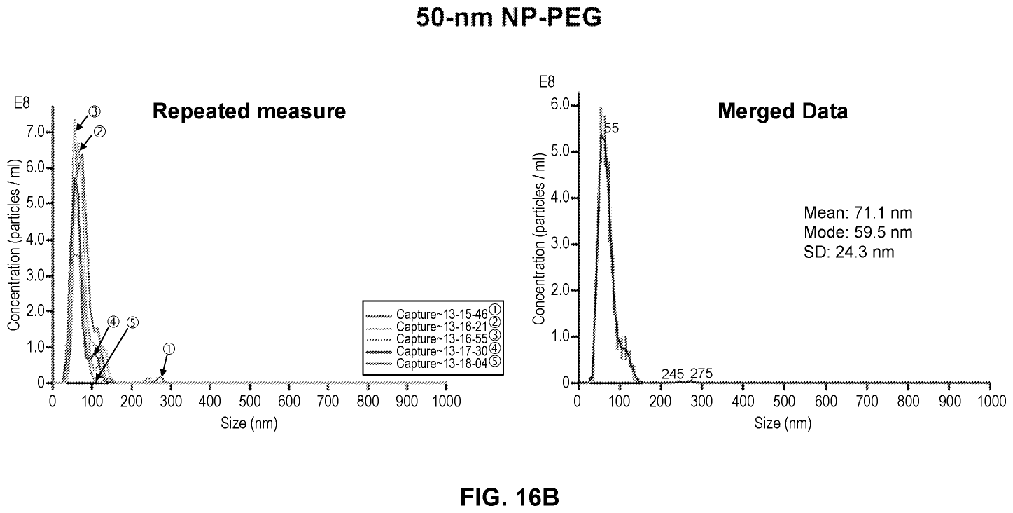

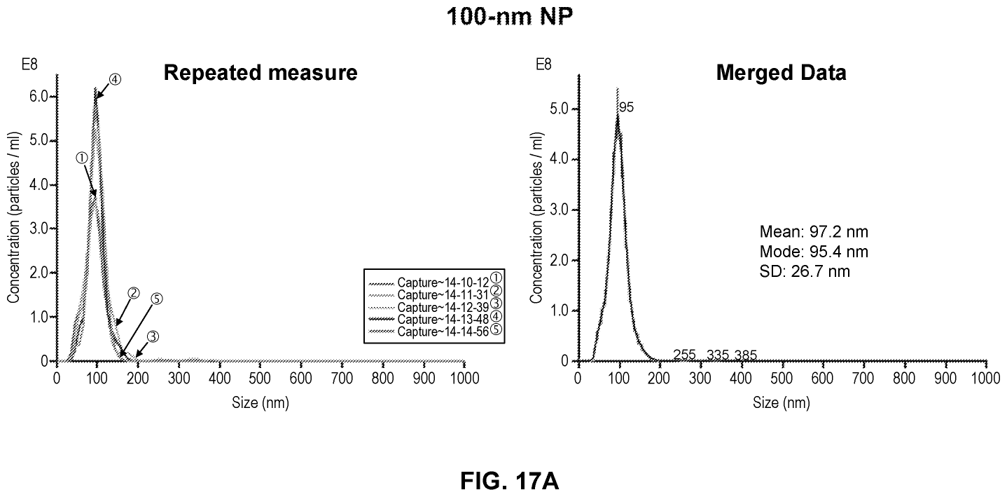

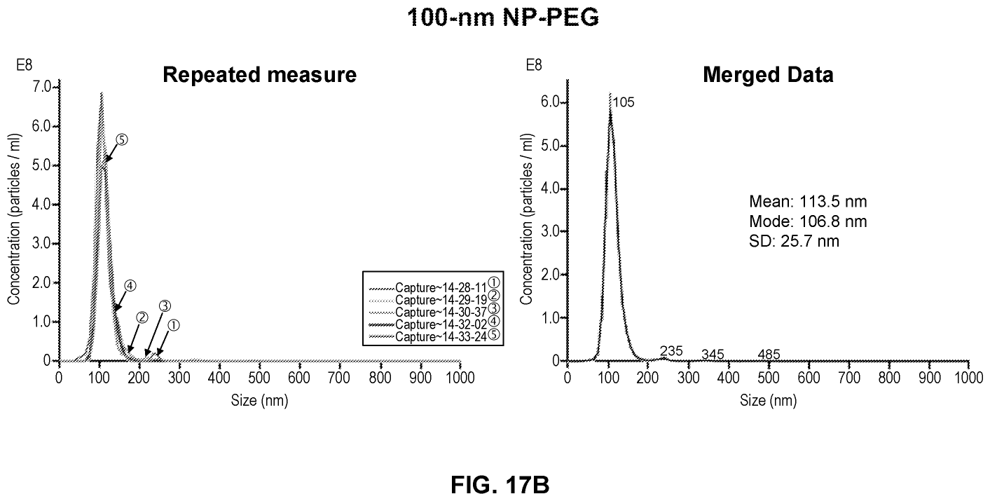

It was found that the liver CD11b⁺ cells from the old mice had lower uptake of nab-paclitaxel and PEGylated polystyrene nanoparticles (FIG. 10H and FIGS. 15-17). These findings suggest that the ability of liver CD11b⁺ cells to take up several types of nanoparticles diminishes with age.

### Example 8. Differential Expression of Phagocytosis Genes in Aged and Young Liver Macrophages

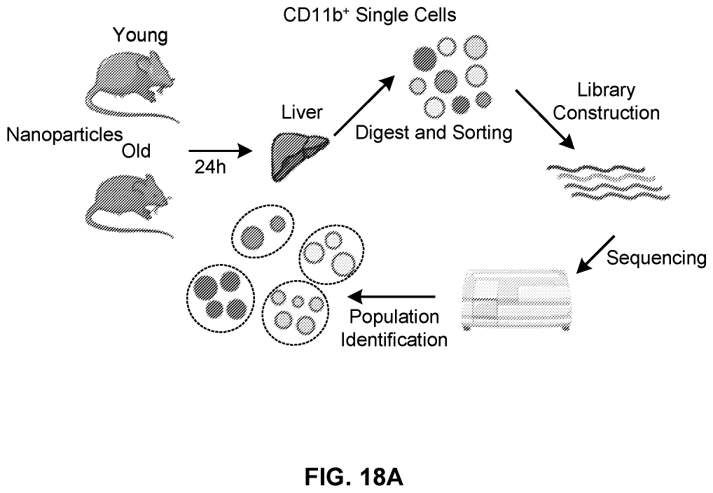

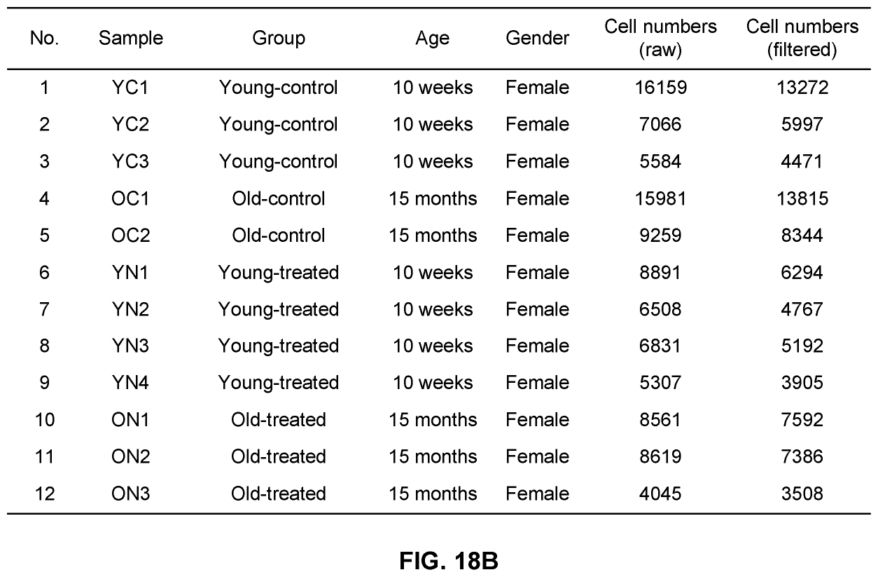

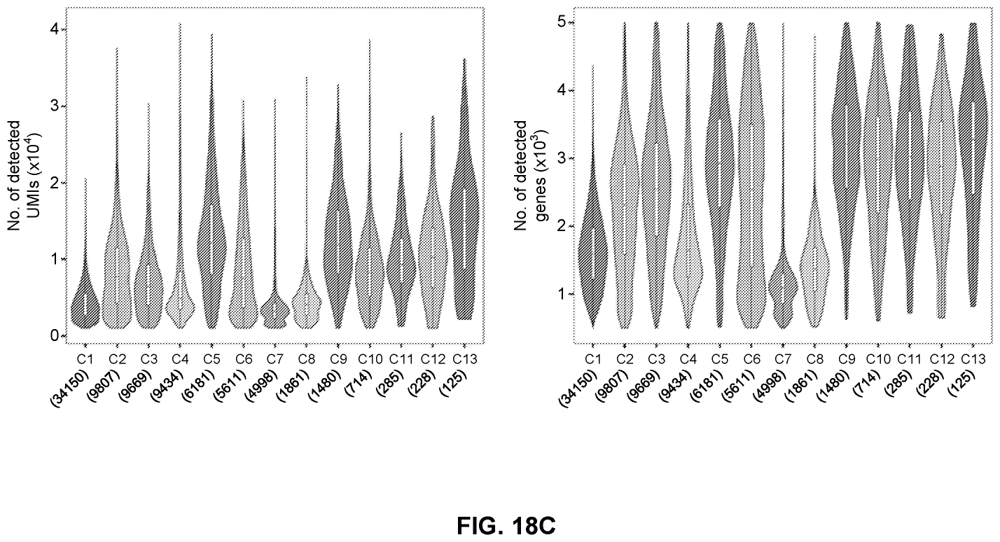

To understand transcriptomic differences between CD11b cells in livers of old and young mice and their responses to nanoparticle exposure, single-cell RNA-sequencing was performed (FIG. 18A). Single-cell 3′ gene expression libraries for each of the samples were prepared separately by following the protocols for the 10× Genomics Chromium Single-Cell 3′ Library & Gel Bead kit V3. Cells and gel beads containing the poly-T primer sequence connected with cell barcode and unique molecular identifier were wrapped by “oil droplets” to form Gel Bead in Emulsion, in which cells were lysed and completed reverse transcription according to the manufacturer's instructions. RNA transcripts from single cells were uniquely barcoded. After reverse transcription, barcoded cDNAs were purified, amplified, end-repaired, and ligated with Illumina adapters to generate a single multiplexed library. All libraries were sequenced on an Illumina Novaseq 6000 platform.

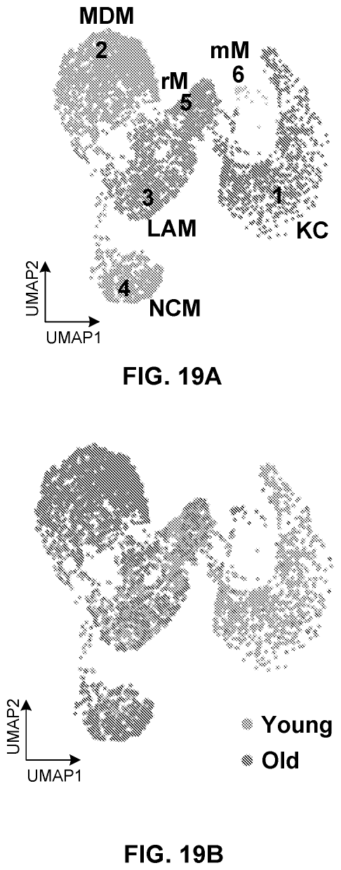

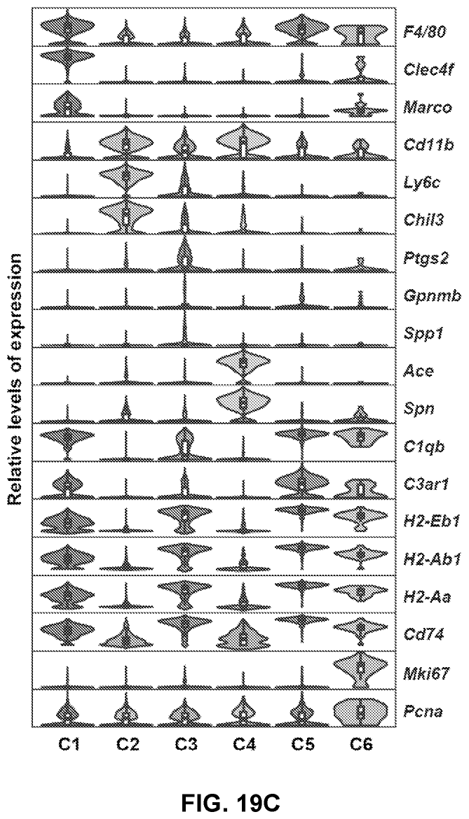

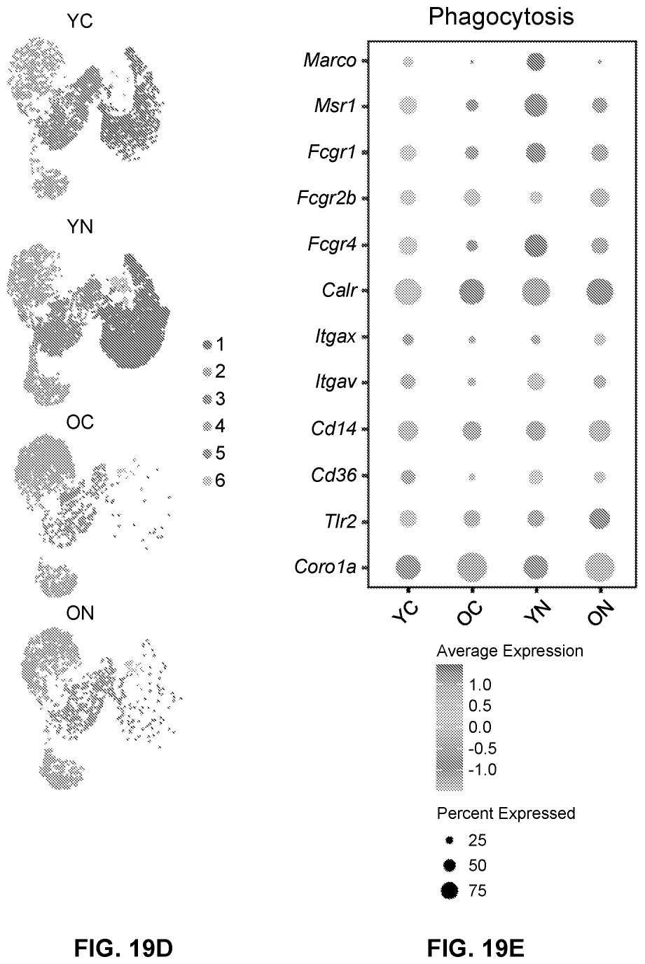

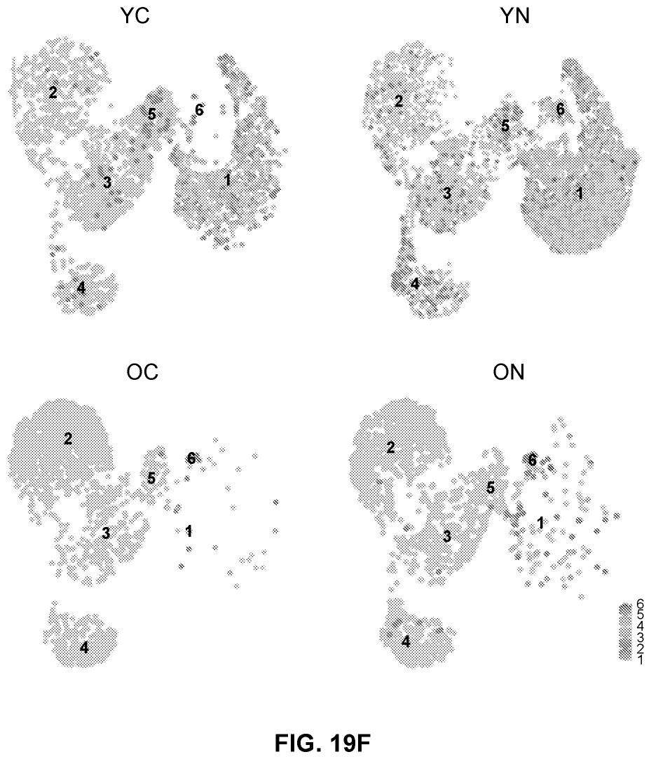

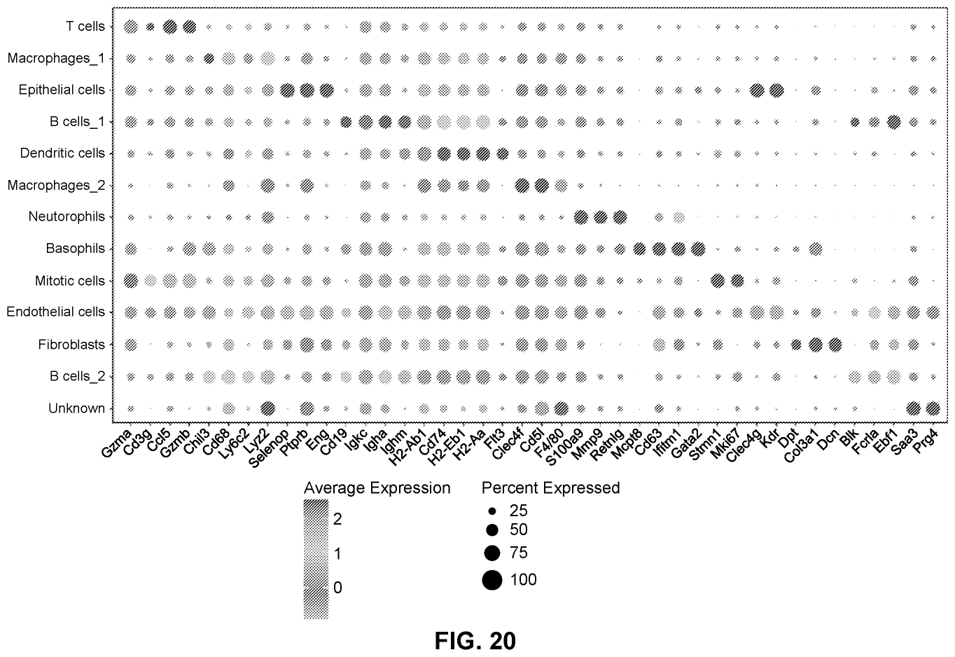

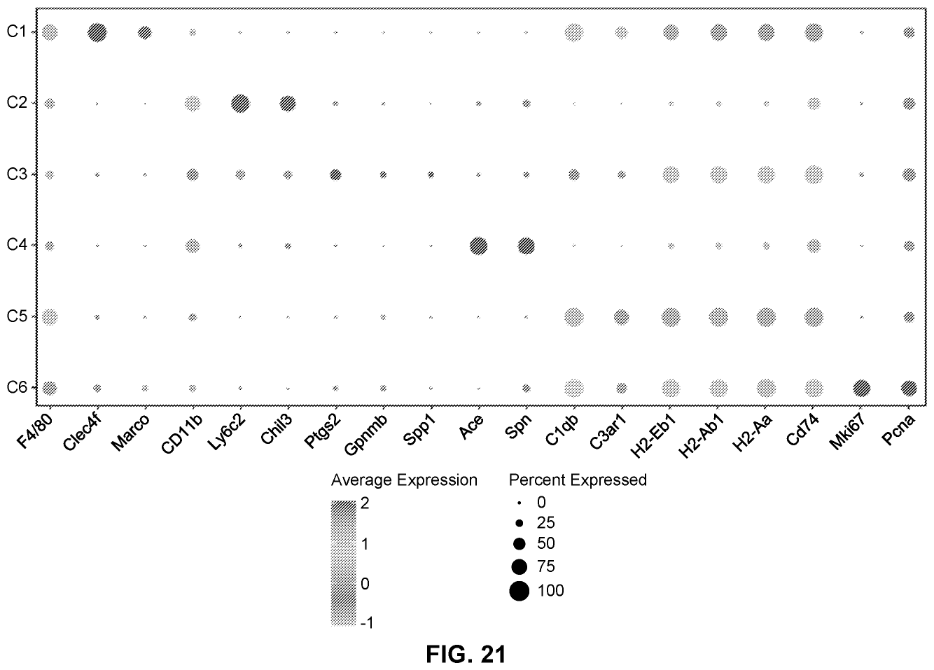

CD11b⁺ liver macrophages from young and old control mice were compared. After removing the non-phagocytic cells through the expression distribution of classical markers, the remaining cells were presented in a separate UMAP (uniform manifold approximation and projection) to further characterize macrophage subpopulations and were found to form 6 clusters (FIGS. 18B-18C, 19A-19B, and 20-21). Two main classes of macrophages were detected-tissue-resident macrophages (F4/80, Clec4f⁺, CD11b^{low}: clusters 1, 5, and 6); and monocyte-derived macrophages (Ly6c⁺, Chil3⁺, CD11b^{high}: clusters 2, 3, and 4) (FIG. 19C). The dominance of cell populations was different in the young and old livers, with old livers having fewer tissue-resident macrophages and more monocyte-derived macrophages (FIGS. 19B-19C).

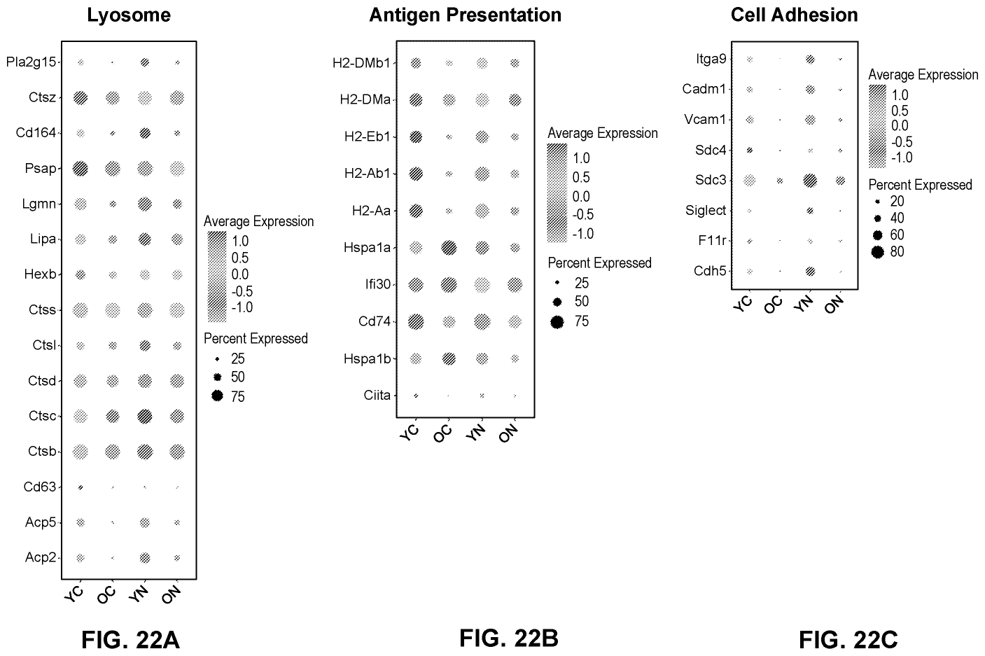

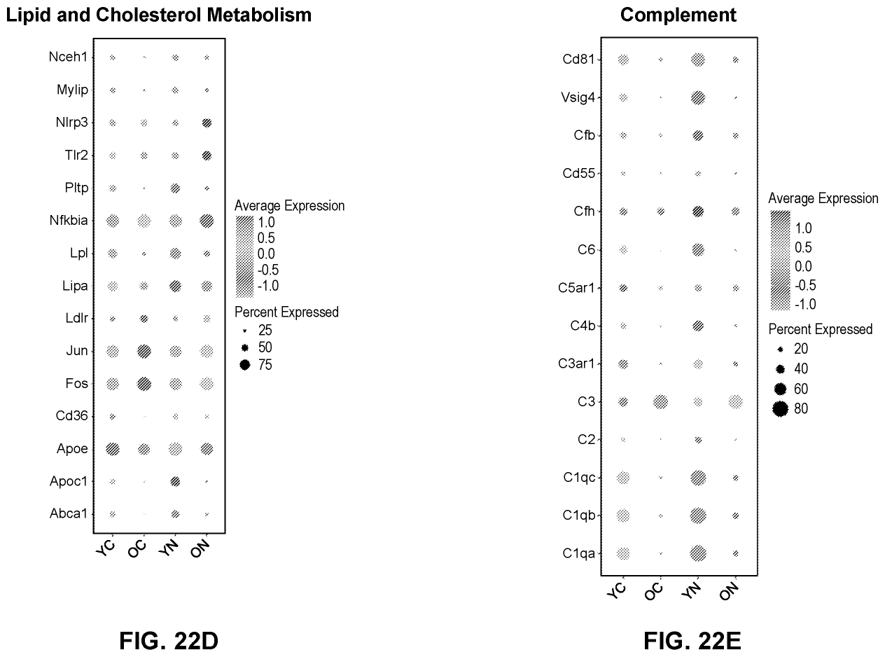

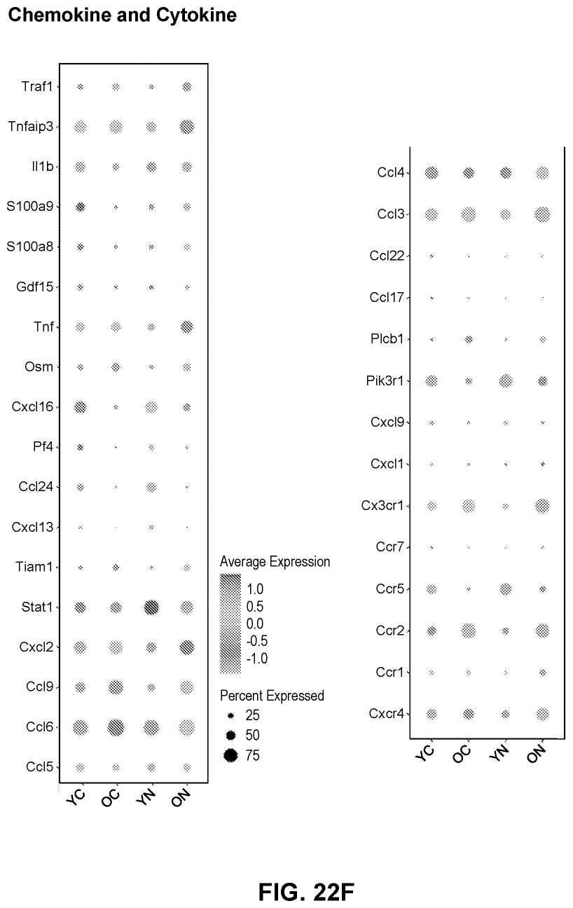

Differences in how the nanoparticles interact with liver macrophages were then analyzed. In the young liver, the number of cells in cluster 1 increased substantially after nanoparticle injection, making cluster 1 the predominant population (FIG. 2d). In the old liver, although the number of cells in cluster 1 also increased, the final number was still far fewer than in the young (FIG. 19D). Gene set variation analysis (GSVA) further revealed that young macrophages had elevated activity in phagosome, lysosome, and antigen presentation pathways (data not shown). The expression of individual genes in phagocytosis-related pathways was analyzed and it was found that young liver macrophages had higher expression of most genes and were overall more responsive to stimulation by nanoparticles (FIG. 19E). Further analyses of individual genes involved in nano-bio interactions revealed expression-level disparities in several pathways (FIGS. 22A-22F).

Because cluster 1 was the most responsive to nanoparticle stimulation and because it also differed the most between young and old, whether a therapeutic target on cluster 1 cells was present that might decrease nanoparticle uptake in the young livers was investigated. Among the liver macrophages, Marco expression was detected mainly in clusters 1 and 6 and was significantly decreased in the old macrophages (FIG. 19F).

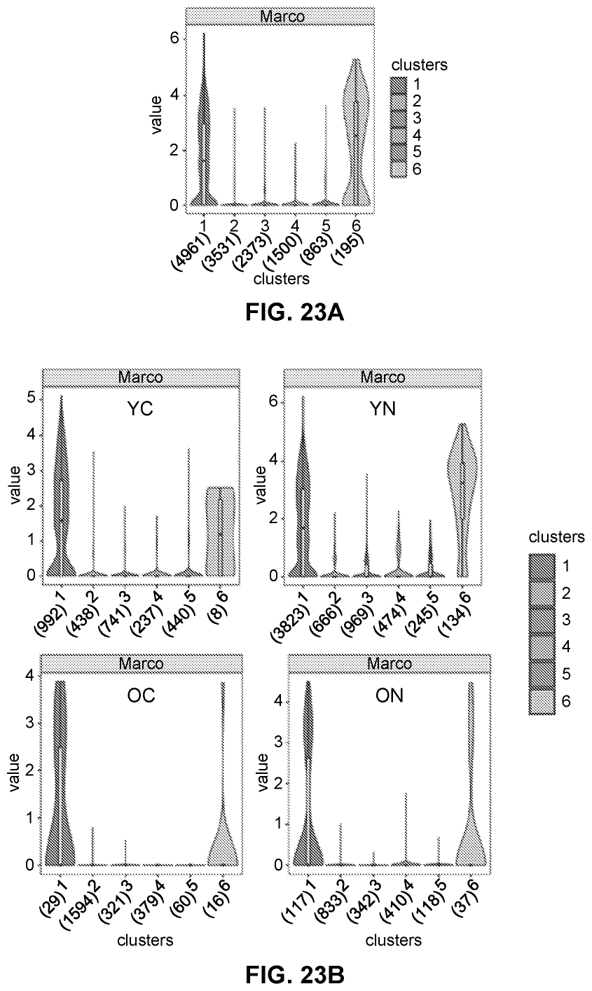

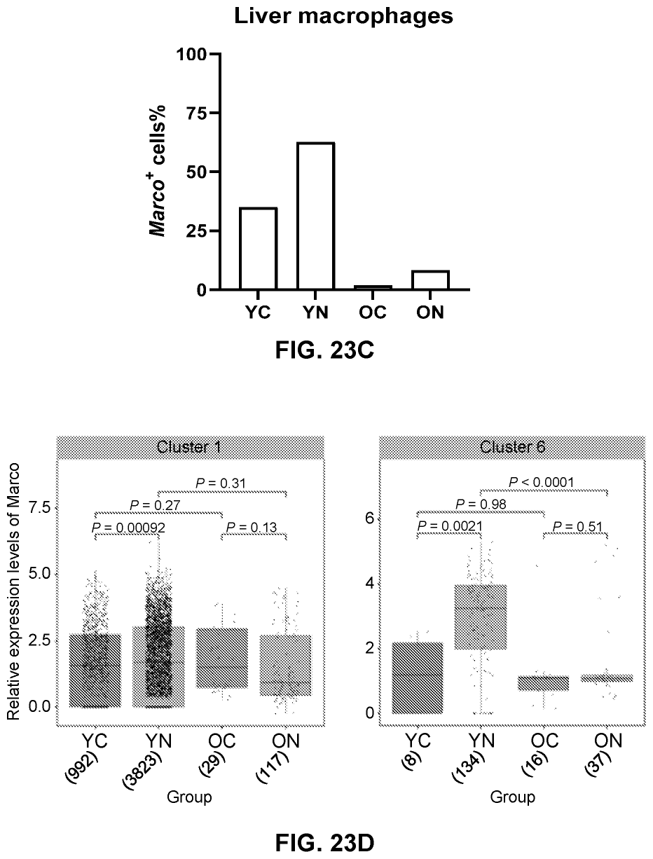

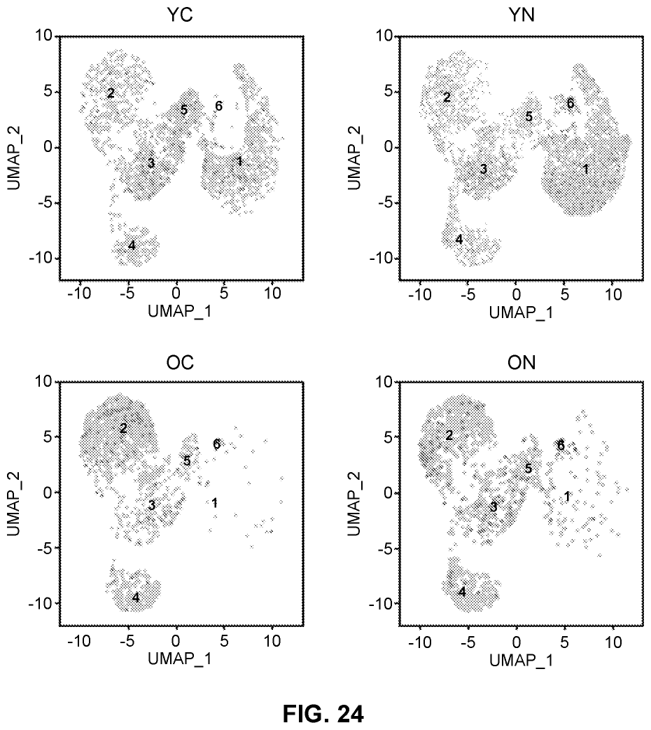

Nanoparticles induced robust increases in Marco⁺ macrophages in the young livers, but not in the old ones (FIGS. 23A-23C). Specifically, numbers of cluster 6 cells increased substantially among the young macrophages after the nanoparticle injection (FIG. 19F). This cluster was also double-positive for Marco and Mki67 (FIGS. 19F and 24), a marker of proliferating cells, suggesting that nanoparticles may stimulate the expansion of Marco macrophages. Closer analysis of expression pattern at the individual cell level in clusters 1 and 6 revealed that the Marco expression in cluster 6 of young mice with nanoparticle injection was significantly higher than that in any other groups (FIG. 23D), suggesting the capability of young livers to generate more macrophages with high Marco expression upon stimulation. Together, these single-cell transcriptional analyses revealed significant differences in how the nano-bio interface changes during aging. These findings also identified MARCO downregulation in the old livers as a potential mechanism for decreased nanoparticle uptake.

### Example 9. The Scavenger Receptor MARCO is Downregulated in Aged Macrophages Across Species

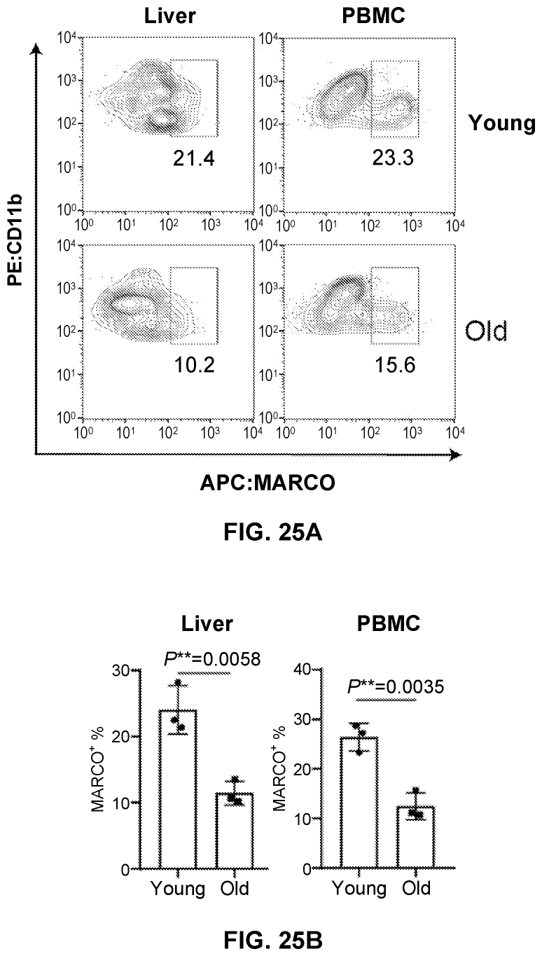

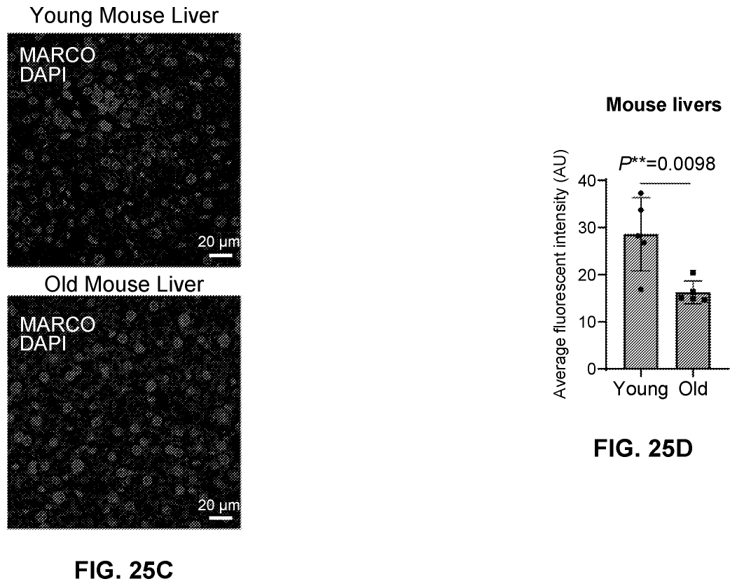

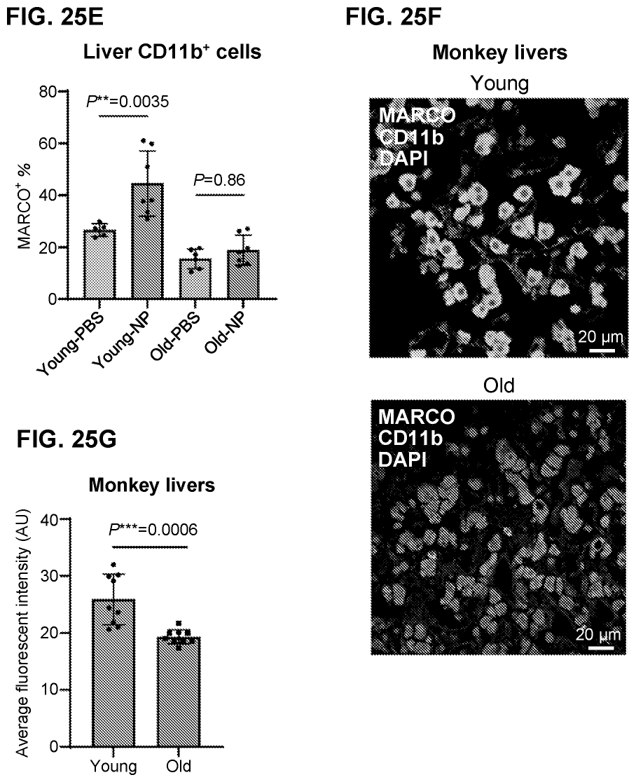

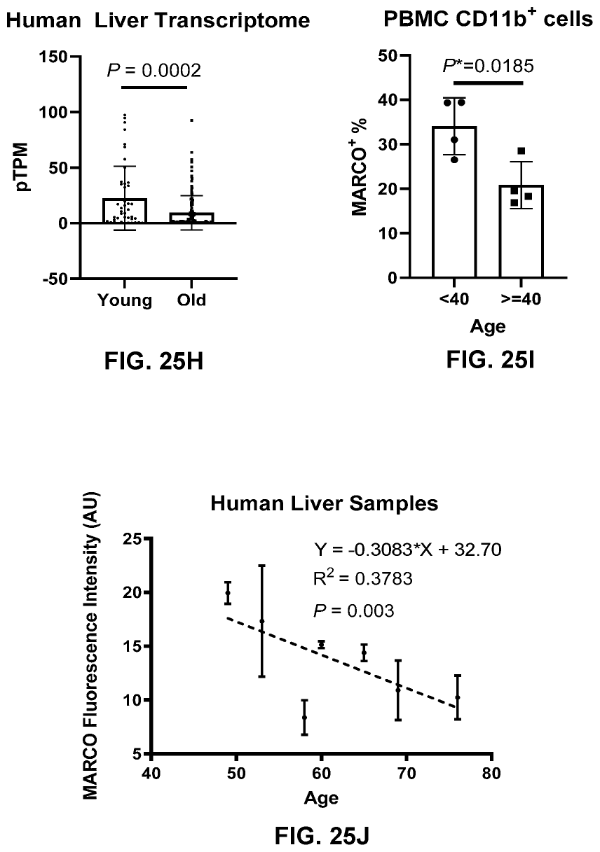

MARCO expression was evaluated in mouse tissues and it was found that MARCO expression was lower on CD11b⁺ cells from the liver and peripheral blood mononuclear cells in aged mice (FIGS. 25A-25D). Consistent with the single-cell sequencing results described above, systemic injection of nanoparticles led to significantly increased expression of MARCO on liver CD11b⁺ cells of young mice but not aged mice (FIG. 25E). Whether the downregulation of MARCO during aging is universal across different species was then evaluated. Slides of liver samples from healthy cynomolgus monkeys showed that the expression of MARCO on CD11b⁺ cells decreased with aging (FIGS. 25F-25G). The liver transcriptome data from the Human Protein Atlas showed that MARCO expression was mainly detectable in the macrophage populations (available at www.proteinatlas.org/ENSG00000019169-MARCO/single+ cell+ type/liver), and its overall liver expression was significantly lower in individuals aged>50 years (FIG. 25H).

This finding was further validated by lower MARCO expression on CD11b⁺ peripheral blood mononuclear cells (FIG. 25I) and liver tissues from older individuals (FIG. 25J). Together, these findings suggest that the aging-associated downregulation of MARCO on macrophages is conserved across several species from murine to primates.

### Example 10. MARCO Expression Correlates with Nanoparticle Uptake

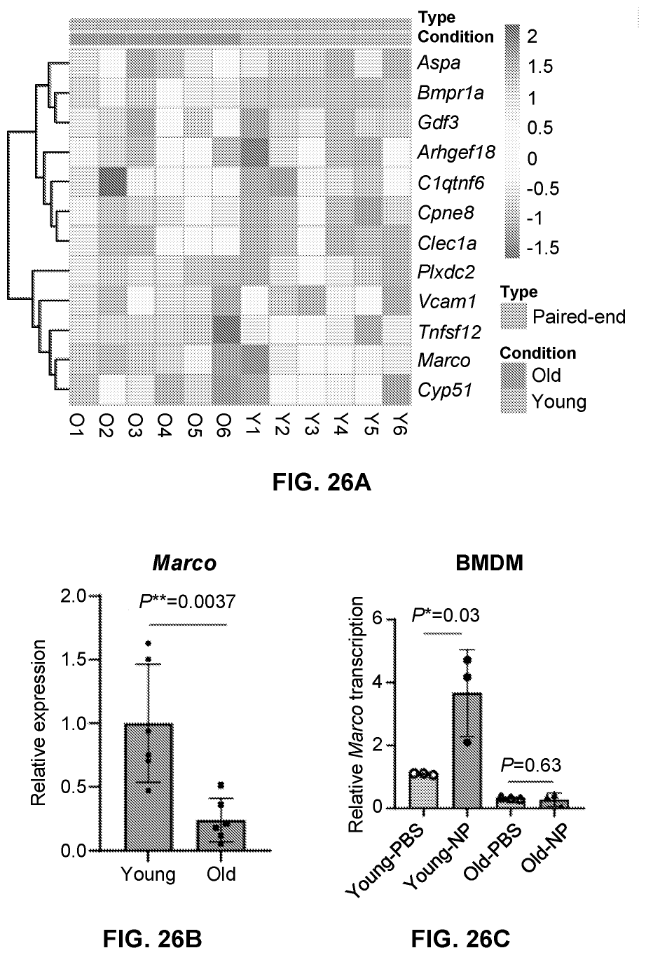

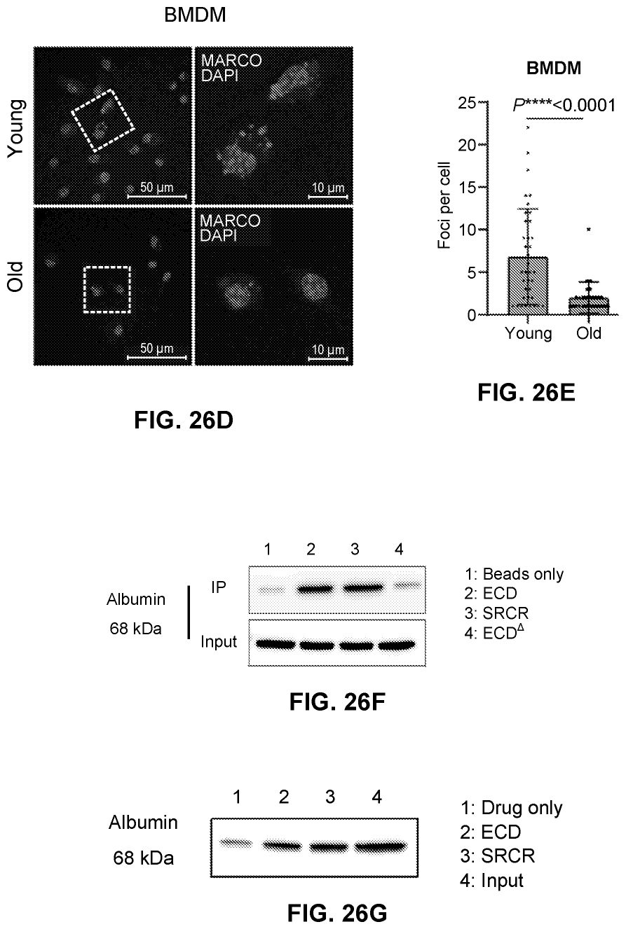

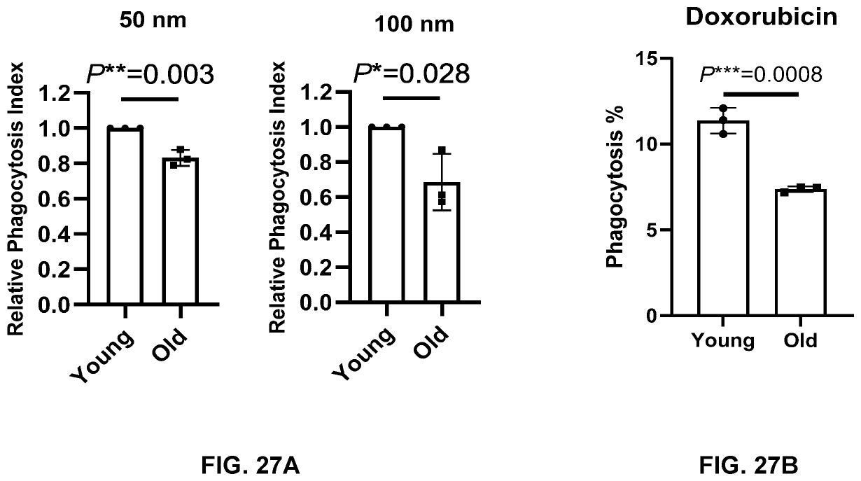

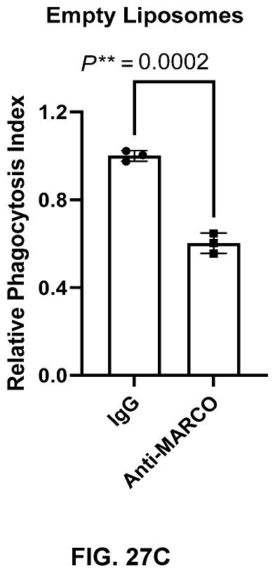

The association of MARCO with the ability of macrophages to take up nanoparticles by using bone marrow-derived macrophages (BMDMs) as a model system was further assessed. The RNAseq analysis of BMDMs described above showed that Marco expression was significantly lower in the BMDMs from the old mice (FIG. 26A-26B). These old BMDMs showed lower uptake of nanoparticles and liposomal doxorubicin (FIGS. 27A-27B). Similar to the liver macrophages, after incubation with nanoparticles in vitro, BMDMs from young mice showed increased Marco transcription, whereas the old BMDMs did not (FIG. 26C). It was also found that more MARCO foci were present in the young MARCO⁺ BMDMs than their aged counterparts (FIGS. 26D-26E), which may explain part of the difference in nanoparticle phagocytosis, because MARCO is known to cluster to form large foci to capture particles (Ojala, J. R., et al. 2007. J Biol Chem 282, 16654-16666). As described in Example 1, cells with more foci were able to capture more nanoparticles (FIG. 1A), consistent with the finding that MARCO⁺ macrophages had more robust nanoparticle uptake than did MARCO-cells (FIG. 1B).

As described in Example 2, MARCO-nanoparticle interactions can be disrupted by using an anti-MARCO antibody, reducing the uptake of nanoparticles and liposomes (FIGS. 3A-3B and 27C). Knockdown of Marco by siRNA had a similar effect (FIGS. 2A-2B). In contrast, overexpression of MARCO in aged macrophages significantly enhanced their ability to take up nanoparticles (FIGS. 1C and 26F). Together, these results confirm the role of MARCO in mediating nanoparticle uptake by macrophages, and that its downregulation may be responsible for age-associated decline in nanoparticle phagocytosis.

To better understand how MARCO interacts with nanoparticles, recombinant mouse MARCO proteins were expressed and purified. Several forms of recombinant MARCO were constructed, including the ECD (SEQ ID NO: 1), the SRCR (SEQ ID NO: 2), the ECD domain without the SRCR (ECD^{Δ}) (SEQ ID NO: 3), the SRCR domain with three arginine mutations (SRCR^{R→A}) (SEQ ID NO: 4), and the SRCR domain with the arginine-rich region deleted (SRCR^{Δ}) (SEQ ID NO: 5). The expression and purification of recombinant MARCO proteins was performed by GenScript (Piscataway, NJ, USA). The codon-optimized expression construct encoding the proteins was synthesized and cloned to expression vectors. The secretion signal peptide, along with a 6× his-tag, was added upstream of the gene. The expression plasmid was transiently expressed in Expi293 cells (Thermo Fisher Scientific) by PEI transfection. Cell culture supernatant was harvested 6 days after initial transfection. The recombinant MARCO proteins were purified by Ni-affinity chromatography with stepwise elution with 50 mM (5CV), 100 mM (5CV), and 500 mM (5CV) imidazole. The fractions were pooled together and the buffer exchange to PBS, pH 7.2 was performed by desalting columns. The purified proteins were examined by sodium dodecyl sulfate-polyacrylamide gel electrophoresis (SDS-PAGE).

Consistent with a previous report (Brannstrom, A., et al. 2002. Biochem Biophys Res Commun 290, 1462-1469), the SRCR^{R→A} and SRCR^{Δ} mutations of MARCO were not expressed. It was found that ECD and SRCR, but not ECD^{Δ}, could strongly bind with nab-paclitaxel (FIG. 26F). This suggests that the MARCO binds with ligands mainly via the SRCR domain. Next, the binding capacity of MARCO to particles and nanomedicine was tested. Centrifugal filters were used to isolate unbound MARCO after incubating the SRCR (0.1 mg/mL) with different concentrations of nanoparticles. The MARCO recombinant proteins (0.1 mg/mL SRCR, 0.5 mg/mL ECD, or 0.4 mg/mL ECD^{Δ}) were incubated with nab-paclitaxel (5 mg/mL, paclitaxel concentration). After 15 min at room temperature with gentle shaking, the mixture was further incubated with the Dynabeads His-Tag isolation and Pulldown (ThermoFisher, Cat. #10103D). After magnetic separation and three washes with PBS, the beads were boiled in western blot sample buffer for subsequent western blotting. Primary antibodies used for western blots were albumin, CST, Cat. #4929, 1:1000; and His-Tag, CST, Cat. #2365, 1:1000. To test the binding capacity of MARCO, 0.1 mg/mL of SRCR was incubated for 15 min at room temperature with different concentrations of nab-paclitaxel or nanoparticles. The mixture was centrifuged through a 30-kDa cutoff filter (Millipore, UFC903096) at 1000 g for 30 minutes. The unbound SRCR in the lower part of the tube was examined by western blotting. To test the binding of nanoparticles with additional MARCO after saturation, the ECD was coated onto assay plates (Corning, CLS3590) for 1 h at room temperature, washed with TBST (CST, 9997) and blocked with Blocking Buffer (ThermoFisher, 37535) for 1 h at room temperature. Nab-paclitaxel (20 μg/mL) was pre-incubated with or without saturation doses of SRCR (0.4 μg/mL) or ECD (2 μg/mL) for 15 min at room temperature before being added to the plates. After incubation at room temperature for 1 hour with shaking, the supernatant was aspirated and examined by western blotting.

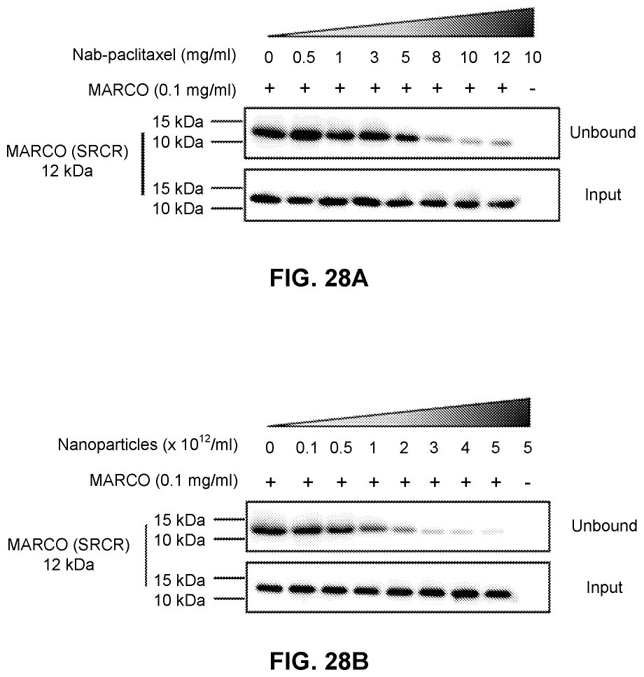

Concentrations of nab-paclitaxel exceeding 8 mg/mL or nanoparticles exceeding 3×10¹² particles/mL could not further reduce the level of unbound MARCO detected (FIGS. 28A-28B), implying that these concentrations were close to saturation. Further tests showed that nanoparticles could not bind with additional MARCO after being saturated with recombinant MARCO (FIG. 3C and FIG. 26G). Together, these results indicate that MARCO is mainly interacting with nanoparticles via the SRCR domain, and that disruption of MARCO binding can reduce the uptake of nanoparticles by macrophages.

### Example 11. MARCO Blockade Improves Nanomedicine Efficacy and Reduces Drug Toxicity in Young Mice

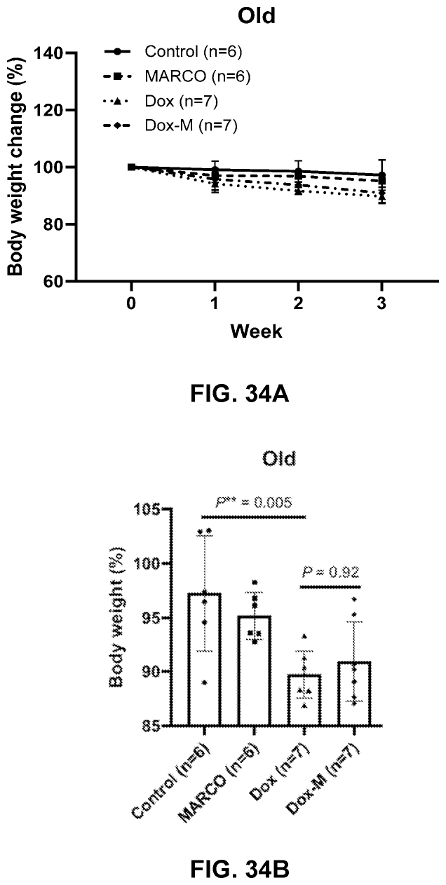

To evaluate whether MARCO blockade could improve the effectiveness of other nanotherapeutics, mice were treated with liposomal doxorubicin, as described above in Examples 5 and 6. MARCO blockade led to improved antitumor efficacy and reduced toxicity in young mice, but not in old ones (FIGS. 9A-9D, 33A, and 34A-34B).

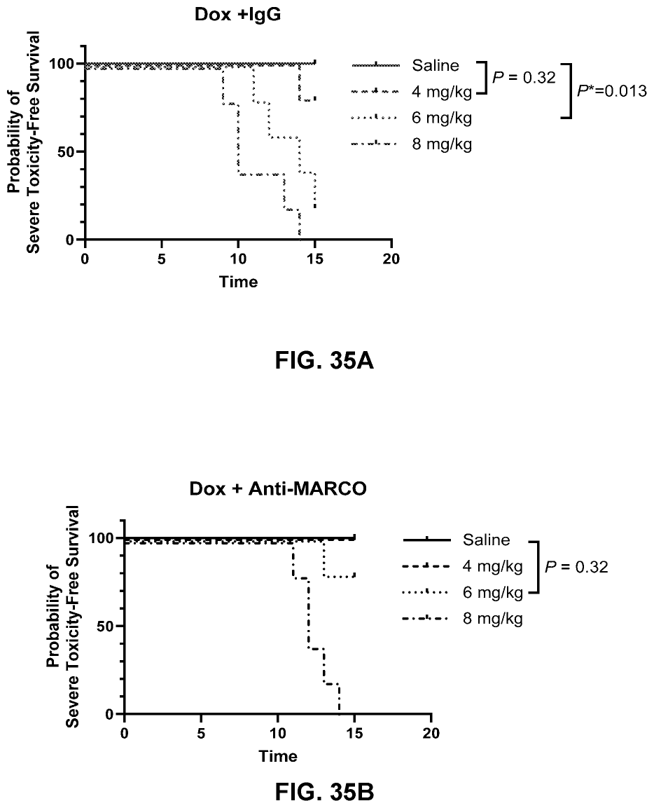

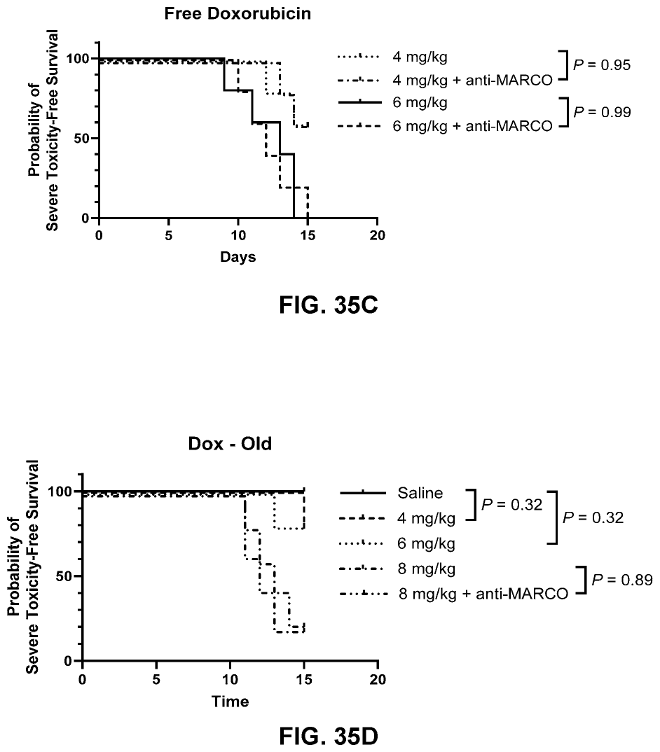

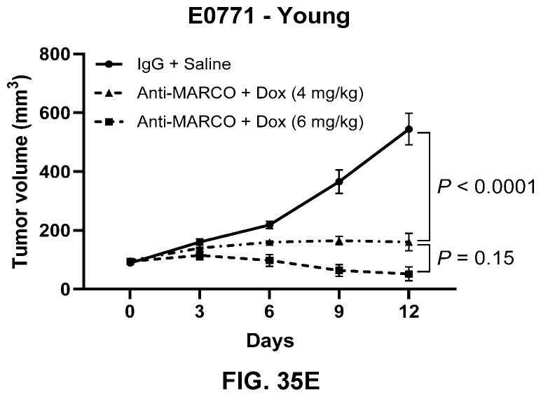

Based on the observation that MARCO blockade reduced the toxicity, it was assessed if the mice could tolerate higher dose of nanotherapeutics. Anti-MARCO antibody increased the liposomal doxorubicin, but not free doxorubicin, tolerance of young mice (FIGS. 35A-35C). The old mice could tolerate higher dose than young mice, but MARCO blockade was unable to further increase the tolerance (FIG. 35D). When the young mice were treated with a higher dose of liposomal doxorubicin in combination of anti-MARCO, tumor regression was achieved (FIG. 35E). These results support that blocking MARCO in young hosts reduces the liver uptake and clearance, enhances the intratumoral delivery, improves the effectiveness, and reduces the toxicity of nanotherapeutics.

What is claimed is:

1. A method of treating a tumor or cancer in a subject in need thereof comprising administering to the subject a therapeutically effective amount of a combination therapy comprising a first agent and a second agent, wherein the first agent is capable of blocking the interaction between a Macrophage Receptor with Collagenous Structure (MARCO) and a nanoparticle, and the second agent comprises an anti-cancer agent or an anti-tumor agent, and wherein at least the second agent is in the form of a nanoformulation.

2. A method for reducing toxicity to a nanoformulated therapy in a subject in need thereof comprising administering to the subject a therapeutically effective amount of a combination therapy comprising a first agent and a second agent, wherein the first agent is capable of blocking the interaction between a Macrophage Receptor with Collagenous Structure (MARCO) and a nanoparticle and the second agent comprises an anti-cancer agent or an anti-tumor agent, wherein at least the second agent is in the form of a nanoformulation.

3. The method of claim 1 or 2, wherein method allows for an increase in the dosage or frequency in dosage of the second agent without an increase in toxicity to the subject relative to administering the nanoformulated second agent without the first agent.

4. The method of any one of claims 1-3, wherein the first agent decreases MARCO activity according to one or more of: (i) decreasing the amount of transcription of a MARCO encoding gene, (ii) decreasing the amount of an mRNA or a pre-mRNA encoding a MARCO protein, (iii) decreasing the amount of a MARCO protein, (iv) decreasing the amount of MARCO protein localized to a cell surface, and (v) decreasing the fraction of MARCO which is not bound to a ligand or other binding partner.

5. The method of any one of claims 1-4, wherein the nanoformulation comprises a nanoparticle or an exosome.

6. The method of any one of claims 1-4, wherein the nanoformulation comprises a nanoparticle is selected from the group consisting of an organic nanoparticle (optionally, a liposome-based nanoparticles, a polymer-based nanoparticles, a dendrimers, or any combination thereof), an inorganic nanoparticle, a hybrid nanoparticle, and any combinations thereof.

7. The method of claim 6, wherein the nanoformulation comprises a lipid nanoparticle.

8. The method of claim 6, wherein the nanoformulation comprises a polymer-based nanoparticle (optionally, a polymeric nanoparticle, a polymeric micelle, or any combination thereof).

9. The method of any one of claims 1-8, wherein prior to administration the subject expresses MARCO at or above a reference MARCO expression level.

10. The method of any one of claims 1-9, wherein the subject is less than 50 years, less than 45 years, less than 40 years, less than 35 years, or less than 30 years in age.

11. The method of any one of claims 1-10, wherein the subject is between 1-50 years old.

12. The method of any one of claims 1-11, wherein the subject is a pediatric patient, an adolescent patient, or a young adult patient.

13. The method of any one of claims 1-12, wherein the first agent comprises a MARCO inhibitor or MARCO blocking agent.

14. The method of any one of claims 1-13, wherein the first agent is a MARCO blocking agent comprising a recombinant MARCO protein or a portion thereof.

15. The method of claim 14, wherein the recombinant MARCO protein or the portion thereof comprises: an extracellular domain (ECD) of MARCO, or a scavenger receptor cysteine-rich (SRCR) domain.

16. The method of any one of claims 1-13, wherein the first agent comprises an antibody.

17. The method of claim 16, wherein the antibody is a monoclonal antibody or a polyclonal antibody.

18. The method of any one of claims 1-13, wherein the first agent comprises an mRNA silencing polynucleotide.

19. The method of claim 18, wherein the mRNA silencing polynucleotide is a siRNA, a shRNA, or a miRNA.

20. The method of claim 19, wherein the mRNA silencing polynucleotide is a siRNA.

21. The method of any one of claims 1-20, wherein the MARCO activity in the subject after administration of the first agent is at least 1%, 5%, 10%, 15%, 20%, 25%, 30%, 40%, 50%, 60%, 70%, 80%, 90%, 95%, 99%, 99.5%, or 99.9% less than the MARCO activity prior to administration of the first agent.

22. The method of any one claims 1-21, wherein the cancer is selected from the group consisting of brain cancer, breast cancer, cervical cancer, colorectal cancer, esophageal cancer, lung cancer, non-small cell lung cancer, lymphoma, ovarian cancer, pancreatic cancer, prostate cancer, stomach cancer, thyroid cancer, bladder cancer, and uterine cancer.

23. The method of any one of claims 1-22, wherein the cancer is breast cancer.

24. The method of any one of claims 1-22, wherein the cancer is lung cancer.

25. The method of any one of claims 1-24 wherein the cancer is a metastatic cancer.

26. The method of any one of claims 1-25, wherein the first agent and the nanoformulated second agent are administered together in a single composition.

27. The method of any one of claims 1-25, wherein the first agent and the nanoformulated second agent are administered in separate compositions.

28. The method of any one of claims 1-27, wherein the second agent comprises a therapeutically effective dosage which is lower than a therapeutically effective dosage of the nanoformulated second agent administered as a monotherapy without the first agent.

29. The method of any one of claims 1-28, wherein the nanoformulated second agent is less toxic to the subject when administered as the combination therapy than as a monotherapy without the first agent.

30. The method of any one of claims 1-29, wherein the nanoformulated second agent when administered as the combination therapy is less toxic to the subject's liver and/or kidney than the same dosage of the second agent administered as a monotherapy without the first agent.

31. The method of any one of claims 1-30, further comprising detecting the level MARCO in a sample from the subject prior to administration of the combination therapy.

32. The method of claim 31, wherein the combination therapy is administered when the MARCO level is high in the subject.

33. The method of claim 31, wherein the sample is a blood sample or a urine sample.

34. The method of any one of claims 1-32, wherein the combination therapy is administered systemically to the subject.

35. The method of any one of claims 1-34, wherein the combination therapy is administered to the subject by intravenous, subcutaneous, or intramuscular injection.

36. The method of any one of claims 1-35, wherein the subject has a low tolerance to the nanoformulated second agent when administered as a monotherapy without the first agent.

37. The method of any one of claims 1-36, wherein the second agent is selected from the group consisting of 5-fluorouracil, afatinib, aplidin, azaribine, anastrozole, anthracyclines, axitinib, AVL-101, AVL-291, bendamustine, bleomycin, bortezomib, bosutinib, bryostatin-1, busulfan, calicheamycin, camptothecin, carboplatin, 10-hydroxycamptothecin, carmustine, celecoxib, chlorambucil, cisplatinum, COX-2 inhibitors, irinotecan (CPT-11), SN-38, carboplatin, cladribine, camptothecans, crizotinib, cyclophosphamide, cytarabine, dacarbazine, dasatinib, dinaciclib, docetaxel, dactinomycin, daunorubicin, DM1, DM3, DM4, doxorubicin, 2-pyrrolinodoxorubicine (2-PDox), a pro-drug form of 2-PDox (pro-2-PDox), cyano-morpholino doxorubicin, doxorubicin glucuronide, endostatin, epirubicin glucuronide, erlotinib, estramustine, epidophyllotoxin, erlotinib, entinostat, estrogen receptor binding agents, etoposide (VP16), etoposide glucuronide, etoposide phosphate, exemestane, fingolimod, floxuridine (FUdR), 3′,5′-O-dioleoyl-FudR (FUdR-dO), fludarabine, flutamide, farnesyl-protein transferase inhibitors, flavopiridol, fostamatinib, ganetespib, GDC-0834, GS-1101, gefitinib, gemcitabine, hydroxyurea, ibrutinib, idarubicin, idelalisib, ifosfamide, imatinib, lapatinib, lenolidamide, leucovorin, LFM-A13, lomustine, mechlorethamine, melphalan, mercaptopurine, 6-mercaptopurine, methotrexate, mitoxantrone, mithramycin, mitomycin, mitotane, monomethylauristatin F (MMAF), monomethylauristatin D (MMAD), monomethylauristatin E (MMAE), navelbine, neratinib, nilotinib, nitrosurea, olaparib, plicomycin, procarbazine, paclitaxel, PCI-32765, pentostatin, PSI-341, raloxifene, semustine, SN-38, sorafenib, streptozocin, SU11248, sunitinib, tamoxifen, temazolomide, transplatinum, thalidomide, thioguanine, thiotepa, teniposide, topotecan, uracil mustard, vatalanib, vinorelbine, vinblastine, vincristine, vinca alkaloids, ZD1839, and any combination thereof.

38. The method of any one of claims 1-37, wherein the second agent comprises paclitaxel.

39. The method of any one of claims 1-37, wherein the second agent comprises doxorubicin.

40. The method of any one of claims 1-37, wherein the second agent comprises irinotecan.

41. The method of any one of claims 1-37, wherein the second agent comprises albumin-bound paclitaxel.

42. The method of any one of claims 1-37, wherein the second agent comprises PEGylated liposomal doxorubicin.

43. The method of any one of claims 1-37, wherein the second agent comprises liposomal irinotecan.

44. A combination therapy comprising a first agent and a second agent, wherein the first agent is capable of blocking the interaction between a Macrophage Receptor with Collagenous Structure (MARCO) and a nanoparticle, and the second agent comprises an anti-cancer agent or an anti-tumor agent, and wherein at least the second agent is in the form of a nanoformulation.

45. The combination therapy of claim 44, wherein the first agent is capable of decreasing MARCO activity according to one or more of: (i) decreasing the amount of transcription of a MARCO encoding gene, (ii) decreasing the amount of an mRNA or a pre-mRNA encoding a MARCO protein, (iii) decreasing the amount of a MARCO protein, (iv) decreasing the amount of MARCO protein localized to a cell surface, and (v) decreasing the fraction of MARCO which is not bound to a ligand or other binding partner.

46. The combination therapy of claim 44 or 45, wherein the nanoformulation comprises an exosome.

47. The combination therapy of claim 44 or 45, wherein the nanoformulation comprises a nanoparticle.

48. The combination therapy of claim 47, wherein the nanoformulation comprises a lipid nanoparticle.

49. The combination therapy of claim 47, wherein the nanoformulation comprises a protein nanoparticle.

50. The combination therapy of any one of claims 44-49, wherein the first agent comprises a MARCO inhibitor or MARCO blocking agent.

51. The combination therapy of any one of claims 44-50, wherein the first agent is a MARCO blocking agent comprising a recombinant MARCO protein or a portion thereof.

52. The combination therapy of claim 51, wherein the recombinant MARCO protein or the portion thereof comprises: an extracellular domain (ECD) of MARCO, or a scavenger receptor cysteine-rich (SRCR) domain.

53. The combination therapy of any one of claims 44-50, wherein the first agent comprises an antibody.

54. The combination therapy of claim 53, wherein the antibody is a monoclonal antibody or a polyclonal antibody.

55. The combination therapy of any one of claims 44-50, wherein the first agent comprises an mRNA silencing polynucleotide.

56. The combination therapy of claim 55, wherein the mRNA silencing polynucleotide is a siRNA, a shRNA, or a miRNA.

57. The combination therapy of claim 55 or 56, wherein the mRNA silencing polynucleotide is a siRNA.

58. The combination therapy of any one of claims 44-57, wherein the first agent and the nanoformulated second agent are formulated together in a single composition.

59. The combination therapy of any one of claims 44-57, wherein the first agent and the nanoformulated second agent are formulated as separate compositions.

60. The combination therapy of any one of claims 44-59, wherein the second agent comprises a therapeutically effective dosage which is lower than a therapeutically effective dosage of the nanoformulated second agent administered as a monotherapy without the first agent.

61. The combination therapy of any one of claims 44-60, wherein the nanoformulated second agent is less toxic to the subject when administered as the combination therapy than as a monotherapy without the first agent.

62. The combination therapy of any one of claims 44-61, wherein the combination therapy is formulated for intravenous, subcutaneous, or intramuscular injection.

63. The combination therapy of any one of claims 44-62, wherein the second agent is selected from the group consisting of 5-fluorouracil, afatinib, aplidin, azaribine, anastrozole, anthracyclines, axitinib, AVL-101, AVL-291, bendamustine, bleomycin, bortezomib, bosutinib, bryostatin-1, busulfan, calicheamycin, camptothecin, carboplatin, 10-hydroxycamptothecin, carmustine, celecoxib, chlorambucil, cisplatinum, COX-2 inhibitors, irinotecan (CPT-11), SN-38, carboplatin, cladribine, camptothecans, crizotinib, cyclophosphamide, cytarabine, dacarbazine, dasatinib, dinaciclib, docetaxel, dactinomycin, daunorubicin, DM1, DM3, DM4, doxorubicin, 2-pyrrolinodoxorubicine (2-PDox), a pro-drug form of 2-PDox (pro-2-PDox), cyano-morpholino doxorubicin, doxorubicin glucuronide, endostatin, epirubicin glucuronide, erlotinib, estramustine, epidophyllotoxin, erlotinib, entinostat, estrogen receptor binding agents, etoposide (VP16), etoposide glucuronide, etoposide phosphate, exemestane, fingolimod, floxuridine (FUdR), 3′,5′-O-dioleoyl-FudR (FUdR-dO), fludarabine, flutamide, farnesyl-protein transferase inhibitors, flavopiridol, fostamatinib, ganetespib, GDC-0834, GS-1101, gefitinib, gemcitabine, hydroxyurea, ibrutinib, idarubicin, idelalisib, ifosfamide, imatinib, lapatinib, lenolidamide, leucovorin, LFM-A13, lomustine, mechlorethamine, melphalan, mercaptopurine, 6-mercaptopurine, methotrexate, mitoxantrone, mithramycin, mitomycin, mitotane, monomethylauristatin F (MMAF), monomethylauristatin D (MMAD), monomethylauristatin E (MMAE), navelbine, neratinib, nilotinib, nitrosurea, olaparib, plicomycin, procarbazine, paclitaxel, PCI-32765, pentostatin, PSI-341, raloxifene, semustine, SN-38, sorafenib, streptozocin, SU11248, sunitinib, tamoxifen, temazolomide, transplatinum, thalidomide, thioguanine, thiotepa, teniposide, topotecan, uracil mustard, vatalanib, vinorelbine, vinblastine, vincristine, vinca alkaloids, ZD1839, and any combination thereof.

64. The combination therapy of any one of claims 44-63, wherein the second agent comprises paclitaxel.

65. The combination therapy of any one of claims 44-63, wherein the second agent comprises doxorubicin.

66. The combination therapy of any one of claims 44-63, wherein the second agent comprises irinotecan.

67. The combination therapy of any one of claims 44-63, wherein the second agent comprises albumin-bound paclitaxel.

68. The combination therapy of any one of claims 44-63, wherein the second agent comprises PEGylated liposomal doxorubicin.

69. The combination therapy of any one of claims 44-63, wherein the second agent comprises liposomal irinotecan.

70. A kit comprising a first agent and a nanoformulated second agent, wherein the first agent and the nanoformulated second agent can be combined to produce a combination therapy, wherein the first agent is capable of blocking the interaction between a Macrophage Receptor with Collagenous Structure (MARCO) and a nanoparticle, and the nanoformulated second agent comprises an anti-cancer agent or an anti-tumor agent.

71. The kit of claim 70, wherein the combination therapy is the combination therapy of any one of claims 44-69.

72. The method of any one of claims 1-43, or the combination therapy of any one of claims 44-69, or the kit of any one of claims 70-71, wherein the nanoformulated second agent is capable of being metabolized in the liver of a/the subject.

73. The method of any one of claims 1-43, or the combination therapy of any one of claims 44-69, or the kit of any one of claims 70-71, wherein the second agent is a chemotherapeutic agent.

74. The method of any one of claims 1-43, or the combination therapy of any one of claims 44-69, or the kit of any one of claims 70-71, wherein the second agent is a polynucleotide.

75. The method of any one of claims 1-43, or the combination therapy of any one of claims 44-69, or the kit of any one of claims 70-71, wherein the second agent is a polynucleotide, and wherein the polynucleotide is an RNA molecule or a DNA molecule.

76. The method of any one of claims 1-43, or the combination therapy of any one of claims 44-69, or the kit of any one of claims 70-71, wherein the second agent comprises paclitaxel, doxorubicin, irinotecan, albumin-bound paclitaxel, PEGylated liposomal doxorubicin, liposomal irinotecan, or any combination thereof.

77. The method, or the combination therapy, or the kit of any one of claims 1-76, wherein the first agent is a/the MARCO inhibitor or MARCO blocking agent.

78. The method, or the combination therapy, or the kit of any one of claims 1-77, wherein the first agent is selected from the group consisting of a/the recombinant MARCO protein or a portion thereof comprising an extracellular domain (ECD) of MARCO and/or a scavenger receptor cysteine-rich (SRCR) domain; an/the antibody; and a/the mRNA silencing polynucleotide.

79. The method of any one of claims 1-43, or the combination therapy of any one of claims 44-69, wherein the first agent is capable of increasing the blood circulation time of the second agent.
